(19) United States
(12) Patent Application Publication
(10) Pub. No.: US 2025/0268863 A1
(43) Pub. Date: Aug. 28, 2025

(54) DOSING REGIMENS FOR TREATMENT OF PATIENTS WITH LOCALLY ADVANCED SQUAMOUS CELL CARCINOMA

(71) Applicant: DEBIOPHARM INTERNATIONAL SA, Lausanne (CH)

(72) Inventors: Silvano BRIENZA, Paris (FR); Claudio ZANNA, Lausanne (CH); Sergio Adrian SZYLDERGEMAJN ALTMAN, Saint-Prex (CH); Jean BOURHIS, Lausanne (CH)

(21) Appl. No.: 17/761,503

(22) PCT Filed: Sep. 25, 2020
(86) PCT No.: PCT/EP2020/076994

§ 371 Date: Mar. 17, 2022

Related U.S. Application Data
(60) Provisional application No. 63/016,762, filed on Apr. 28, 2020.
(60) Provisional application No. 62/905,703, filed on Sep. 25, 2019.

(30) Foreign Application Priority Data
Jul. 7, 2020 (EP) 20184601.1

(51) Int. Cl. A61K 31/407; A61K 31/282; A61K 33/243; A61N 5/10; A61P 35/00

(52) U.S. Cl. CPC A61K 31/407; A61K 31/282; A61K 33/243; A61P 35/00; A61N 5/10

(57) ABSTRACT

Methods of administering inhibitors of apoptosis protein (“IAP”), or pharmaceutically acceptable salts thereof, for the treatment of a human patient having locally advanced squamous cell carcinoma are provided.

### FIELD OF THE INVENTION

The field of the invention generally relates to methods of treating a human patient having locally advanced squamous cell carcinoma comprising administering to the human patient, in need thereof, one or more inhibitors of apoptosis protein (“IAP”) antagonists, such as (5S,8S,10aR)—N-benzhydryl-5-[[(2S)-2-(methylamino)propanoyl]amino]-3-(3-methylbutanoyl)-6-oxo-1,2,4,5,8,9,10,10a-octahydropyrrolo[1,2-a][1,5]diazocine-8-carboxamide or {(5S,8S,10aR)—N-benzhydryl-5-((S)-2-(methylamino)propanamido)-3-(3-methylbutanoyl)-6-oxodecahydropyrrolo[1,2-a][1,5]diazocine-8-carboxamide} (CAS No. 1071992-99-8, also known as Debio 1143 or xevinapant, hereinafter referred to as Compound A), or pharmaceutically acceptable salt thereof, having the structure below

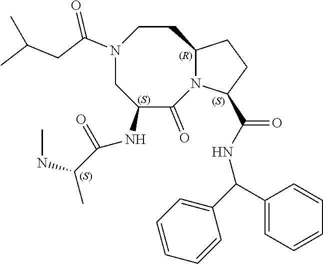

LCL-161 (CAS No. 1005342-46-0), or pharmaceutically acceptable salt thereof, CUDC 427/GDC 0917 (CAS No. 1446182-94-0), or pharmaceutically acceptable salt thereof, birinapant (CAS No. 1260251-31-7, also known as TL-32711), or pharmaceutically acceptable salt thereof, AZD5582 (CAS No. 1258392-53-8), or pharmaceutically acceptable salt thereof, APG-1387 (CAS No. 1570231-89-8), or pharmaceutically acceptable salt thereof, ASTX660 (CAS No. 1799328-86-1), or pharmaceutically acceptable salt thereof, SBP-0636457 (CAS No. 1422180-49-1), and/or JP1201 (Joyant Pharmaceuticals), or pharmaceutically acceptable salt thereof.

### BACKGROUND OF THE INVENTION

Squamous cell carcinoma (“SCCHN”) is the sixth most common cancer worldwide and more than half of the locally advanced patients will have a relapse or failure within five years of treatment. Head and neck cancer include a variety of epithelial tumors originating in the lip, oral cavity, hypopharynx, oropharynx, nasopharynx or larynx. Most head and neck cancers (90% to 95%) are SCCHN. Primary tumor sites may include oral cavity, oropharynx, hypopharynx, or larynx, but most patients are diagnosed with oropharyngeal cancers (OPC).

Approximately two-third of those patients are diagnosed with locally advanced disease (LA-SCCHN), which is associated to poor survival outcomes with an average survival of 5-year. Clinical symptoms are varied and include gradually progressing impairment in respiration, chewing, swallowing, tasting, smelling, speaking and hearing. Head and neck cancers result in destructive disease, tend to develop local lymph node metastases early and can develop distant metastases at relatively late stages even after effective local therapy. LA-SCCHN treatments, such as surgery or chemotherapy, often lead to difficulties in chewing, swallowing, and breathing, thereby affecting fundamental features of human existence. No major innovations have entered the field in LA-SCCHN treatment for years outside of new surgical methods. However, many LA-SCCHN patients are ineligible for tumor resection. In addition, surgery is associated with severe negative impact on patients' lives. A patient's sense of taste, smell and hearing can be severely compromised along with uniquely human characteristics such as appearance and voice, leading to impairment of social functioning and severe psychological issues. Severe functional impairments affect particularly human papilloma (“HPV”)-negative oropharyngeal (“OPC”) cancer and non-OPC SCCHN. Non-surgical treatments, especially platinum-based chemoradiotherapy (“CRT”), are extremely difficult for patients to endure, due to extensive negative side effects. Reviews on state of the art knowledge on LA-SCCHN and its treatments are provided in T. Y. Seiwert and E. E. W. Cohen “State-of-the-art management of locally advanced head and neck cancer” in British Journal of Cancer (2005) 92, 1341-1348 and M. Mañós et al. “Multidisciplinary management of head and neck cancer: First expert consensus using Delphi methodology from the Spanish Society for Head and Neck Cancer (part 1)” in Oral Oncology, Volume 70, July 2017, Pages 58-64.

OPC patients can be classified as low, intermediate and high-risk patients, according to the presence of prognostic factors such as HPV status and alcohol or tobacco abuse. Patients with HPV negative OPC are considered to have a worse prognosis, with HPV status being considered to be a strong and independent prognostic factor for survival. Those patients require more and better tailored options, to improve overall treatment outcomes.

Viral etiology, namely human papilloma virus (HPV) infection, predominantly HPV type 16 (p16), and Epstein-Barr virus (EBV) are linked to the carcinogenesis/transformation process. However, patients with HPV negative OPC are considered to have a worse prognosis; in fact, negative HPV tumors in patients with stage III or IV oropharyngeal SCCHN was shown to be associated with a worse overall survival rate (57.1% vs 82.4% in HPV positive tumors) and a worse 3-year rate of locoregional disease without distant metastasis (35.1% vs 13.6%) (Ang et al. N Engl J Med. 2010 Jul. 1; 363(1): 24-35. doi:10.1056/NEJMoa0912217).

HPV-negative alcohol and/or tobacco-related SCCHN frequently harbors mutations of the p53 gene and downregulation of the p16 protein, linked to poor prognosis due to early CRT resistance and treatment failures as well as limited survival. In fact, patients with a smoking history of more than 10 pack-years and HPV-negative OPC have a median OS slightly over 24 months and a risk of death and cancer relapse increased by 1% for each additional pack-year of tobacco smoking. The term “pack-year” is well-known and understood by the person skilled in the art, for example as defined in Table 1 of R. Wender et al. “American Cancer Society Lung Cancer Screening Guidelines” in CA Cancer J Clin. 2013 March-April; 63(2): 107-117 and in the background section of L. M. Fucito et al “Pairing Smoking-Cessation Services With Lung Cancer Screening: A Clinical Guideline From the Association for the Treatment of Tobacco Use and Dependence and the Society for Research on Nicotine and Tobacco” in Cancer. 2016 Apr. 15; 122(8): 1150-1159, as 1 pack-year corresponds to smoking 1 pack per day for 1 year.

Therefore, improvement in SCCHN therapy to enhance treatment outcomes in patients with unresectable, locally advanced, HPV negative status tumor, and without generating incremental detriment to patients' quality of life, is a priority.

Therefore, there remains a need for treatment of patients with local advanced squamous cell carcinoma, particularly those that are high risk—including heavy smokers, heavy consumers of alcohol, and/or patients with HPV-negative OPC.

### BRIEF SUMMARY OF THE DISCLOSURE

The present invention provides means and methods for the treatment of locally advanced squamous cell carcinoma. In particular, multiple aspects of two main embodiments of the invention are disclosed. These two main embodiments are identified hereinbelow as embodiments A and B.

Embodiment A relates to the following specific aspects.

Methods for treating human patients having locally advanced squamous cell carcinoma comprising administering to the patient, in need thereof, Compound A, or pharmaceutically acceptable salt thereof, in an amount corresponding to about 100 mg to about 500 mg of Compound A as its free base, daily, wherein the patient in need thereof has a positive smoking history and/or is a heavy consumer of alcohol are provided herein. In some aspects, the human patient has not previously received treatment (e.g., surgery, chemotherapy, radiotherapy, immunotherapy) of the locally advanced squamous cell carcinoma.

In some aspects, the human patient is administered about 200 mg of Compound A, or pharmaceutically acceptable salt, based on its free base.

In some aspects, the human patient has a positive smoking history. In some aspects, the human patient is a heavy consumer of alcohol.

In some aspects, Compound A is in free base form. In some aspects, Compound A is a pharmaceutically acceptable salt. In some aspects, Compound A is a pharmaceutically acceptable salt form selected from the group consisting of acetate, hydrochloride, citrate, lactate, fumarate, succinate, phosphate, maleate, sulfate, tartrate, benzoate, mesylate, malate, hydrobromide, tosylate, nitrate, N-acetylglycine, ascorbate, butanate, ethane-1,2-disulfonate, gentisate, glucoronate, glutarate, glycolate, isethionate, ketoglutarate, malonate, napadisylate, napsylate, nicotinate, pyroglutamate, pyruvate, sebacate, and succinate. In some aspects, the pharmaceutically acceptable form is selected from the group consisting of sulfate, tosylate, gentisate, hydrochloride, napadisylate, napsylate, laurylsulfate, xinafoate, and pamoate.

In some aspects, Compound A, or pharmaceutically acceptable salt thereof, is administered orally. In some aspects, Compound A, or pharmaceutically acceptable salt thereof, is administered as a solid dosage form. In some aspects, the solid dosage form is a capsule or tablet.

In some aspects, Compound A, or pharmaceutically acceptable salt thereof, is administered as a solution. In some aspects, the solution is an aqueous solution. In some aspects, Compound A, or pharmaceutically acceptable salt thereof, is administered orally via a nasogastric tube, percutaneous endoscopic gastrostomy tube, or percutaneous endoscopic jejunostomy tube. In some aspects, the solution (e.g., aqueous solution) comprises Compound A, or pharmaceutically acceptable salt thereof, at a concentration of between about 10 mg/mL to about 50 mg/mL based on Compound A as its free base. In some aspects, the solution (e.g., aqueous solution) comprises Compound A, or pharmaceutically acceptable salt thereof, at a concentration of about 20 mg/mL based on Compound A, as its free base.

In some aspects, Compound A, or pharmaceutically acceptable salt thereof, is administered as one dose one time per day. In some aspects, Compound A, or pharmaceutically acceptable salt thereof, is divided into multiple doses that are administered one, two, three, or four times per day.

In some aspects, the human patient is also administered a platinum-based chemotherapeutic drug. In some aspects, the platinum-based chemotherapeutic drug is intravenously administered. In some aspects, the platinum-based chemotherapeutic drug is one or more selected from the group consisting of cisplatin, carboplatin, oxaliplatin, and nedaplatin.

In some aspects, the platinum-based chemotherapeutic drug is cisplatin. In some aspects, the cisplatin is administered at a dose between about 10 mg/m² weekly and 150 mg/m² weekly. In some aspects, the cisplatin is administered at a dose between about 40 mg/m² weekly and about 100 mg/m² weekly. In some aspects, the cisplatin is administered at a dose of about 40 mg/m² weekly. In some aspects, the cisplatin is administered at a dose of about 100 mg/m² weekly.

In some aspects, the platinum-based chemotherapeutic drug is carboplatin. In some aspects, the carboplatin is administered at a dose sufficient for the human patient to exhibit an area under the curve of about 1.0 to about 5.5 mg*min/mL. In some aspects, the carboplatin is administered at a dose sufficient for the human patient to exhibit an area under the curve of about 1.5 to about 2.0 mg*min/mL weekly. In some aspects, the carboplatin is administered at a dose sufficient for the human patient to exhibit an area under the curve of about 4.5 to about 5.0 every three weeks.

In some aspects, treatment may be initiated with administration of cisplatin and be continued with the administration of carboplatin in replacement of cisplatin.

In some aspects, the human patient is also administered radiation. In some aspects, the human patient is provided a suitable amount of radiation up to a maximum of 72 grays.

In some aspects, Compound A, or pharmaceutically acceptable salt thereof, is administered to the human patient after the patient has fasted for at least one hour. In some aspects, the human patient fasts for at least two hours after Compound A, or pharmaceutically acceptable salt thereof, is administered to the human patient.

In some aspects, the human patient receives Compound A, or pharmaceutically acceptable salt thereof, for the first 14 consecutive days of a 21 day cycle. In some aspects, the human patient receives the platinum-based chemotherapeutic drug on the second day of the 21 day cycle. In some aspects, the human patient receives Compound A, or pharmaceutically acceptable salt thereof, for up to three consecutive 21 day cycles. In some aspects, the human patient receives cisplatin on day 2 of the first 21 day cycle and carboplatin on day 2 of the second and/or third 21 day cycle.

In some aspects, the human patient receives five fractions of radiation each week for about 7 to about 9 weeks. In some aspects, the human patient receives Compound A, or pharmaceutically acceptable salt thereof, for 14 consecutive days after the last radiation dose.

In some aspects, the locally advanced squamous cell carcinoma is of the head and neck region. In some aspects, the locally advanced squamous cell carcinoma of the head and neck region is selected from the group consisting of oral cavity, oropharynx, larynx, and hypopharynx. In some aspects, the locally advanced squamous cell carcinoma of the head and neck region is oropharyngeal cancer. In some aspects, the oropharyngeal cancer is unrelated to human papillomavirus (HPV) infection (HPV negative). In some aspects, the oropharyngeal cancer is related to human papillomavirus (HPV) infection.

Methods of treating human patients having locally advanced squamous cell carcinoma comprising administering to the patient, in need thereof, Compound A, or pharmaceutically acceptable salt thereof, in an amount corresponding to about 100 mg to about 500 mg of Compound A as its free base, daily, wherein the patient in need thereof has a positive smoking history and/or is a heavy consumer of alcohol, wherein the method comprises:
(a) a first regimen comprising concomitant administration of Compound A, or pharmaceutically acceptable salt thereof, a platinum-based chemotherapeutic drug, and radiation;
(b) a second regimen comprising administration Compound A, or pharmaceutically acceptable salt thereof
are also provided.

In some aspects, the first regimen comprises a 21-day cycle in which the Compound A, or pharmaceutically acceptable salt thereof, and platinum-based chemotherapeutic drug are administered for 14 consecutive days followed by seven days in which no Compound A, or pharmaceutically acceptable salt thereof, or platinum-based chemotherapeutic drug are administered. In some aspects, the first regimen comprises three consecutive 21-day cycles.

In some aspects, the second regimen comprises a 21-day cycle in which the Compound A, or pharmaceutically acceptable salt thereof, is administered for 14 consecutive days followed by seven days in which no Compound A, or pharmaceutically acceptable salt thereof, is administered. In some aspects, the second regimen comprises two consecutive 21-day cycles. In some aspects, no platinum-based chemotherapeutic drug or radiation is administered in the second regimen.

In some aspects, the human patient has not previously received treatment (e.g., surgery, chemotherapy, radiotherapy, or immunotherapy) of the locally advanced squamous cell carcinoma.

In some aspects, the human patient is administered about 200 mg of Compound A, or pharmaceutically acceptable salt, based on its free base, in both the first and second regimens.

In some aspects, the human patient has a positive smoking history. In some aspects, the human patient is a heavy consumer of alcohol.

In some aspects, Compound A is in free base form. In some aspects, Compound A is a pharmaceutically acceptable salt. In some aspects, Compound A is a pharmaceutically acceptable salt form selected from the group consisting of acetate, hydrochloride, citrate, lactate, fumarate, succinate, phosphate, maleate, sulfate, tartrate, benzoate, mesylate, malate, hydrobromide, tosylate, nitrate, N-acetylglycine, ascorbate, butanate, ethane-1,2-disulfonate, gentisate, glucoronate, glutarate, glycolate, isethionate, ketoglutarate, malonate, napadisylate, napsylate, nicotinate, pyroglutamate, pyruvate, sebacate, and succinate. In some aspects, the pharmaceutically acceptable form is selected from the group consisting of sulfate, tosylate, gentisate, hydrochloride, napadisylate, napsylate, laurylsulfate, xinafoate, and pamoate.

In some aspects, Compound A, or pharmaceutically acceptable salt thereof, is administered orally. In some aspects, Compound A, or pharmaceutically acceptable salt thereof, is administered as a solid dosage form. In some aspects, the solid dosage form is a capsule or tablet.

In some aspects, Compound A, or pharmaceutically acceptable salt thereof, is administered as a solution. In some aspects, Compound A, or pharmaceutically acceptable salt thereof, is administered as an aqueous solution. In some aspects, Compound A, or pharmaceutically acceptable salt thereof, is administered orally via a nasogastric tube, percutaneous endoscopic gastrostomy tube, or percutaneous endoscopic jejunostomy tube. In some aspects, the solution (e.g., aqueous solution) comprises Compound A, or pharmaceutically acceptable salt thereof, at a concentration of between about 10 mg/mL to about 50 mg/mL based on Compound A as its free base. In some aspects, the solution (e.g., aqueous solution) comprises Compound A, or pharmaceutically acceptable salt thereof, at a concentration of about 20 mg/mL based on Compound A, as its free base.

In some aspects, Compound A, or pharmaceutically acceptable salt thereof, is administered as one dose one time per day. In some aspects, Compound A, or pharmaceutically acceptable salt thereof, is divided into multiple doses that are administered one, two, three, or four times per day.

In some aspects, the platinum-based chemotherapeutic drug of the first regimen is intravenously administered. In some aspects, the platinum-based chemotherapeutic drug of the first regimen is one or more selected from the group consisting of cisplatin, carboplatin, oxaliplatin, and nedaplatin.

In some aspects, the platinum-based chemotherapeutic drug of the first regimen is cisplatin. In some aspects, the cisplatin is administered at a dose between about 10 mg/m² weekly and 150 mg/m² weekly. In some aspects, the cisplatin is administered at a dose between about 40 mg/m² weekly and about 100 mg/m² weekly. In some aspects, the cisplatin is administered at a dose of about 40 mg/m² weekly. In some aspects, the cisplatin is administered at a dose of about 100 mg/m² weekly.

In some aspects, the platinum-based chemotherapeutic drug of the first regimen is carboplatin. In some aspects, the carboplatin is administered at a dose sufficient for the human patient to exhibit an area under the curve of about 1.0 to about 5.5 mg*min/mL. In some aspects, the carboplatin is administered at a dose sufficient for the human patient to exhibit an area under the curve of about 1.5 to about 2.0 mg*min/mL weekly. In some aspects, the carboplatin is administered at a dose sufficient for the human patient to exhibit an area under the curve of about 4.5 to about 5.0 every three weeks.

In some aspects, treatment may be initiated with administration of cisplatin and be continued with the administration of carboplatin in replacement of cisplatin.

In some aspects, the human patient is provided a suitable amount of radiation up to a maximum of 72 grays in the first regimen.

In some aspects, Compound A, or pharmaceutically acceptable salt thereof, is administered to the human patient in the first regimen after the patient has fasted for at least one hour. In some aspects, the human patient fasts for at least two hours after Compound A, or pharmaceutically acceptable salt thereof, is administered to the human patient in the first regimen.

In some aspects, Compound A, or pharmaceutically acceptable salt thereof, is administered to the human patient in the second regimen after the patient has fasted for at least one hour. In some aspects, the human patient fasts for at least two hours after Compound A, or pharmaceutically acceptable salt thereof, is administered to the human patient in the second regimen.

In some aspects, the locally advanced squamous cell carcinoma is inoperable. In some aspects, the locally advanced squamous cell carcinoma is Stage III, Stage IVA, or Stage IVB according to the American Joint Committee on Cancer 1998 staging system.

In some aspects, the locally advanced squamous cell carcinoma is of the head and neck region. In some aspects, the locally advanced squamous cell carcinoma of the head and neck region is inoperable. In some aspects, the locally advanced squamous cell carcinoma of the head and neck region is selected from the group consisting of oral cavity, oropharynx, larynx, and hypopharynx. In some aspects, the locally advanced squamous cell carcinoma of the head and neck region is oropharyngeal cancer. In some aspects, the oropharyngeal cancer is unrelated to human papillomavirus (HPV) infection (as determined by p16 immunohistochemistry (IHC). In some aspects, the oropharyngeal cancer is related to human papillomavirus (HPV) infection (as determined by p16 immunohistochemistry (IHC)). In some aspects, the locally advanced squamous cell carcinoma of the head and neck region is Stage III, Stage IVA, or Stage IVB according to the American Joint Committee on Cancer 1998 staging system.

Aspects of embodiment B are summarized in the following.

Methods for treating a human patient having locally advanced squamous cell carcinoma comprising administering to the human patient, in need thereof, one or more inhibitors of apoptosis protein (“IAP”) antagonists, wherein the methods comprise:
(a) a first regimen comprising concomitant administration of the one or more IAP antagonists, a platinum-based chemotherapeutic drug, and radiation; and
(b) a second regimen comprising administration of the one or more IAP antagonists, are provided herein.

In some aspects, the first regimen comprises at least one 21-day cycle in which the one or more IAP antagonists is administered for 14 consecutive days followed by seven days in which no IAP antagonist is administered. In some aspects, the first regimen comprises three consecutive 21-day cycles.

In some aspects, the platinum-based chemotherapeutic drug of the first regimen is administered on the second day of each 21-day cycle.

In some aspects, radiation is administered for five of seven days of each of the three weeks in a 21-day cycle. In some aspects, the radiation is administered for five of seven days in a week for a total of seven to nine consecutive weeks. In some aspects, the radiation is administered for five of seven days in a week for seven consecutive weeks. In some aspects, the radiation is administered for five of seven days in a week for eight consecutive weeks. In some aspects, the radiation is administered for five of seven days in a week for nine consecutive weeks.

In some aspects, radiation is administered on the first five consecutive days of each of the three weeks in a 21-day cycle. In some aspects, the radiation is administered on the first five consecutive days for seven to nine consecutive weeks. In some aspects, the radiation is administered on the first five consecutive days for seven consecutive weeks. In some aspects, the radiation is administered on the first five consecutive days for eight consecutive weeks. In some aspects, the radiation is administered on the first five consecutive days for nine consecutive weeks.

In some aspects, the radiation is intensity-modulated radiotherapy (“IMRT”).

In some aspects, the second regimen comprises at least one 21-day cycle in which at least one IAP antagonist is administered for 14 consecutive days followed by seven days in which no IAP antagonist is administered. In some aspects, the second regimen comprises two consecutive 21-day cycles. In some aspects, the second regimen comprises three consecutive 21-day cycles. In some aspects, no platinum-based chemotherapeutic drug or radiation is administered during the second regimen.

Methods for treating a human patient having locally advanced squamous cell carcinoma comprising administering to the human patient, in need thereof, one or more IAP antagonists, wherein the methods comprise:
(a) a first regimen comprising at least one 21-day cycle that comprises administering one or more IAP antagonists on Days 1-14 of the cycle, a platinum-based chemotherapeutic drug on Day 2 of the cycle, and radiation on Days 1-5, 8-12, and 15-19 of the cycle; and
(b) a second regimen comprising at least one 21-day cycle that comprises administering one or more IAP antagonists on Days 1-14 of the cycle
   are also provided herein. In some aspects, the first regimen comprises three consecutive 21-day cycles. In some aspects, radiation is administered on the first week of the third 21-day cycle. In some aspects, radiation is administered on the first and second weeks of the third 21-day cycle. In some aspects, radiation is administered on all three weeks of the third 21-day cycle.

In some aspects, the second regimen comprises three consecutive 21-day cycles. In some aspects, the second regimen comprises two consecutive 21-day cycles.

In some aspects, at least one IAP antagonist is selected from the group consisting of Compound A, or pharmaceutically acceptable salt thereof, LCL-161, or pharmaceutically acceptable salt thereof, CUDC 427/GDC 0917, or pharmaceutically acceptable salt thereof, birinapant, or pharmaceutically acceptable salt thereof, AZD5582, or pharmaceutically acceptable salt thereof, APG-1387, or pharmaceutically acceptable salt thereof, ASTX660, or pharmaceutically acceptable salt thereof, SBP-0636457, JP1201, or pharmaceutically acceptable salt thereof, or combinations thereof. In some aspects, the human patient is administered a first IAP antagonist that is Compound A, or pharmaceutically acceptable salt thereof, LCL-161, or pharmaceutically acceptable salt thereof, or CUDC 427/GDC 0917, and a second IAP antagonist selected from the group consisting of birinapant, or pharmaceutically acceptable salt thereof, AZD5582, or pharmaceutically acceptable salt thereof, and APG-1387, or pharmaceutically acceptable salt thereof, and combinations thereof. In some aspects, the human patient is administered a third IAP antagonist selected from the group consisting of ASTX660, or pharmaceutically acceptable salt thereof, SBP-0636457, JP1201, or pharmaceutically acceptable salt thereof, and combinations thereof.

In some aspects, at least one IAP antagonist is selected from the group consisting of monovalent IAP antagonists. This group includes especially Compound A, LCL-161 (Novartis, CAS No. 1005342-46-0), CUDC 427/GDC 0917 (Curis/Genentec, CAS No 1446182-94-0), ASTX660 (Astex, CAS No. 1799328-86-1), SBP-0636457 (Sandford Burnham Prebys Medical Discovery Institute, CAS No. 1422180-49-1) and pharmaceutically acceptable salts thereof.

In some aspects, at least one IAP antagonist is selected from the group consisting of bivalent IAP antagonists. This group includes especially TL-32711/Birinapant (Medivir, CAS No. 1260251-31-7), AZD5582 (AstraZeneca; CAS No. 1258392-53-8), APG-1387 (Ascentage Pharma, SM-1387, CAS No. 1570231-89-8), JP1201 (Joyant Pharmaceuticals) and pharmaceutically acceptable salts thereof.

The structures of some of the above IAP antagonists are shown in FIGS. 5 and 6 of Finlay D, Teriete P, Vamos M et al. “Inducing death in tumor cells: roles of the inhibitor of apoptosis proteins” [version 1; referees: 3 approved]. F1000Research 2017, 6(F1000 Faculty Rev):587 (https://doi.org/10.12688/f1000research.10625.1). IAP antagonists, for which it is unknown whether they are mono- or bivalent are also suitable according to other aspects. This group includes IAP inhibitors developed by Boehringer Ingelheim (see in WO 2013/127729, WO 2015/025018, WO 2015/025019, WO 2016/023858, or WO 2018/178250), in particular IAP inhibitor called BI 891065. Further suitable IAP Inhibitors of this aspect are described for instance in WO 2008/128171 A, WO 2014/031487 A, WO 2011/050068 A, WO 2008/014240 A, WO 2007/131366 A, WO 2007/130626 A, WO 2011/057099, WO 2009/140447, EP 2 698 158, WO 2008/014229 A, WO 2017/117684 A1, WO 2016/079527 A1 and WO 2018/178250 A1 and also in Table 1 of WO 2017/143449 A, which refers to these compounds as Smac mimetic compounds. All of such IAP inhibitors known from the literature including the article by T. W. Owens et al. in J Carcinog Mutagen. 2013 May 27; Suppl 14: S14-004 (Published online 2013 May 27. doi: [10.4172/2157-2518.S14-004]), and especially its Table 2, and literature cited therein, may also be used in the present aspect. Of course, it is also possible in this aspect to use a combination of two or more different IAP inhibitors. In this case, each IAP inhibitor may be selected independently from the available IAP inhibitors as described herein.

In some aspects, at least one IAP antagonist is Compound A, or pharmaceutically acceptable salt thereof. In some aspects, the human patient is administered at least one IAP antagonist that is Compound A, or pharmaceutically acceptable salt thereof, in an amount corresponding to about 100 mg to about 500 mg of Compound A as its free base, daily. In some aspects, the human patient is administered about 100 mg of Compound A, or pharmaceutically acceptable salt, based on its free base, daily. In some aspects, the human patient is administered about 150 mg of Compound A, or pharmaceutically acceptable salt, based on its free base, daily. In some aspects, the human patient is administered about 200 mg of Compound A, or pharmaceutically acceptable salt, based on its free base, daily. If, according to other aspects of the invention, another IAP antagonist is used, appropriate dosages may be derived from the literature, such as WO 2017/143449 A and D. Finlay et al. in F1000Research 2017, 6(F1000 Faculty Rev):587 (https://doi.org/10.12688/f1000research.10625.1) and references cited therein or, more specifically,
US 2013/005663 and WO 2016/054555 A for LCL-161;
US 2013/0005663 A for CUDC 427;
WO 2014/121178 A and US 2014/243276 A for birinapant;
WO 2010/142994 A for AZD5582;
Z. Chen et al. in Front Pharmacol. 2018; 9: 1298; doi: 10.3389/fphar.2018.01298 as well as B. Li et al. in J Exp Clin Cancer Res. 2018 Mar. 12; 37(1):53. doi: 10.1186/s13046-018-0703-9 and literature cited therein for APG-1387;
EP 3 083 616 A for ASTX660;
WO 2014/085489 A for SBP-0636457; and
WO 2011/059763 A for JP1201.
   Alternatively, suitable dosages may be determined by means of dose escalation studies.

In some aspects, the human patient has a positive smoking history, is a heavy consumer of alcohol, and/or has HPV-negative OPC. In some aspects, the human patient has a positive smoking history. In some aspects, the human patient is a heavy consumer of alcohol. In some aspects, the human patient has HPV-negative OPC.

In some aspects, at least one IAP antagonist is Compound A is in free base form. In some aspects, at least one IAP antagonist is Compound A is a pharmaceutically acceptable salt. In some aspects, Compound A is a pharmaceutically acceptable salt form selected from the group consisting of acetate, hydrochloride, citrate, lactate, fumarate, succinate, phosphate, maleate, sulfate, tartrate, benzoate, mesylate, malate, hydrobromide, tosylate, nitrate, N-acetylglycine, ascorbate, butanate, ethane-1,2-disulfonate, gentisate, glucoronate, glutarate, glycolate, isethionate, ketoglutarate, malonate, napadisylate, napsylate, nicotinate, pyroglutamate, pyruvate, sebacate, and succinate. In some aspects, the pharmaceutically acceptable form is selected from the group consisting of sulfate, tosylate, gentisate, hydrochloride, napadisylate, napsylate, laurylsulfate, xinafoate, and pamoate.

In some aspects, at least one IAP antagonist is Compound A, or pharmaceutically acceptable salt thereof, that is administered orally. In some aspects, at least one IAP antagonist is Compound A, or pharmaceutically acceptable salt thereof, that is administered as a solid dosage form. In some aspects, the solid dosage form is a capsule or tablet.

In some aspects, at least one IAP antagonist is Compound A, or pharmaceutically acceptable salt thereof, that is administered as a solution. In some aspects, the solution is an aqueous solution. In some aspects, the solution of Compound A, or pharmaceutically acceptable salt thereof, is administered orally via a nasogastric tube, percutaneous endoscopic gastrostomy tube, or percutaneous endoscopic jejunostomy tube. In some aspects, the solution comprises Compound A, or pharmaceutically acceptable salt thereof, at a concentration of between about 10 mg/mL to about 50 mg/mL based on Compound A as its free base. In some aspects, the solution comprises Compound A, or pharmaceutically acceptable salt thereof, at a concentration of about 20 mg/mL based on Compound A, as its free base.

In some aspects, at least one IAP antagonist is Compound A, or pharmaceutically acceptable salt thereof, that is administered as one dose one time per day. In some aspects, at least one IAP antagonist is Compound A, or pharmaceutically acceptable salt thereof, that is divided into multiple doses that are administered two, three, or four times per day.

In some aspects, the platinum-based chemotherapeutic drug of the first regimen is intravenously administered. In some aspects, the platinum-based chemotherapeutic drug of the first regimen is one or more selected from the group consisting of cisplatin, carboplatin, oxaliplatin, and nedaplatin.

In some aspects, the platinum-based chemotherapeutic drug of the first regimen is cisplatin. In some aspects, the cisplatin is administered at a dose between about 10 mg/m² weekly and about 150 mg/m² weekly. In some aspects, the cisplatin is administered at a dose between about 40 mg/m² weekly and about 100 mg/m² weekly. In some aspects, the cisplatin is administered at a dose of about 100 mg/m². In some aspects, the cisplatin is administered by slow intravenous infusion over at least 90 minutes. In some aspects, the cisplatin is administered at a dose of about 100 mg/m² by slow intravenous infusion over at least 90 minutes.

In some aspects, the platinum-based chemotherapeutic drug of the first regimen is carboplatin. In some aspects, the carboplatin is administered at a dose sufficient for the human patient to exhibit an area under the curve of about 1.0 to about 5.5 mg*min/mL. In some aspects, the carboplatin is administered at a dose sufficient for the human patient to exhibit an area under the curve of about 1.5 to about 2.0 mg*min/mL weekly. In some aspects, the carboplatin is administered at a dose sufficient for the human patient to exhibit an area under the curve of about 4.5 to about 5.0 every three weeks.

In some aspects, the human patient is provided a suitable amount of radiation up to a maximum of 70 grays in the first regimen. In some aspects, the human patient is provided a suitable amount of radiation up to a maximum of 56 grays. In some aspects, the radiation is IMRT.

In some aspects, at least one IAP antagonist is administered to the human patient in the first regimen early in the morning, after the human patient has fasted for at least two hours. In some aspects, the human patient fasts for at least one hour after at least one IAP antagonist is administered to the human patient in the first regimen. In some aspects, at least one IAP antagonist is administered to the human patient in the second regimen after the human patient has fasted for at least two hours. In some aspects, the human patient fasts for at least one hour after at least one IAP antagonist is administered to the human patient in the second regimen.

In some aspects, the locally advanced squamous cell carcinoma is of the head and neck region. In some aspects, the locally advanced squamous cell carcinoma of the head and neck region is selected from the group consisting of oropharynx, larynx, and hypopharynx. In some aspects, the locally advanced squamous cell carcinoma of the head and neck region is oropharyngeal cancer. In some aspects, the oropharyngeal cancer is unrelated to human papillomavirus (HPV) infection. In some aspects, the oropharyngeal cancer is related to human papillomavirus (HPV) infection. In some aspects, the locally advanced squamous cell carcinoma is Stage III, Stage IVA, or Stage IVB according to the American Joint Committee on Cancer 1998 staging system.

Methods for treating human patients having locally advanced squamous cell carcinoma comprising administering to the human patient, in need thereof, Compound A, or pharmaceutically acceptable salt thereof, in an amount corresponding to about 100 mg to about 500 mg of Compound A as its free base, daily, wherein the human patient in need thereof has a positive smoking history and/or is a heavy consumer of alcohol are further provided herein. In some aspects, the human patient has not previously received treatment (e.g., surgery, chemotherapy, radiotherapy, immunotherapy) of the locally advanced squamous cell carcinoma. In some aspects, the human patient is administered about 100 mg of Compound A, or pharmaceutically acceptable salt, based on its free base daily. In some aspects, the human patient is administered about 150 mg of Compound A, or pharmaceutically acceptable salt, based on its free base daily In some aspects, the human patient is administered about 200 mg of Compound A, or pharmaceutically acceptable salt, based on its free base daily.

In some aspects, the human patient has a positive smoking history. In some aspects, the human patient is a heavy consumer of alcohol. In some aspects, the human patient has HPV-negative OPC.

In some aspects, Compound A is in free base form. In some aspects, Compound A is a pharmaceutically acceptable salt. In some aspects, Compound A is a pharmaceutically acceptable salt form selected from the group consisting of acetate, hydrochloride, citrate, lactate, fumarate, succinate, phosphate, maleate, sulfate, tartrate, benzoate, mesylate, malate, hydrobromide, tosylate, nitrate, N-acetylglycine, ascorbate, butanate, ethane-1,2-disulfonate, gentisate, glucoronate, glutarate, glycolate, isethionate, ketoglutarate, malonate, napadisylate, napsylate, nicotinate, pyroglutamate, pyruvate, sebacate, and succinate. In some aspects, the pharmaceutically acceptable form is selected from the group consisting of sulfate, tosylate, gentisate, hydrochloride, napadisylate, napsylate, laurylsulfate, xinafoate, and pamoate.

In some aspects, Compound A, or pharmaceutically acceptable salt thereof, is administered orally. In some aspects, Compound A, or pharmaceutically acceptable salt thereof, is administered as a solid dosage form. In some aspects, the solid dosage form is a capsule or tablet.

In some aspects, Compound A, or pharmaceutically acceptable salt thereof, is administered as a solution. In some aspects, the solution is an aqueous solution. In some aspects, Compound A, or pharmaceutically acceptable salt thereof, is administered orally via a nasogastric tube, percutaneous endoscopic gastrostomy tube, or percutaneous endoscopic jejunostomy tube. In some aspects, the solution (e.g., aqueous solution) comprises Compound A, or pharmaceutically acceptable salt thereof, at a concentration of between about 10 mg/mL to about 50 mg/mL based on Compound A as its free base. In some aspects, the solution (e.g., aqueous solution) comprises Compound A, or pharmaceutically acceptable salt thereof, at a concentration of about 20 mg/mL based on Compound A, as its free base.

In some aspects, Compound A, or pharmaceutically acceptable salt thereof, is administered as one dose one time per day. In some aspects, Compound A, or pharmaceutically acceptable salt thereof, is divided into multiple doses that are administered one, two, three, or four times per day.

In some aspects, the human patient is also administered a platinum-based chemotherapeutic drug. In some aspects, the platinum-based chemotherapeutic drug is intravenously administered. In some aspects, the platinum-based chemotherapeutic drug is one or more selected from the group consisting of cisplatin, carboplatin, oxaliplatin, and nedaplatin.

In some aspects, the platinum-based chemotherapeutic drug is cisplatin. In some aspects, the cisplatin is administered at a dose between about 10 mg/m² weekly and about 150 mg/m² weekly. In some aspects, the cisplatin is administered at a dose between about 40 mg/m² weekly and about 100 mg/m² weekly. In some aspects, the cisplatin is administered at a dose of about 100 mg/m² weekly. In some aspects, the cisplatin is administered at a dose of about 100 mg/m². In some aspects, the cisplatin is administered by slow intravenous infusion over at least 90 minutes. In some aspects, the cisplatin is administered at a dose of about 100 mg/m² by slow intravenous infusion over at least 90 minutes.

In some aspects, the platinum-based chemotherapeutic drug is carboplatin. In some aspects, the carboplatin is administered at a dose sufficient for the human patient to exhibit an area under the curve of about 1.0 to about 5.5 mg*min/mL. In some aspects, the carboplatin is administered at a dose sufficient for the human patient to exhibit an area under the curve of about 1.5 to about 2.0 mg*min/mL weekly. In some aspects, the carboplatin is administered at a dose sufficient for the human patient to exhibit an area under the curve of about 4.5 to about 5.0 every three weeks.

In some aspects, treatment may be initiated with administration of cisplatin and be continued with the administration of carboplatin in replacement of cisplatin.

In some aspects, the human patient is also administered radiation. In some aspects, the human patient is provided a suitable amount of radiation up to a maximum of 70 grays. In some aspects, the human patient is provided a suitable amount of radiation up to a maximum of 50 grays. In some aspects, the radiation is IMRT.

In some aspects, Compound A, or pharmaceutically acceptable salt thereof, is administered to the human patient after the human patient has fasted for at least two hours. In some aspects, the human patient fasts for at least one hour after Compound A, or pharmaceutically acceptable salt thereof, is administered to the human patient.

In some aspects, the human patient receives Compound A, or pharmaceutically acceptable salt thereof, for the first 14 consecutive days of a 21 day cycle. In some aspects, the human patient receives the platinum-based chemotherapeutic drug on the second day of the 21 day cycle. In some aspects, the human patient receives Compound A, or pharmaceutically acceptable salt thereof, for up to three consecutive 21 day cycles. In some aspects, the human patient receives cisplatin on day 2 of the first 21 day cycle and carboplatin on day 2 of the second and/or third 21 day cycle.

In some aspects, the human patient receives five fractions of radiation each week for 7 to 9 weeks. In some aspects, the human patient receives Compound A, or pharmaceutically acceptable salt thereof, for 14 consecutive days after the last radiation dose.

In some aspects, the locally advanced squamous cell carcinoma is of the head and neck region. In some aspects, the locally advanced squamous cell carcinoma of the head and neck region is selected from the group consisting oropharynx, larynx, and hypopharynx. In some aspects, the locally advanced squamous cell carcinoma of the head and neck region is oropharyngeal cancer. In some aspects, the oropharyngeal cancer is unrelated to human papillomavirus (HPV) infection (HPV negative). In some aspects, the oropharyngeal cancer is related to human papillomavirus (HPV) infection.

Methods of treating human patients having locally advanced squamous cell carcinoma comprising administering to the human patient, in need thereof, Compound A, or pharmaceutically acceptable salt thereof, in an amount corresponding to about 100 mg to about 500 mg of Compound A as its free base, daily, wherein the human patient in need thereof has a positive smoking history, is a heavy consumer of alcohol, and/or has HPV-negative OPC, wherein the methods comprise:
(a) a first regimen comprising concomitant administration of Compound A, or pharmaceutically acceptable salt thereof, a platinum-based chemotherapeutic drug, and radiation;
(b) a second regimen comprising administration Compound A, or pharmaceutically acceptable salt thereof
are also provided.

In some aspects, the first regimen comprises at least one 21-day cycle in which the Compound A, or pharmaceutically acceptable salt thereof, and platinum-based chemotherapeutic drug are administered for 14 consecutive days followed by seven days in which no Compound A, or pharmaceutically acceptable salt thereof, or platinum-based chemotherapeutic drug are administered. In some aspects, the first regimen comprises three consecutive 21-day cycles.

In some aspects, the second regimen comprises at least one 21-day cycle in which the Compound A, or pharmaceutically acceptable salt thereof, is administered for 14 consecutive days followed by seven days in which no Compound A, or pharmaceutically acceptable salt thereof, is administered. In some aspects, the second regimen comprises two consecutive 21-day cycles. In some aspects, no platinum-based chemotherapeutic drug or radiation is administered in the second regimen.

In some aspects, the human patient has not previously received treatment (e.g., surgery, chemotherapy, radiotherapy, or immunotherapy) of the locally advanced squamous cell carcinoma.

In some aspects, the human patient is administered about 100 mg of Compound A, or pharmaceutically acceptable salt, daily, based on its free base, in both the first and second regimens. In some aspects, the human patient is administered about 150 mg of Compound A, or pharmaceutically acceptable salt, daily, based on its free base, in both the first and second regimens. In some aspects, the human patient is administered about 200 mg of Compound A, or pharmaceutically acceptable salt, daily, based on its free base, in both the first and second regimens.

In some aspects, the human patient has a positive smoking history. In some aspects, the human patient is a heavy consumer of alcohol. In some aspects, the human patient has HPV-negative OPC.

In some aspects, Compound A is in free base form. In some aspects, Compound A is a pharmaceutically acceptable salt. In some aspects, Compound A is a pharmaceutically acceptable salt form selected from the group consisting of acetate, hydrochloride, citrate, lactate, fumarate, succinate, phosphate, maleate, sulfate, tartrate, benzoate, mesylate, malate, hydrobromide, tosylate, nitrate, N-acetylglycine, ascorbate, butanate, ethane-1,2-disulfonate, gentisate, glucoronate, glutarate, glycolate, isethionate, ketoglutarate, malonate, napadisylate, napsylate, nicotinate, pyroglutamate, pyruvate, sebacate, and succinate. In some aspects, the pharmaceutically acceptable form is selected from the group consisting of sulfate, tosylate, gentisate, hydrochloride, napadisylate, napsylate, laurylsulfate, xinafoate, and pamoate.

In some aspects, Compound A, or pharmaceutically acceptable salt thereof, is administered orally. In some aspects, Compound A, or pharmaceutically acceptable salt thereof, is administered as a solid dosage form. In some aspects, the solid dosage form is a capsule or tablet.

In some aspects, Compound A, or pharmaceutically acceptable salt thereof, is administered as a solution. In some aspects, Compound A, or pharmaceutically acceptable salt thereof, is administered as an aqueous solution. In some aspects, Compound A, or pharmaceutically acceptable salt thereof, is administered orally via a nasogastric tube, percutaneous endoscopic gastrostomy tube, or percutaneous endoscopic jejunostomy tube. In some aspects, the solution (e.g., aqueous solution) comprises Compound A, or pharmaceutically acceptable salt thereof, at a concentration of between about 10 mg/mL to about 50 mg/mL based on Compound A as its free base. In some aspects, the solution (e.g., aqueous solution) comprises Compound A, or pharmaceutically acceptable salt thereof, at a concentration of about 20 mg/mL based on Compound A, as its free base.

In some aspects, Compound A, or pharmaceutically acceptable salt thereof, is administered as one dose one time per day. In some aspects, Compound A, or pharmaceutically acceptable salt thereof, is divided into multiple doses that are administered two, three, or four times per day.

In some aspects, the platinum-based chemotherapeutic drug of the first regimen is intravenously administered. In some aspects, the platinum-based chemotherapeutic drug of the first regimen is one or more selected from the group consisting of cisplatin, carboplatin, oxaliplatin, and nedaplatin.

In some aspects, the platinum-based chemotherapeutic drug of the first regimen is cisplatin. In some aspects, the cisplatin is administered at a dose between about 10 mg/m² weekly and 150 mg/m² weekly. In some aspects, the cisplatin is administered at a dose between about 40 mg/m² weekly and about 100 mg/m² weekly. In some aspects, the cisplatin is administered at a dose of about 100 mg/m². In some aspects, the cisplatin is administered by slow intravenous infusion over at least 90 minutes. In some aspects, the cisplatin is administered at a dose of about 100 mg/m² by slow intravenous infusion over at least 90 minutes.

In some aspects, the platinum-based chemotherapeutic drug of the first regimen is carboplatin. In some aspects, the carboplatin is administered at a dose sufficient for the human patient to exhibit an area under the curve of about 1.0 to about 5.5 mg*min/mL. In some aspects, the carboplatin is administered at a dose sufficient for the human patient to exhibit an area under the curve of about 1.5 to about 2.0 mg*min/mL weekly. In some aspects, the carboplatin is administered at a dose sufficient for the human patient to exhibit an area under the curve of about 4.5 to about 5.0 every three weeks.

In some aspects, treatment may be initiated with administration of cisplatin and be continued with the administration of carboplatin in replacement of cisplatin.

In some aspects, the human patient is provided a suitable amount of radiation up to a maximum of 72 grays in the first regimen. In some aspects, the human patient is provided a suitable amount of radiation up to a maximum of 70 grays in the first regimen. In some aspects, the human patient is provided a suitable amount of radiation up to a maximum of 50 grays in the first regimen. In some aspects, the radiation is IMRT.

In some aspects, Compound A, or pharmaceutically acceptable salt thereof, is administered to the human patient in the first regimen after the human patient has fasted for at least one hour. In some aspects, Compound A, or pharmaceutically acceptable salt thereof, is administered to the human patient in the first regimen after the human patient has fasted for at least two hours. In some aspects, the human patient fasts for at least one hour after Compound A, or pharmaceutically acceptable salt thereof, is administered to the human patient in the first regimen. In some aspects, the human patient fasts for at least two hours after Compound A, or pharmaceutically acceptable salt thereof, is administered to the human patient in the first regimen.

In some aspects, Compound A, or pharmaceutically acceptable salt thereof, is administered to the human patient in the second regimen after the human patient has fasted for at least one hour. In some aspects, Compound A, or pharmaceutically acceptable salt thereof, is administered to the human patient in the second regimen after the human patient has fasted for at least two hours. In some aspects, the human patient fasts for at least one hour after Compound A, or pharmaceutically acceptable salt thereof, is administered to the human patient in the second regimen. In some aspects, the human patient fasts for at least two hours after Compound A, or pharmaceutically acceptable salt thereof, is administered to the human patient in the second regimen.

In some aspects, the locally advanced squamous cell carcinoma is inoperable. In some aspects, the locally advanced squamous cell carcinoma is Stage III, Stage IVA, or Stage IVB according to the American Joint Committee on Cancer 1998 staging system.

In some aspects, the locally advanced squamous cell carcinoma is of the head and neck region. In some aspects, the locally advanced squamous cell carcinoma of the head and neck region is inoperable. In some aspects, the locally advanced squamous cell carcinoma of the head and neck region is selected from the group consisting of oral cavity, oropharynx, larynx, and hypopharynx. In some aspects, the locally advanced squamous cell carcinoma of the head and neck region is selected from the group consisting of, oropharynx, larynx, and hypopharynx. In some aspects, the locally advanced squamous cell carcinoma of the head and neck region is oropharyngeal cancer. In some aspects, the oropharyngeal cancer is unrelated to human papillomavirus (HPV) infection (as determined by p16 immunohistochemistry (HIC). In some aspects, the oropharyngeal cancer is related to human papillomavirus (HPV) infection (as determined by p16 immunohistochemistry (IHC). In some aspects, the locally advanced squamous cell carcinoma of the head and neck region is Stage III, Stage IVA, or Stage IVB according to the American Joint Committee on Cancer 1998 staging system.

### BRIEF DESCRIPTION OF DRAWINGS

FIG. 1A as filed with the priority application dated 25 Sep. 2019 (application text including examples corresponding to Examples 1 (variant 2), 2, 4 (variant 2), and 5 (variant 2) but not Example 3 and 6 of the present application) provides the Kaplan-Meier estimates of Progression-Free Survival.

FIG. 1B as filed with the priority application dated 28 Apr. 2020 (application text including examples corresponding to Examples 1 (variant 1), 2, 3, 4 (variant 1), 5 (variant 1) and 6 of the present application) provides the Kaplan-Meier estimates of Progression-Free Survival.

FIG. 1C provides the Kaplan-Meier estimates of Progression-Free Survival at 36 months of follow up, with censoring of late events (10 patients in this case, 5 in the Debio 1143 arm, 5 in the placebo arm) (late events are those occurring after missed assessments or scans—they are censored from PFS analysis at the last non-progressive disease (PD) assessment to avoid assumption of non-PD for long periods before PD is identified (according to FDA guidance).

FIG. 2A as filed with the priority application dated 25 Sep. 2019 provides the Kaplan-Meier estimates of locoregional control rate.

FIG. 2B as filed with the priority application dated 28 Apr. 2020 provides the Kaplan-Meier estimates of locoregional control rate.

FIG. 2C provides the Kaplan-Meier estimates of locoregional control rate over time from end of CRT (duration of locoregional control) at 36 months of follow up.

FIG. 3A as filed with the priority application dated 25 Sep. 2019 provides the Kaplan-Meier estimates of overall survival.

FIG. 3B as filed with the priority application dated 28 Apr. 2020 provides the Kaplan-Meier estimates of overall survival.

FIG. 3C provides the Kaplan-Meier estimates of overall survival at 36 months of follow up.

FIGS. 4A and B as filed with the priority application dated 25 Sep. 2019 report the secondary endpoints at 3 months and 6 months for Debio 1143+CRT (FIG. 4A) and placebo+CRT (FIG. 4B).

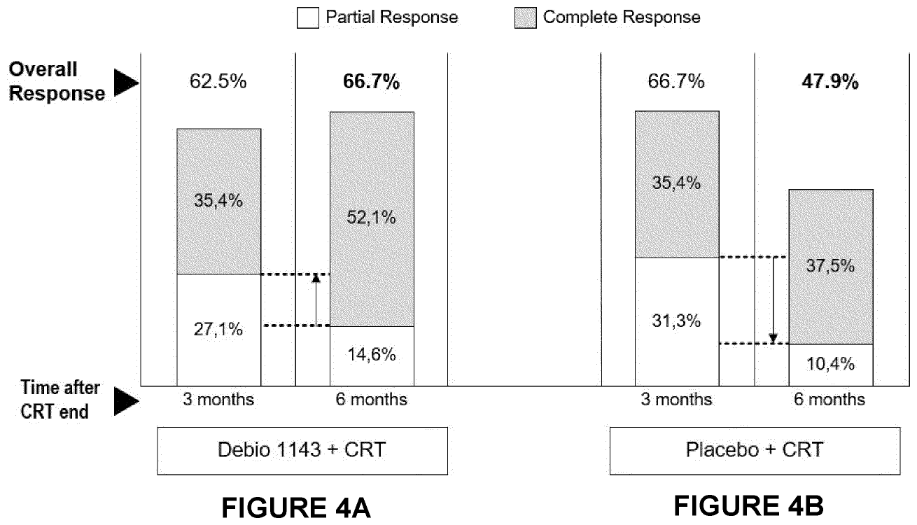

FIGS. 4C and D as filed with the priority application dated 28 Apr. 2020 report the secondary endpoints at 3 months and 6 months for Debio 1143+CRT (FIG. 4C) and placebo+CRT (FIG. 4D).

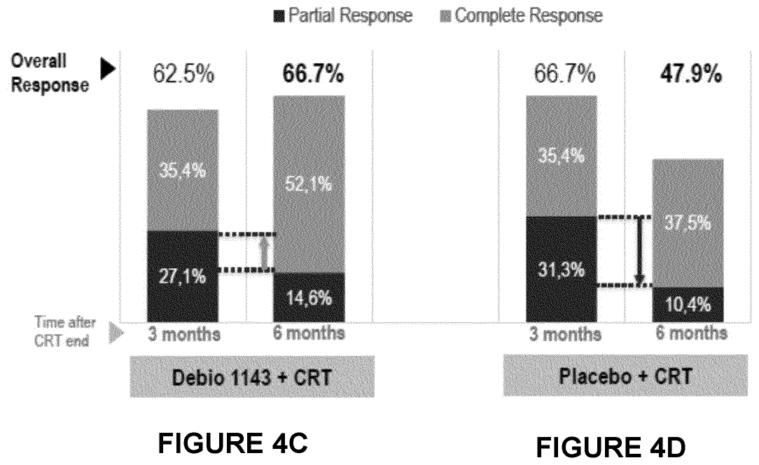

FIG. 5 provides a protocol for administration of an IAP antagonist (e.g. Compound A) in combination with a platinum-based chemotherapeutic agent and radiotherapy.

### DETAILED DESCRIPTION OF THE DISCLOSURE

The present disclosure provides methods of treating a human patient having locally advanced squamous cell carcinoma comprising administering to the human patient, in need thereof, an IAP antagonist (e.g., Compound A, or pharmaceutically acceptable salt thereof).

### I. Definitions

To facilitate an understanding of the present disclosure, a number of terms and phrases are defined below.

The term “Compound A” refers to (5S,8S,10aR)—N-benzhydryl-5-[[(2S)-2-(methylamino)propanoyl]amino]-3-(3-methylbutanoyl)-6-oxo-1,2,4,5,8,9,10,10a-octahydropyrrolo[1,2-a][1,5]diazocine-8-carboxamide or pharmaceutically acceptable salt thereof. This compound is also known as Debio 1143 or xevinapant.

The term “pharmaceutically acceptable salt” refers to a form of Compound A that consists of a cationic form of Compound A in combination with a suitable anion, or in the alternative, an anionic form of Compound A in combination with a cation.

The term “respond favorably” generally refers to causing a beneficial state in a subject. With respect to cancer treatment, the term refers to providing a therapeutic effect on the subject. Positive therapeutic effects in cancer can be measured in a number of ways (See, W. A. Weber, J. Nucl. Med. 50:1S-10S (2009)). For example, tumor growth inhibition, molecular marker expression, serum marker expression, and molecular imaging techniques can all be used to assess therapeutic efficacy of an anti-cancer therapeutic. A favorable response can be assessed, for example, by increased progression-free survival (PFS), disease-free survival (DFS), overall survival (OS), or metastasis-free survival (MFS), by complete response (CR), partial response (PR), or, in some cases, stable disease (SD), a decrease in progressive disease (PD), a reduced time to progression (TTP) or any combination thereof.

PFS, DFS, and OS can be measured by standards set by the National Cancer Institute and the U.S. Food and Drug Administration for the approval of new drugs. See Johnson et al, (2003) J. Clin. Oncol. 21(7):1404-1411.

“Progression free survival” (PFS) refers to the time from enrollment to disease progression or death. PFS is generally measured using the Kaplan-Meier method and Response Evaluation Criteria in Solid Tumors (RECIST) 1.1 standards. Generally, progression free survival refers to the situation wherein a patient remains alive, without the cancer getting worse.

“Time to Tumor Progression” (TTP) is defined as the time from enrollment to disease progression. TTP is generally measured using the RECIST 1.1 criteria.

A “complete response” or “complete remission” or “CR” indicates the disappearance of all signs of tumor or cancer in response to treatment. This does not always mean the cancer has been cured. For example, any pathological lymph nodes (whether target or non-target) must have reduction in the short axis to <10 mm. Complete response is generally measured using the RECIST 1.1 criteria. Eisenhauer, E. A., Eur. J. Cancer, 45:228-47 (2009).

A “partial response” or “PR” refers to a decrease in the size or volume of one or more tumors or lesions, or in the extent of cancer in the body, in response to treatment. Eisenhauer, E. A., Eur. J. Cancer, 45: 228-47 (2009).

“Stable disease” refers to disease without progression or relapse. In stable disease there is neither sufficient tumor shrinkage to qualify for partial response nor sufficient tumor increase to qualify as progressive disease taking as reference the smallest sum diameters while on the study. Eisenhauer, E. A., Eur. J. Cancer, 45: 228-47 (2009).

“Progressive disease” or “disease that has progressed” refers to the appearance of one more new lesions or tumors and/or the unequivocal progression of existing non-target lesions and/or at least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm. (Note: the appearance of one or more new lesions is also considered progression). Eisenhauer, E. A., Eur. J. Cancer, 45: 228-47 (2009).

“Overall Survival” (OS) refers to the time from patient enrollment to death or censored at the date last known alive. OS includes a prolongation in life expectancy as compared to naive or untreated individuals or patients. Overall survival refers to the situation wherein a patient remains alive for a defined period of time, such as one year, five years, etc., e.g., from the time of randomization or treatment.

Terms such as “treating” or “treatment” or “to treat” or “alleviating” or “to alleviate” refer to therapeutic measures that cure, slow down, lessen symptoms of, and/or halt progression of a diagnosed pathologic condition or disorder. Thus, those in need of treatment include those already diagnosed with or suspected of having the disorder. In some aspects, a subject is successfully “treated” for cancer, e.g., an advanced solid malignancy, according to the methods described herein if the human patient shows one or more of the following: a reduction in the number of or complete absence of cancer cells; a reduction in the tumor burden; inhibition of or an absence of cancer cell infiltration into peripheral organs including, for example, the spread of cancer into soft tissue and bone; inhibition of or an absence of tumor metastasis; inhibition or an absence of tumor growth; relief of one or more symptoms associated with the specific cancer; reduced morbidity and mortality; improvement in quality of life; reduction in tumorigenicity, tumorigenic frequency, or tumorigenic capacity, of a tumor; reduction in the number or frequency of cancer stem cells in a tumor; differentiation of tumorigenic cells to a non-tumorigenic state; increased progression-free survival (PFS), disease-free survival (DFS), or overall survival (OS), or metastasis-free survival (MFS), complete response (CR), partial response (PR), stable disease (SD), a decrease in progressive disease (PD), a reduced time to progression (TTP), or any combination thereof.

Prophylactic or preventative measures refer to measures that prevent and/or slow the development of a targeted pathological condition or disorder. Thus, those in need of prophylactic or preventative measures include those prone to have the disorder and those in whom the disorder is to be prevented.

As used in the present disclosure and claims, the singular forms “a,” “an,” and “the” include plural forms unless the context clearly dictates otherwise. In some aspects, disclosures of numerical values are to be understood as disclosures of the specified value ±10%. In other aspects, disclosures of numerical values are to be understood as disclosures of the specified value including possible variation as indicated by the specified degree of precision in combination with normal rounding rules. In yet other aspects, disclosures of numerical values are to be understood as disclosures of the specified value with no possible variation. In some embodiments, indications of two or more alternative numerical values are to be understood as disclosures of each and every of the numerical ranges spanned by the different combinations of two or the disclosed numerical values.

It is understood that wherever aspects are described herein with the language “comprising,” otherwise analogous aspects described in terms of “consisting of” and/or “consisting essentially of” are also provided. For instance, the wording “a second regimen comprising administration of the one or more IAP antagonists” refers in some aspects to a second regimen consisting of administration of the one or more IAP antagonists, while no platinum-based chemotherapeutic drug and no radiation is administered during this second regimen.

### II. Methods of Use

Methods of use according to embodiment A are described hereinbelow.

Compound A and its pharmaceutically acceptable salt thereof are useful in a variety of applications including, but not limited to, therapeutic treatment methods, such as the treatment of human patients having locally advanced squamous cell carcinoma. Methods for treating human patients having locally advanced squamous cell carcinoma comprising administering to the patient, in need thereof, Compound A, or pharmaceutically acceptable salt thereof, in an amount corresponding to about 100 mg to about 500 mg of Compound A as its free base, daily, wherein the patient in need thereof has a positive smoking history and/or is a heavy consumer of alcohol are provided herein.

In some aspects, the human patient is administered about 100 mg to about 450 mg, about 100 mg to about 400 mg, about 100 mg to about 350 mg, about 150 mg to about 300 mg, or about 200 mg to about 250 mg of Compound A, or pharmaceutically acceptable salt, based on its free base. In some aspects, the human patient is administered about 100 mg, about 150 mg, about 200 mg, about 250 mg, about 300 mg, about 400 mg, about 450 mg, or about 500 mg of Compound A, or pharmaceutically acceptable salt, based on its free base. In some aspects, the human patient is administered about 200 mg of Compound A, or pharmaceutically acceptable salt, based on its free base.

In some aspects, the human patient is also administered a platinum-based chemotherapeutic drug. In some aspects, the platinum-based chemotherapeutic drug is intravenously administered. In some aspects, the platinum-based chemotherapeutic drug is one or more selected from the group consisting of cisplatin, carboplatin, oxaliplatin, and nedaplatin.

In some aspects, the platinum-based chemotherapeutic drug is cisplatin. In some aspects, the cisplatin is administered at a dose between about 10 mg/m² weekly and 150 mg/m² weekly. In some aspects, the cisplatin is administered at a dose between about 20 mg/m² weekly and 125 mg/m² weekly. In some aspects, the cisplatin is administered at a dose between about 30 mg/m² weekly and 100 mg/m² weekly. In some aspects, the cisplatin is administered at a dose between about 40 mg/m² weekly and about 100 mg/m² weekly. In some aspects, the cisplatin is administered at a dose between about 50 mg/m² weekly and 100 mg/m² weekly. In some aspects, the cisplatin is administered at a dose between about 60 mg/m² weekly and 100 mg/m² weekly. In some aspects, the cisplatin is administered at a dose between about 70 mg/m² weekly and 100 mg/m² weekly. In some aspects, the cisplatin is administered at a dose of about 10 mg/m², 20 mg/m², 30 mg/m², 40 mg/m², 50 mg/m², 60 mg/m², 70 mg/m², 80 mg/m², 90 mg/m², or 100 mg/m² weekly.

In some aspects, the platinum-based chemotherapeutic drug is carboplatin. In some aspects, the carboplatin is administered at a dose sufficient for the human patient to exhibit an area under the curve of about 1.0 to about 5.5 mg*min/mL. In some aspects, the platinum-based chemotherapeutic drug is carboplatin. In some aspects, the carboplatin is administered at a dose sufficient for the human patient to exhibit an area under the curve of about 1.0 to about 5.0 mg*min/mL. In some aspects, the platinum-based chemotherapeutic drug is carboplatin. In some aspects, the carboplatin is administered at a dose sufficient for the human patient to exhibit an area under the curve of about 1.0 to about 4.5 mg*min/mL. In some aspects, the platinum-based chemotherapeutic drug is carboplatin. In some aspects, the carboplatin is administered at a dose sufficient for the human patient to exhibit an area under the curve of about 1.0 to about 4.0 mg*min/mL. In some aspects, the platinum-based chemotherapeutic drug is carboplatin. In some aspects, the carboplatin is administered at a dose sufficient for the human patient to exhibit an area under the curve of about 1.0 to about 3.5 mg*min/mL. In some aspects, the platinum-based chemotherapeutic drug is carboplatin. In some aspects, the carboplatin is administered at a dose sufficient for the human patient to exhibit an area under the curve of about 1.0 to about 3.0 mg*min/mL. In some aspects, the platinum-based chemotherapeutic drug is carboplatin. In some aspects, the carboplatin is administered at a dose sufficient for the human patient to exhibit an area under the curve of about 1.0 to about 2.5 mg*min/mL. In some aspects, the platinum-based chemotherapeutic drug is carboplatin. In some aspects, the carboplatin is administered at a dose sufficient for the human patient to exhibit an area under the curve of about 1.0 to about 2.0 mg*min/mL. In some aspects, the carboplatin is administered at a dose sufficient for the human patient to exhibit an area under the curve of about 1.5 to about 2.0 mg*min/mL weekly.

In some aspects, the carboplatin is administered at a dose sufficient for the human patient to exhibit an area under the curve of about 3.0 to about 5.5 every three weeks. In some aspects, the carboplatin is administered at a dose sufficient for the human patient to exhibit an area under the curve of about 3.5 to about 5.5 every three weeks. In some aspects, the carboplatin is administered at a dose sufficient for the human patient to exhibit an area under the curve of about 4.0 to about 5.5 every three weeks. In some aspects, the carboplatin is administered at a dose sufficient for the human patient to exhibit an area under the curve of about 4.5 to about 5.0 every three weeks.

In some aspects, treatment may be initiated with administration of cisplatin and be continued with the administration of carboplatin in replacement of cisplatin.

In some aspects, the human patient is also administered radiation. In some aspects, the human patient is provided a suitable amount of radiation up to a maximum of 100 grays. In some aspects, the human patient is provided a suitable amount of radiation up to a maximum of 85 grays. In some aspects, the human patient is provided a suitable amount of radiation up to a maximum of 72 grays.

In some aspects, Compound A, or pharmaceutically acceptable salt thereof, is administered to the human patient after the patient has fasted for at least one hour. In some aspects, the human patient fasts for at least two hours after Compound A, or pharmaceutically acceptable salt thereof, is administered to the human patient.

In some aspects, the human patient receives Compound A, or pharmaceutically acceptable salt thereof, for the first 14 consecutive days of a 21 day cycle. In some aspects, the human patient receives the platinum-based chemotherapeutic drug on the second day of the 21 day cycle. In some aspects, the human patient receives Compound A, or pharmaceutically acceptable salt thereof, for up to three consecutive 21 day cycles. In some aspects, the human patient receives cisplatin on day 2 of the first 21 day cycle and carboplatin on day 2 of the second and/or third 21 day cycle.

In some aspects, the human patient receives five fractions of radiation each week for about 7 to about 9 weeks. In some aspects, the human patient receives Compound A, or pharmaceutically acceptable salt thereof, for 14 consecutive days after the last radiation dose.

Methods of treating human patients having locally advanced squamous cell carcinoma comprising administering to the patient, in need thereof, Compound A, or pharmaceutically acceptable salt thereof, in an amount corresponding to about 100 mg to about 500 mg of Compound A as its free base, daily, wherein the patient in need thereof has a positive smoking history and/or is a heavy consumer of alcohol, wherein the method comprises:
(c) a first regimen comprising concomitant administration of Compound A, or pharmaceutically acceptable salt thereof, a platinum-based chemotherapeutic drug, and radiation;
(d) a second regimen comprising administration Compound A, or pharmaceutically acceptable salt thereof
   are also provided.

In some aspects, the first regimen comprises a 21-day cycle in which the Compound A, or pharmaceutically acceptable salt thereof, and platinum-based chemotherapeutic drug are administered for 14 consecutive days followed by seven days in which no Compound A, or pharmaceutically acceptable salt thereof, or platinum-based chemotherapeutic drug are administered. In some aspects, the first regimen comprises three consecutive 21-day cycles.

In some aspects, the second regimen comprises a 21-day cycle in which the Compound A, or pharmaceutically acceptable salt thereof, is administered for 14 consecutive days followed by seven days in which no Compound A, or pharmaceutically acceptable salt thereof, is administered. In some aspects, the second regimen comprises two consecutive 21-day cycles. In some aspects, no platinum-based chemotherapeutic drug or radiation is administered in the second regimen.

In some aspects, the human patient is administered about 100 mg to about 450 mg, about 100 mg to about 400 mg, about 100 mg to about 350 mg, about 150 mg to about 300 mg, or about 200 mg to about 250 mg of Compound A, or pharmaceutically acceptable salt, based on its free base. In some aspects, the human patient is administered about 100 mg, about 150 mg, about 200 mg, about 250 mg, about 300 mg, about 400 mg, about 450 mg, or about 500 mg of Compound A, or pharmaceutically acceptable salt, based on its free base. In some aspects, the human patient is administered about 200 mg of Compound A, or pharmaceutically acceptable salt, based on its free base.

In some aspects, the human patient is administered a different amount of Compound A, or pharmaceutically acceptable salt, based on its free base, in the first and second regimens. In some aspects, the human patient is administered a higher dose of Compound A, or pharmaceutically acceptable salt, in the first regimen than in the second regimen. In some aspects, the human patient is administered a higher dose of Compound A, or pharmaceutically acceptable salt in the second regimen than in the first regimen. In some aspects, the human patient is administered the same dose of Compound A, or pharmaceutically acceptable salt, in both the first and second regimens. In some aspects, the human patient is administered about 200 mg of Compound A, or pharmaceutically acceptable salt, based on its free base, in both the first and second regimens.

In some aspects, the platinum-based chemotherapeutic drug is administered intravenously. In some aspects, the platinum-based chemotherapeutic drug is one or more selected from the group consisting of cisplatin, carboplatin, oxaliplatin, and nedaplatin.

In some aspects, the platinum-based chemotherapeutic drug is cisplatin. In some aspects, the cisplatin is administered at a dose between about 10 mg/m² weekly and 150 mg/m² weekly. In some aspects, the cisplatin is administered at a dose between about 20 mg/m2 weekly and 125 mg/m² weekly. In some aspects, the cisplatin is administered at a dose between about 30 mg/m² weekly and 100 mg/m² weekly. In some aspects, the cisplatin is administered at a dose between about 40 mg/m² weekly and about 100 mg/m² weekly. In some aspects, the cisplatin is administered at a dose between about 50 mg/m² weekly and 100 mg/m² weekly. In some aspects, the cisplatin is administered at a dose between about 60 mg/m² weekly and 100 mg/m² weekly. In some aspects, the cisplatin is administered at a dose between about 70 mg/m² weekly and 100 mg/m² weekly. In some aspects, the cisplatin is administered at a dose of about 10 mg/m², 20 mg/m², 30 mg/m², 40 mg/m², 50 mg/m², 60 mg/m², 70 mg/m², 80 mg/m², 90 mg/m², or 100 mg/m² weekly.

In some aspects, the platinum-based chemotherapeutic drug is carboplatin. In some aspects, the carboplatin is administered at a dose sufficient for the human patient to exhibit an area under the curve of about 1.0 to about 5.5 mg*min/mL. In some aspects, the platinum-based chemotherapeutic drug is carboplatin. In some aspects, the carboplatin is administered at a dose sufficient for the human patient to exhibit an area under the curve of about 1.0 to about 5.0 mg*min/mL. In some aspects, the platinum-based chemotherapeutic drug is carboplatin. In some aspects, the carboplatin is administered at a dose sufficient for the human patient to exhibit an area under the curve of about 1.0 to about 4.5 mg*min/mL. In some aspects, the platinum-based chemotherapeutic drug is carboplatin. In some aspects, the carboplatin is administered at a dose sufficient for the human patient to exhibit an area under the curve of about 1.0 to about 4.0 mg*min/mL. In some aspects, the platinum-based chemotherapeutic drug is carboplatin. In some aspects, the carboplatin is administered at a dose sufficient for the human patient to exhibit an area under the curve of about 1.0 to about 3.5 mg*min/mL. In some aspects, the platinum-based chemotherapeutic drug is carboplatin. In some aspects, the carboplatin is administered at a dose sufficient for the human patient to exhibit an area under the curve of about 1.0 to about 3.0 mg*min/mL. In some aspects, the platinum-based chemotherapeutic drug is carboplatin. In some aspects, the carboplatin is administered at a dose sufficient for the human patient to exhibit an area under the curve of about 1.0 to about 2.5 mg*min/mL. In some aspects, the platinum-based chemotherapeutic drug is carboplatin. In some aspects, the carboplatin is administered at a dose sufficient for the human patient to exhibit an area under the curve of about 1.0 to about 2.0 mg*min/mL. In some aspects, the carboplatin is administered at a dose sufficient for the human patient to exhibit an area under the curve of about 1.5 to about 2.0 mg*min/mL weekly.

In some aspects, the carboplatin is administered at a dose sufficient for the human patient to exhibit an area under the curve of about 3.0 to about 5.5 every three weeks. In some aspects, the carboplatin is administered at a dose sufficient for the human patient to exhibit an area under the curve of about 3.5 to about 5.5 every three weeks. In some aspects, the carboplatin is administered at a dose sufficient for the human patient to exhibit an area under the curve of about 4.0 to about 5.5 every three weeks. In some aspects, the carboplatin is administered at a dose sufficient for the human patient to exhibit an area under the curve of about 4.5 to about 5.0 every three weeks.

In some aspects, treatment may be initiated with administration of cisplatin and be continued with the administration of carboplatin in replacement of cisplatin.

In some aspects, the human patient is also administered radiation. In some aspects, the human patient is provided a suitable amount of radiation up to a maximum of 100 grays. In some aspects, the human patient is provided a suitable amount of radiation up to a maximum of 85 grays. In some aspects, the human patient is provided a suitable amount of radiation up to a maximum of 72 grays.

In some aspects, Compound A, or pharmaceutically acceptable salt thereof, is administered to the human patient in the first regimen after the patient has fasted for at least one hour. In some aspects, the human patient fasts for at least two hours after Compound A, or pharmaceutically acceptable salt thereof, is administered to the human patient in the first regimen.

In some aspects, Compound A, or pharmaceutically acceptable salt thereof, is administered to the human patient in the second regimen after the patient has fasted for at least one hour. In some aspects, the human patient fasts for at least two hours after Compound A, or pharmaceutically acceptable salt thereof, is administered to the human patient in the second regimen.

Methods of use according to embodiment B are described hereinbelow.

Methods for treating a human patient having locally advanced squamous cell carcinoma comprising administering to the human patient, in need thereof, one or more inhibitors of apoptosis protein (“IAP”) antagonists, wherein the methods comprise:
(a) a first regimen comprising concomitant administration of the one or more IAP antagonists, a platinum-based chemotherapeutic drug, and radiation; and
(b) a second regimen comprising administration of the one or more IAP antagonists
   are provided herein. In some aspects, the first regimen comprises at least one 21-day cycle in which the one or more IAP antagonists is administered for 14 consecutive days followed by seven days in which no IAP antagonist is administered. In some aspects, the first regimen comprises three consecutive 21-day cycles.

In some aspects, the platinum-based chemotherapeutic drug of the first regimen is administered on the second day of each 21-day cycle.

In some aspects, the second regimen comprises at least one 21-day cycle in which at least one IAP antagonist is administered for 14 consecutive days followed by seven days in which no IAP antagonist is administered. In some aspects, the second regimen comprises two consecutive 21-day cycles. In some aspects, the second regimen comprises three consecutive 21-day cycles. In some aspects, no platinum-based chemotherapeutic drug or radiation is administered in the second regimen.

Methods for treating a human patient having locally advanced squamous cell carcinoma comprising administering to the human patient, in need thereof, one or more IAP antagonists, wherein the methods comprise:
(a) a first regimen comprising a 21-day cycle that comprises administering one or more IAP antagonists on Days 1-14 of the cycle, a platinum-based chemotherapeutic drug on Day 2 of the cycle, and radiation on Days 1-5, 8-12, and 15-19 of the cycle; and
(b) a second regimen comprising a 21-day cycle that comprises administering one or more IAP antagonists on Days 1-14 of the cycle
   are also provided herein. In some aspects, the first regimen comprises three consecutive 21-day cycles. In some aspects, second regimen comprises three consecutive 21-day cycles.

In some aspects, no platinum-based chemotherapeutic drug or radiation is administered in the second regimen.

In some aspects of the methods, the human patient has a positive smoking history and/or is a heavy consumer of alcohol. In some aspects, the human patient has a positive smoking history. In some aspects, the human patient is a heavy consumer of alcohol. In some patients, the human patient has HPV-negative OPC.

In some aspects, the one or more IAP antagonists is selected from the group consisting of birinapant, or pharmaceutically acceptable salt thereof, AZD5582, or pharmaceutically acceptable salt thereof, APG-1387, or pharmaceutically acceptable salt thereof, ASTX660, or pharmaceutically acceptable salt thereof, SBP-0636457, JP1201, or pharmaceutically acceptable salt thereof, or combinations thereof. In some aspects, at least one IAP antagonist is Compound A, or pharmaceutically acceptable salt thereof. In some aspects, the human patient is administered a first IAP antagonist that is Compound A, or pharmaceutically acceptable salt thereof, and a second IAP antagonist selected from the group consisting of birinapant, or pharmaceutically acceptable salt thereof, AZD5582, or pharmaceutically acceptable salt thereof, and APG-1387, or pharmaceutically acceptable salt thereof, and combinations thereof. In some aspects, the human patient is administered a third IAP antagonist selected from the group consisting of ASTX660, or pharmaceutically acceptable salt thereof, SBP-0636457, JP1201, or pharmaceutically acceptable salt thereof, and combinations thereof. The one or more IAP antagonists can be administered simultaneously or at different times during either the day or during the treatment regimen.

In some aspects, the human patient is administered at least one IAP antagonist that is Compound A, or pharmaceutically acceptable salt thereof. In some aspects, the human patient is administered about 100 mg to about 500 mg of Compound A, or pharmaceutically acceptable salt thereof, based on its free base daily. In some aspects, the human patient is administered about 100 mg to about 450 mg, about 100 mg to about 400 mg, about 100 mg to about 350 mg, about 150 mg to about 300 mg, or about 200 mg to about 250 mg of Compound A, or pharmaceutically acceptable salt, based on its free base daily. In some aspects, the human patient is administered about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, or about 500 mg of Compound A, or pharmaceutically acceptable salt, based on its free base daily. In some aspects, the human patient is administered about 100 mg of Compound A, or pharmaceutically acceptable salt, based on its free base daily. In some aspects, the human patient is administered about 150 mg of Compound A, or pharmaceutically acceptable salt, based on its free base, daily. In some aspects, the human patient is administered about 200 mg of Compound A, or pharmaceutically acceptable salt, based on its free base daily.

In some aspects, the human patient is also administered a platinum-based chemotherapeutic drug. In some aspects, the platinum-based chemotherapeutic drug is intravenously administered. In some aspects, the platinum-based chemotherapeutic drug is one or more selected from the group consisting of cisplatin, carboplatin, oxaliplatin, and nedaplatin.

In some aspects, the platinum-based chemotherapeutic drug is cisplatin. In some aspects, the cisplatin is administered at a dose between about 10 mg/m² weekly and about 150 mg/m² weekly. In some aspects, the cisplatin is administered at a dose between about 20 mg/m² weekly and about 125 mg/m² weekly. In some aspects, the cisplatin is administered at a dose between about 30 mg/m² weekly and about 100 mg/m² weekly. In some aspects, the cisplatin is administered at a dose between about 40 mg/m² weekly and about 100 mg/m² weekly. In some aspects, the cisplatin is administered at a dose between about 50 mg/m² weekly and about 100 mg/m² weekly. In some aspects, the cisplatin is administered at a dose between about 60 mg/m² weekly and about 100 mg/m² weekly. In some aspects, the cisplatin is administered at a dose between about 70 mg/m² weekly and about 100 mg/m² weekly. In some aspects, the cisplatin is administered at a dose of about 10 mg/m², about 20 mg/m², about 30 mg/m², about 40 mg/m², about 50 mg/m², about 60 mg/m², about 70 mg/m², about 80 mg/m², about 90 mg/m², or about 100 mg/m² weekly. In some aspects, the cisplatin is administered at a dose of about 100 mg/m².

In some aspects, the cisplatin is administered by slow intravenous infusion. In some aspects, the cisplatin is administered by slow intravenous infusion over at least 45 minutes, at least 60 minutes, at least 90 minutes, or at least 120 minutes.

In some aspects, the cisplatin is administered at a dose of about 100 mg/m² by slow intravenous infusion over at least 90 minutes.

In some aspects, the platinum-based chemotherapeutic drug is carboplatin. In some aspects, the carboplatin is administered at a dose sufficient for the human patient to exhibit an area under the curve of about 1.0 to about 5.5 mg*min/mL. In some aspects, the carboplatin is administered at a dose sufficient for the human patient to exhibit an area under the curve of about 1.0 to about 5.0 mg*min/mL. In some aspects, the carboplatin is administered at a dose sufficient for the human patient to exhibit an area under the curve of about 1.0 to about 4.5 mg*min/mL. In some aspects, the carboplatin is administered at a dose sufficient for the human patient to exhibit an area under the curve of about 1.0 to about 4.0 mg*min/mL. In some aspects, the carboplatin is administered at a dose sufficient for the human patient to exhibit an area under the curve of about 1.0 to about 3.5 mg*min/mL. In some aspects, the carboplatin is administered at a dose sufficient for the human patient to exhibit an area under the curve of about 1.0 to about 3.0 mg*min/mL. In some aspects, the carboplatin is administered at a dose sufficient for the human patient to exhibit an area under the curve of about 1.0 to about 2.5 mg*min/mL. In some aspects, the carboplatin is administered at a dose sufficient for the human patient to exhibit an area under the curve of about 1.0 to about 2.0 mg*min/mL. In some aspects, the carboplatin is administered at a dose sufficient for the human patient to exhibit an area under the curve of about 1.5 to about 2.0 mg*min/mL weekly.

In some aspects, the carboplatin is administered at a dose sufficient for the human patient to exhibit an area under the curve of about 3.0 to about 5.5 every three weeks. In some aspects, the carboplatin is administered at a dose sufficient for the human patient to exhibit an area under the curve of about 3.5 to about 5.5 every three weeks. In some aspects, the carboplatin is administered at a dose sufficient for the human patient to exhibit an area under the curve of about 4.0 to about 5.5 every three weeks. In some aspects, the carboplatin is administered at a dose sufficient for the human patient to exhibit an area under the curve of about 4.5 to about 5.0 every three weeks.

In some aspects, treatment may be initiated with administration of cisplatin and be continued with the administration of carboplatin in replacement of cisplatin.

In some aspects, the human patient is also administered radiation. In some aspects, the human patient is provided a suitable amount of radiation up to a maximum of 70 grays. In some aspects, the human patient is provided a suitable amount of radiation up to a maximum of 50 grays.

In some aspects, radiation is administered for five of seven days of each of the three weeks in a 21-day cycle. In some aspects, the radiation is administered for five of seven days in a week for a total of seven to nine consecutive weeks. In some aspects, the radiation is administered for five of seven days in a week for seven consecutive weeks. In some aspects, the radiation is administered for five of seven days in a week for eight consecutive weeks. In some aspects, the radiation is administered for five of seven days in a week for nine consecutive weeks.

In some aspects, radiation is administered on the first five consecutive days of each of the three weeks in a 21-day cycle. In some aspects, the radiation is administered on the first five consecutive days for seven to nine consecutive weeks. In some aspects, the radiation is administered on the first five consecutive days for seven consecutive weeks. In some aspects, the radiation is administered on the first five consecutive days for eight consecutive weeks. In some aspects, the radiation is administered on the first five consecutive days for nine consecutive weeks.

In some aspects, radiation should be delivered over seven weeks as follows:
a. To the gross-tumor volume (“GTV”) and high-risk clinical target volume: 5 fractions per week (5/7 days), 1 fraction per day, 2.0 grays/fraction, up to a total of 70 grays;
b. As elective irradiation (low-risk) to locoregional areas: 5 fractions per week (5/7 days), 1 fraction per day, 1.6 grays/fraction per day up to 56 grays.

In some aspects, the radiation is intensity-modulated radiotherapy (“IMRT”).

In some aspects, at least one IAP antagonist (e.g., Compound A, or pharmaceutically acceptable salt thereof), is administered to the human patient after the human patient has fasted for at least two hours. In some aspects, the human patient fasts for at least one hour after at least one IAP antagonist (e.g. Compound A, or pharmaceutically acceptable salt thereof), is administered to the human patient.

In some aspects, the human patient receives at least one IAP antagonist (e.g. Compound A, or pharmaceutically acceptable salt thereof), for the first 14 consecutive days of a 21 day cycle. In some aspects, the human patient receives the platinum-based chemotherapeutic drug on the second day of the 21 day cycle. In some aspects, the human patient receives at least one IAP antagonist (e.g., Compound A, or pharmaceutically acceptable salt thereof), for up to three consecutive 21 day cycles. In some aspects, the human patient receives cisplatin on day 2 of the first 21 day cycle and carboplatin on day 2 of the second and/or third 21 day cycle.

In some aspects, the human patient receives five fractions of radiation each week for 7 to 9 weeks. In some aspects, the human patient receives at least one IAP antagonist (e.g., Compound A, or pharmaceutically acceptable salt thereof), for 14 consecutive days after the last radiation dose.

Methods of treating human patients having locally advanced squamous cell carcinoma comprising administering to the human patient, in need thereof, one or more IAP antagonists (e.g., Compound A, or pharmaceutically acceptable salt thereof), wherein the human patient in need thereof has a positive smoking history and/or is a heavy consumer of alcohol, wherein the methods comprise:
(e) a first regimen comprising concomitant administration of one or more IAP antagonists (e.g., Compound A, or pharmaceutically acceptable salt thereof), a platinum-based chemotherapeutic drug, and radiation;
(f) a second regimen comprising administration the one or more IAP antagonists (e.g., Compound A, or pharmaceutically acceptable salt thereof)
are also provided.

In some aspects, the first regimen comprises at least one 21-day cycle in which at least one IAP antagonist (e.g., Compound A, or pharmaceutically acceptable salt thereof), and platinum-based chemotherapeutic drug are administered for 14 consecutive days followed by seven days in which no IAP antagonist or platinum-based chemotherapeutic drug are administered. In some aspects, the first regimen comprises three consecutive 21-day cycles.

In some aspects, the second regimen comprises at least one 21-day cycle in which at least one IAP antagonist is administered for 14 consecutive days followed by seven days in which no IAP antagonist is administered. In some aspects, the second regimen comprises two consecutive 21-day cycles. In some aspects, no platinum-based chemotherapeutic drug or radiation is administered in the second regimen.

In some aspects, the one or more IAP antagonists is selected from the group consisting of birinapant, or pharmaceutically acceptable salt thereof, AZD5582, or pharmaceutically acceptable salt thereof, APG-1387, or pharmaceutically acceptable salt thereof, ASTX660, or pharmaceutically acceptable salt thereof, SBP-0636457, JP1201, or pharmaceutically acceptable salt thereof, or combinations thereof. In some aspects, at least one IAP antagonist is Compound A, or pharmaceutically acceptable salt thereof. In some aspects, the human patient is administered a first IAP antagonist that is Compound A, or pharmaceutically acceptable salt thereof, and a second IAP antagonist selected from the group consisting of birinapant, or pharmaceutically acceptable salt thereof, AZD5582, or pharmaceutically acceptable salt thereof, and APG-1387, or pharmaceutically acceptable salt thereof, and combinations thereof. In some aspects, the human patient is administered a third IAP antagonist selected from the group consisting of ASTX660, or pharmaceutically acceptable salt thereof, SBP-0636457, JP1201, or pharmaceutically acceptable salt thereof, and combinations thereof. The one or more IAP antagonists can be administered simultaneously or at different times during either the day or during the treatment regimen.

In some aspects, the human patient is administered at least one IAP antagonist that is Compound A, or pharmaceutically acceptable salt thereof. In some aspects, the human patient is administered about 100 mg to about 500 mg of Compound A, or pharmaceutically acceptable salt thereof, based on its free base daily. In some aspects, the human patient is administered about 100 mg to about 450 mg, about 100 mg to about 400 mg, about 100 mg to about 350 mg, about 150 mg to about 300 mg, or about 200 mg to about 250 mg of Compound A, or pharmaceutically acceptable salt, based on its free base. In some aspects, the human patient is administered about 100 mg, about 125 mg, about 150 mg, about 1750 mg, about 200 mg, about 225 mg, about 250 mg, about 275 mg, about 300 mg, 325 mg, about 350 mg, about 375 mg, about 400 mg, about 425 mg about 450 mg, about 475 mg, or about 500 mg of Compound A, or pharmaceutically acceptable salt, based on its free base daily. In some aspects, the human patient is administered about 100 mg of Compound A, or pharmaceutically acceptable salt, based on its free base daily. In some aspects, the human patient is administered about 150 mg of Compound A, or pharmaceutically acceptable salt, based on its free base, daily. In some aspects, the human patient is administered about 200 mg of Compound A, or pharmaceutically acceptable salt, based on its free base daily.

In some aspects, the human patient is administered a different amount of at least one IAP antagonist (e.g., Compound A, or pharmaceutically acceptable salt), based on its free base, in the first and second regimens. In some aspects, the human patient is administered a higher dose of at least one IAP antagonist (e.g., Compound A, or pharmaceutically acceptable salt), in the first regimen than in the second regimen. In some aspects, the human patient is administered a higher dose of at least one IAP antagonist (e.g., Compound A, or pharmaceutically acceptable salt), in the second regimen than in the first regimen. In some aspects, the human patient is administered the same dose of at least one IAP antagonist (e.g., Compound A, or pharmaceutically acceptable salt) daily, in both the first and second regimens. In some aspects, the human patient is administered about 100 mg of Compound A, or pharmaceutically acceptable salt, based on its free based daily, in both the first and second regimens. In some aspects, the human patient is administered about 150 mg of Compound A, or pharmaceutically acceptable salt, based on its free base, daily, in both the first and second regimens. In some aspects, the human patient is administered about 200 mg of Compound A, or pharmaceutically acceptable salt, based on its free base daily, in both the first and second regimens.

In some aspects, the platinum-based chemotherapeutic drug is administered intravenously. In some aspects, the platinum-based chemotherapeutic drug is one or more selected from the group consisting of cisplatin, carboplatin, oxaliplatin, and nedaplatin.

In some aspects, the platinum-based chemotherapeutic drug is cisplatin. In some aspects, the cisplatin is administered at a dose between about 10 mg/m² weekly and about 150 mg/m² weekly. In some aspects, the cisplatin is administered at a dose between about 20 mg/m² weekly and about 125 mg/m² weekly. In some aspects, the cisplatin is administered at a dose between about 30 mg/m² weekly and about 100 mg/m² weekly. In some aspects, the cisplatin is administered at a dose between about 40 mg/m² weekly and about 100 mg/m² weekly. In some aspects, the cisplatin is administered at a dose between about 50 mg/m² weekly and about 100 mg/m² weekly. In some aspects, the cisplatin is administered at a dose between about 60 mg/m² weekly and about 100 mg/m² weekly. In some aspects, the cisplatin is administered at a dose between about 70 mg/m² weekly and about 100 mg/m² weekly. In some aspects, the cisplatin is administered at a dose of about 10 mg/m², about 20 mg/m², about 30 mg/m², about 40 mg/m², about 50 mg/m², about 60 mg/m², about 70 mg/m², about 80 mg/m², about 90 mg/m2, or about 100 mg/m² weekly. In some aspects, the cisplatin is administered at a dose of about 100 mg/m².

In some aspects, the cisplatin is administered by slow intravenous infusion. In some aspects, the cisplatin is administered by slow intravenous infusion over at least 45 minutes, at least 60 minutes, at least 90 minutes, or at least 120 minutes.

In some aspects, the cisplatin is administered at a dose of about 100 mg/m² by slow intravenous infusion over at least 90 minutes.

In some aspects, the platinum-based chemotherapeutic drug is carboplatin. In some aspects, the carboplatin is administered at a dose sufficient for the human patient to exhibit an area under the curve of about 1.0 to about 5.5 mg*min/mL. In some aspects, the carboplatin is administered at a dose sufficient for the human patient to exhibit an area under the curve of about 1.0 to about 5.0 mg*min/mL. In some aspects, the carboplatin is administered at a dose sufficient for the human patient to exhibit an area under the curve of about 1.0 to about 4.5 mg*min/mL. In some aspects, the carboplatin is administered at a dose sufficient for the human patient to exhibit an area under the curve of about 1.0 to about 4.0 mg*min/mL. In some aspects, the carboplatin is administered at a dose sufficient for the human patient to exhibit an area under the curve of about 1.0 to about 3.5 mg*min/mL. In some aspects, the carboplatin is administered at a dose sufficient for the human patient to exhibit an area under the curve of about 1.0 to about 3.0 mg*min/mL. In some aspects, the carboplatin is administered at a dose sufficient for the human patient to exhibit an area under the curve of about 1.0 to about 2.5 mg*min/mL. In some aspects, the carboplatin is administered at a dose sufficient for the human patient to exhibit an area under the curve of about 1.0 to about 2.0 mg*min/mL. In some aspects, the carboplatin is administered at a dose sufficient for the human patient to exhibit an area under the curve of about 1.5 to about 2.0 mg*min/mL weekly.

In some aspects, the carboplatin is administered at a dose sufficient for the human patient to exhibit an area under the curve of about 3.0 to about 5.5 every three weeks. In some aspects, the carboplatin is administered at a dose sufficient for the human patient to exhibit an area under the curve of about 3.5 to about 5.5 every three weeks. In some aspects, the carboplatin is administered at a dose sufficient for the human patient to exhibit an area under the curve of about 4.0 to about 5.5 every three weeks. In some aspects, the carboplatin is administered at a dose sufficient for the human patient to exhibit an area under the curve of about 4.5 to about 5.0 every three weeks.

In some aspects, treatment may be initiated with administration of cisplatin and be continued with the administration of carboplatin in replacement of cisplatin.

In some aspects, before each administration of the platinum-based chemotherapeutic drug of the first regimen, the human patient's creatine clearance should be >59 ml/min/1.73 m². In some aspects, before each administration of the platinum-based chemotherapeutic drug of the first regimen, the human patient should not present any neurotoxicity or ototoxicity of grade 2 or above.

In some aspects, the human patient is also administered radiation. In some aspects, the human patient is provided a suitable amount of radiation up to a maximum of 100 grays. In some aspects, the human patient is provided a suitable amount of radiation up to a maximum of 85 grays. In some aspects, the human patient is provided a suitable amount of radiation up to a maximum of 70 grays. In some aspects, the human patient is provided a suitable amount of radiation up to a maximum of 50 grays. In some aspects, the radiation is IMRT.

In some aspects, the human patient is provided a suitable amount of radiation up to a maximum of 70 grays in the first regimen. In some aspects, the human patient is provided a suitable amount of radiation up to a maximum of 50 grays. In some aspects, the radiation is IMRT. In some aspects, radiotherapy should be delivered over seven weeks as follows:
a. To the gross-tumor volume (“GTV”): 5 fractions per week (5/7 days), 1 fraction per day, 2.0 grays/fraction, up to a total of 70 grays;
b. As elective irradiation to locoregional areas: 5 fractions per week (5/7 days), 1 fraction per day, 2.0 grays/fraction up to 50 grays.

In some aspects, if radiation therapy is put on hold for administrative reasons, the total dose planned can be delivered up to week nine of the first regimen.

In some aspects, for a human patient to have radiotherapy (e.g., IMRT), on Day 1 of the second and third cycles of the first regimen, the human patient must meet the following criteria: ≥1000/mm³ (per microliter) ANC (absolute neutrophil count), ≥75000/mm³(per microliter) platelets, Hb ≥8.0 g/dL, albumin ≥1.8 g/dL, ALT/AST ≤×5 ULN, Total bilirubin ≤×2 ULN.

In some aspects, when both radiation therapy and platinum-based chemotherapy are to be administered, the at least one IAP antagonist (e.g., Compound A or a pharmaceutically acceptable salt thereof) can be administered 30 minutes to 3 hours before the platinum-based chemotherapy is administered. In some aspects, when both radiation therapy and platinum-based chemotherapy are to be administered, the platinum-based chemotherapy can be administered before the radiotherapy.

In some aspects, on days when only radiation therapy is to be administered, the at least one IAP antagonist (e.g., Compound A or a pharmaceutically acceptable salt thereof) is to be administered before the radiotherapy.

In some aspects, the at least one IAP antagonist (e.g., Compound A or a pharmaceutically acceptable salt thereof) is to be taken by the human patient in the morning. In some aspects, if the dose administration of the at least one IAP antagonist (e.g., Compound A or a pharmaceutically acceptable salt thereof) is delayed for a reason not related to toxicity, the human patient may take the dose of the IAP antagonist (e.g., Compound A or a pharmaceutically acceptable salt thereof) no later than six hours after normal intake time. If the delay exceeds six hours, the human patient must wait for the next scheduled intake.

In some aspects, if the human patient vomits after administration of at least one IAP antagonist (e.g., Compound A or a pharmaceutically acceptable salt thereof), no additional doses of the IAP antagonist should be re-administered. Rather, the human patient must wait for the next scheduled intake.

In some aspects, if the treatment of the human patient is interrupted for a reason not caused by an adverse reaction related to the at least one IAP antagonist (e.g., Compound A or a pharmaceutically acceptable salt thereof), the treatment can be resumed at the same dose only if the patient did not require more than one treatment interruption per cycle and the interruption lasted a maximum of three consecutive days.

In some aspects, a human patient should miss no more than two doses of radiation therapy (e.g., IMRT) in one week. In some aspects, if two doses of radiation therapy (e.g., IMRT) are missed in one week, a second fraction can be added on a day of the same week. In some aspects, the two fractions of radiation therapy (e.g., IMRT) should be separated by a minimum of six hours.

In some aspects, at least one IAP antagonist (e.g., Compound A, or pharmaceutically acceptable salt thereof), is administered to the human patient in the first regimen after the human patient has fasted for at least two hours. In some aspects, the human patient fasts for at least one hour after at least one IAP antagonist (e.g., Compound A, or pharmaceutically acceptable salt thereof), is administered to the human patient in the first regimen.

In some aspects, at least one IAP antagonist (e.g., Compound A, or pharmaceutically acceptable salt thereof), is administered to the human patient in the second regimen after the human patient has fasted for at least two hours. In some aspects, the human patient fasts for at least one hour after at least one IAP antagonist (e.g., Compound A, or pharmaceutically acceptable salt thereof), is administered to the human patient in the second regimen.

In some aspects, the one or more IAP antagonists (e.g., Compound A, or pharmaceutically acceptable salt thereof), is administered to the human patient in combination with a 5-HT3 receptor antagonist, dexamethasone, and/or aprepitant. In some aspects, the 5-HT3 receptor antagonist is granisetron or palanosetron. In some aspects, the 5-HT3 receptor antagonist is not ondansetron.

In some aspects, the one or more IAP antagonists (e.g., Compound A, or pharmaceutically acceptable salt thereof) is not administered in combination with grapefruit juice, grapefruit-containing products, St. John's Wort (millepertuis), or St. John's Wort-containing products. In some aspects, concomitant intake of inhibitors/inducers of P-gp with one or more IAP antagonists (e.g., Compound A, or pharmaceutically acceptable salt thereof) is prohibited. In some aspects, concomitant intake or administration of CYP3A4 low therapeutic index drugs (drugs metabolized by CYP3A4 for which the range between the effective and toxic concentration is low) or CYP3A4 sensitive substrates (drugs for which CYP3A4 metabolism is highly sensitive to small changes in CY3A4 metabolic capacities) with one or more IAP antagonists (e.g., Compound A, or pharmaceutically acceptable salt thereof) is prohibited. In some aspects, concomitant intake with one or more IAP antagonists (e.g., Compound A, or pharmaceutically acceptable salt thereof) of amiodarone, atorvastatin, carbamazepine, clarithromycin, diltiazem, dronedarone, dipyridamole, erythromycin, fidaxomicin, hydroquinidine, itraconazole, ketoconazole, lansoprazole, mirabegron, propafenone, quinine, quinidine, ranolazine, retigabine, telithromycine, ticagrelor, ulipristal, verapamil, alfuzosin, apixaban, dapoxetine, artemether, atazanavir, atorvastatin, avanafil, bosentan, bromocriptine, ergotamine dihydrate, domperidone, ebastine, eletriptan, eplerenone, ergotamine, fesoterodine, halofantrine, ivabradine, lumefantrine, mizolastine, manidipine, piperaquine, quetiapine, rivaroxaban, rupatadine, sildenafil, simvastatin, solifenacin, tadalafil, tamsulosin, tolterodine, vardenafil, or voriconazole is prohibited.

In some aspects in connection with embodiment A, an IAP antagonist other than compound A, as described hereinabove for embodiment B, is used.

In some aspects in connection with embodiment A or embodiment B, concomitant administration of drugs having a known risk of QTc prolongation is avoided.

In some aspects in connection with embodiment A or embodiment B, concomitant administration of anti-tumor necrosis factor (anti-TNF) therapy (for instance as described in “Anti-TNF therapy: past, present and future” by Claudia Monaco, Jagdeep Nanchahal, Peter Taylor, Marc Feldmann, International Immunology, Volume 27, Issue 1, January 2015, Pages 55-62, https://doi.org/10.1093/intimm/dxu102) with one or more IAP antagonists (e.g., Compound A, or pharmaceutically acceptable salt thereof) is prohibited.

In some aspects in connection with embodiment A or embodiment B, live attenuated vaccination is prohibited during treatment with Debio 1143. In some aspects, live attenuated vaccination is prohibited during the first and second regimens of embodiment A or B. In some aspects, live attenuated vaccination is prohibited within 30 days prior to the first regimen of embodiment A or B. In some aspects, live attenuated vaccination is prohibited within 30 days prior to the first regimen of embodiment A or B and during the first regimen of embodiment A or B. In some aspects, live attenuated vaccination is prohibited within 30 days prior to the first regimen of embodiment A or B and during the first and second regimens of embodiment A or B. In some aspects, life attenuated vaccination is prohibited up to 90 days after the end of treatment.

In some aspects in connection with embodiment A or embodiment B, in particular in the first regimen of embodiment A or B, the cisplatin is administered every three weeks. In some aspects, the cisplatin is administered on Day 2 of each 21-day cycle. In some aspects, the cisplatin is administered at a dose of about 50 mg/m² or more to about 100 mg/m² or less on Day 2 of a 21-day cycle. In some aspects, the cisplatin is administered at a dose of about 50 mg/m² on Day 2 of a 21-day cycle. In some aspects, the cisplatin is administered at a dose of about 75 mg/m² on Day 2 of a 21-day cycle. In some aspects, the cisplatin is administered at a dose of about 100 mg/m² on Day 2 of a 21-day cycle. In some aspects, the cisplatin is initially administered at a dose of about 100 mg/m² on Day 2 of at least the first 21-day cycle and a reduced dose is administered on Day 2 of at least one later 21-day cycle after treatment-related toxicity is observed. In some of these aspects, the dose is reduced to 75 mg/m² or 50 mg/m².

In some aspects in connection with embodiment A or embodiment B, in particular in the first regimen of embodiment A or B, carboplatin is administered every three weeks. In some aspects, the carboplatin is administered on Day 2 of each 21-day cycle. In some aspects, the carboplatin is administered on Day 2 of each 21-day cycle such that a carboplatin AUC of 4 to 5 mg min/mL is reached. In some aspects, cisplatin is initially administered on Day 2 of at least the first 21-day cycle and carboplatin is administered on Day 2 of at least one later 21-day cycle after cisplatin treatment-related toxicity is observed.

In some aspects in connection with the methods described herein, for example embodiment A or embodiment B, cisplatin is initially administered on Day 2 of at least the first 21-day cycle, and if cisplatin related toxicity is observed, either a reduced dose of cisplatin is administered on Day 2 of at least one later 21-day cycle, and/or carboplatin is administered on Day 2 of at least one later 21-day cycle. In some of these aspects, the cisplatin dose is reduced to 75 mg/m² or 50 mg/m². In some aspects, carboplatin is administered so that an AUC of 4 to 5 mg min/mL is reached.

In some aspects in connection with embodiment A or embodiment B, the platinum-based chemotherapeutic drug, e.g., cisplatin or carboplatin, is administered by slow intravenous infusion over at least 45 minutes, at least 60 minutes, at least 90 minutes, or at least 120 minutes.

In some aspects in connection with embodiment A or embodiment B, in particular in the first regimen of embodiment A or B, cisplatin is administered by intravenous infusion over at least 90 minutes. In some aspects, the cisplatin is administered every three weeks on Day 2 of each 21-day cycle by intravenous infusion over at least 90 minutes. In some aspects, the cisplatin is administered at a dose of about 100 mg/m² every three weeks on Day 2 of each 21-day cycle by intravenous infusion over at least 90 minutes.

In some aspects in connection with embodiment A or embodiment B, in particular in the first regimen of embodiment A or B, the carboplatin is administered by intravenous infusion over at least 90 minutes. In some aspects, the carboplatin is administered every three weeks on Day 2 of each 21-day cycle by intravenous infusion over at least 90 minutes. In some aspects, the carboplatin is administered at a dose such that a carboplatin AUC of 4 to 5 mg min/mL is reached every three weeks on Day 2 of each 21-day cycle by intravenous infusion over at least 90 minutes.

In some aspects in connection with embodiment A or embodiment B, in particular in the first regimen of embodiment A or B, the human patient is provided with radiation to the gross tumor volume of a total amount up to a maximum of 70 grays. In some aspects, the radiation to the gross tumor volume is provided in several fractions with 1 fraction per day of 2.0 grays per fraction. In some aspects, the radiation to the gross tumor volume is provided in several fractions with 1 fraction per day of 2.0 grays per fraction in 5 days/7 days up to a total of 35 fractions. In some of these aspects, radiation is IMRT.

In some aspects in connection with embodiment A or embodiment B, in particular in the first regimen of embodiment A or B, the human patient is provided with radiation to the gross tumor volume (e.g. primary tumor) of a total amount up to 70 grays, and a reduced radiation dose (elective radiation) of a total amount up to a maximum of 56 grays to locoregional areas (low-risk subclinical sites such as nodes at the periphery of the primary tumor). In some aspects, the elective radiation to locoregional areas is provided in several fractions with 1 fraction per day of 1.6 grays per fraction. In some aspects, the elective radiation to locoregional areas is provided in several fractions with 1 fraction per day of 1.6 grays per fraction in 5 days/7 days up to a total of 35 fractions. In some aspects, the elective radiation to locoregional areas is provided in several fractions with 1 fraction per day of 1.6 grays per fraction and the radiation to the gross tumor volume is provided in several fractions with 1 fraction per day of 2.0 grays per fraction. In some aspects, the elective radiation to locoregional areas is provided in several fractions with 1 fraction per day of 1.6 grays per fraction in 5 days/7 days up to a total of 35 fractions and the radiation to the gross tumor volume is provided in several fractions with 1 fraction per day of 2.0 grays per fraction in 5 days/7 days up to a total of 35 fractions. In some of these aspects, radiation is IMRT.

In some aspects in connection with embodiment A or embodiment B, in particular in the first regimen of embodiment A or B, radiotherapy is delivered over seven weeks as follows:
a. To the gross-tumor volume (“GTV”): 5 fractions per week (5/7 days), 1 fraction per day, 2.0 grays/fraction, up to a total of 70 grays;
b. As elective irradiation to locoregional areas: 5 fractions per week (5/7 days), 1 fraction per day, 1.6 grays/fraction up to 56 grays.

In some aspects in connection with embodiment A or embodiment B, at least one IAP antagonist (e.g., Compound A, or pharmaceutically acceptable salt thereof) is administered to the human patient after the human patient has fasted for at least one hour. In some aspects, the at least one IAP antagonist (e.g., Compound A, or pharmaceutically acceptable salt thereof) is administered to the human patient after fasting overnight, i.e. after waking up and before breakfast. This is typically done in the early morning, e.g., between 6 am and 9 am.

In some aspects in connection with embodiment A or embodiment B, the human patient fasts for at least one hour after at least one IAP antagonist (e.g., Compound A, or pharmaceutically acceptable salt thereof) is administered to the human patient.

In some aspects in connection with embodiment A or embodiment B, in particular in the first regimen of embodiment A or B, on days when radiation and a platinum-based chemotherapeutic drug are to be administered, the at least one IAP antagonist (e.g., Compound A or pharmaceutically acceptable salt thereof) is administered between 3 hours and 30 minutes before the platinum-based chemotherapeutic drug. In some aspects, on days when radiation and a platinum-based chemotherapeutic drug are to be administered, the platinum-based chemotherapeutic drug is administered before radiation, e.g., IMRT. In some aspects, on days when radiation and a platinum-based chemotherapeutic drug are to be administered, the at least one IAP antagonist, e.g., Compound A or pharmaceutically acceptable salt thereof, is administered between 3 hours and 30 minutes before the platinum-based chemotherapeutic drug, e.g., cisplatin or carboplatin, and the platinum-based chemotherapeutic drug is administered before radiation, e.g., IMRT.

In some aspects in connection with embodiment A or embodiment B, in particular in the first regimen of embodiment A or B, on days when IMRT and cisplatin or carboplatin are to be administered, Compound A or pharmaceutically acceptable salt thereof is administered between 3 hours and 30 minutes before the cisplatin or carboplatin. In some aspects, on days when IMRT and a platinum-based chemotherapeutic drug are to be administered, the cisplatin or carboplatin is administered before IMRT. In some aspects, on days when IMRT and cisplatin or carboplatin are to be administered, Compound A or pharmaceutically acceptable salt thereof is administered between 3 hours and 30 minutes before the cisplatin or carboplatin, and the cisplatin or carboplatin is administered before IMRT.

In some aspects in connection with embodiment A or embodiment B, in particular in the first regimen of embodiment A or B, on days when radiation is to be administered without a platinum-based chemotherapeutic drug, the at least one IAP antagonist, e.g., Compound A or pharmaceutically acceptable salt thereof, is administered before radiation, e.g., IMRT.

In some aspects in connection with embodiment A or embodiment B, in particular in the first regimen of embodiment A or B, on days when IMRT is to be administered without a platinum-based chemotherapeutic drug, Compound A or pharmaceutically acceptable salt thereof is administered before IMRT.

In some aspects in connection with embodiment A or embodiment B, the second regimen comprises three consecutive 21-day cycles.

In some aspects in connection with embodiment A or embodiment B, at least one IAP antagonist (e.g., Compound A, or pharmaceutically acceptable salt thereof), is administered to the human patient in the first regimen after the human patient has fasted for at least one hour. In some aspects in connection with embodiment A or embodiment B, the human patient fasts for at least one hour after at least one IAP antagonist (e.g., Compound A, or pharmaceutically acceptable salt thereof), is administered to the human patient in the first regimen. In some aspects in connection with embodiment A or embodiment B, in the first regimen, the human patient fasts for at least one hour before Compound A or pharmaceutically acceptable salt thereof is administered and at least one hour after Compound A or pharmaceutically acceptable salt thereof is administered to the human patient.

In some aspects in connection with embodiment A or embodiment B, at least one IAP antagonist (e.g., Compound A, or pharmaceutically acceptable salt thereof), is administered to the human patient in the second regimen after the human patient has fasted for at least one hour. In some aspects in connection with embodiment A or embodiment B, the human patient fasts for at least one hour after at least one IAP antagonist (e.g., Compound A, or pharmaceutically acceptable salt thereof), is administered to the human patient in the second regimen. In some aspects in connection with embodiment A or embodiment B, in the second regimen, the human patient fasts for at least one hour before Compound A or pharmaceutically acceptable salt thereof is administered and at least one hour after Compound A or pharmaceutically acceptable salt thereof is administered to the human patient.

In some aspects in connection with embodiment A or embodiment B, the features of one or more of the appended claims may be combined with one or more features disclosed in the present description hereinabove and/or hereinbelow. Likewise, any one of the following aspects (a) to (q) may be combined with one or more features disclosed in the present description hereinabove and/or hereinbelow:
(a) A method for treating a human patient having locally advanced squamous cell carcinoma, wherein the method comprises administering to the patient Compound A, or pharmaceutically acceptable salt thereof, cisplatin or carboplatin, and IMRT for at least one cycle of 21 days.
(b) The method of aspect (a), wherein Compound A, or pharmaceutically acceptable salt thereof, is administered from Day 1 to Day 14 of the at least one cycle of 21 days at a dose corresponding to 100 mg per day or more to 200 mg per day or less of Compound A as its free base.
(c) The method of aspect (b), wherein Compound A, or pharmaceutically acceptable salt thereof, is administered at a dose corresponding to 200 mg per day of Compound A as its free base.
(d) The method of aspect (b) or (c), wherein Compound A, or pharmaceutically acceptable salt thereof or is initially administered at least once at a dose corresponding to 200 mg per day of Compound A as its free base and wherein subsequently Compound A, or pharmaceutically acceptable salt thereof, is administered at least once at a reduced dose corresponding to 150 mg per day or 100 mg per day of Compound A as its free base.
(e) The method of aspect (b), (c) or (d), wherein Compound A, or pharmaceutically acceptable salt thereof, is administered after at least 1 hour of fasting.
(f) The method of aspect (e), wherein Compound A, or pharmaceutically acceptable salt thereof, is administered followed by at least 1 hour of fasting.
(g) The method of aspect (f), wherein cisplatin is administered at Day 2 of the at least one cycle of 21 days at a dose of 50 mg/m² per day or more to 100 mg/m² per day or less.
(h) The method of aspect (g), wherein cisplatin is administered at a dose of 100 mg/m² per day.
(i) The method of aspect (g) or (h), wherein cisplatin is initially administered at least once at a dose of 100 mg/m² per day and wherein subsequently cisplatin is administered at least once at a reduced dose of 50 mg/m² per day or 75 mg/m² per day.
(j) The method of aspect (g), (h) or (i), wherein cisplatin is administered by intravenous infusion over a time period of at least 90 min.
(k) The method of aspect (j), wherein IMRT is administered to the gross tumor volume in fractions with 1 fraction on each of 5 days per 7 days, i.e. on Days 1-5, 8-12, and 15-19 of the 21 day cycle, each fraction having radiation of 2.0 gray.
(l) The method of aspect (k), wherein elective irradiation is administered to locoregional areas in fractions with 1 fraction on each of 5 days per 7 days, i.e. on Days 1-5, 8-12, and 15-19 of the 21 day cycle, each fraction having radiation of 1.6 gray.
(m) The method of aspect (l), wherein on days when compound A or a pharmaceutically acceptable salt thereof, cisplatin or carboplatin and IMRT are administered, compound A or a pharmaceutically acceptable salt thereof is administered first, followed by administration of cisplatin or carboplatin followed by administration of IMRT.
(n) The method of any of aspects (a) to (m), wherein cisplatin is administered at least during the first cycle and wherein, if cisplatin-related toxicity or adverse effects are encountered, the dose of cisplatin may be reduced, for instance by 25% from the prior dose, or cisplatin may be switched to carboplatin. If cisplatin-related toxicity or adverse events remain after reduction of the cisplatin dose, the dose of cisplatin may be further reduced or cisplatin may be switched to carboplatin.
(o) The method of aspect (m) or (n), wherein Compound A, or pharmaceutically acceptable salt thereof, cisplatin or carboplatin, and IMRT are administered for three cycles of 21 days of such combination therapy.
(p) The method of aspect (o), wherein, after the end of the three cycles of combination therapy, the compound A is administered as a monotherapy without administration of any of cisplatin, carboplatin and IMRT for at least one cycle of 21 days, wherein compound A is administered from Day 1 to Day 14 of the at least one cycle of 21 days.
(q) The method of aspect (m), (n), (o) or (p), wherein the patient fulfils one or more of the following criteria:
   (i) the human patient has a positive smoking history, especially of 10 pack-years or more;
   (ii) the human patient is a heavy consumer of alcohol consuming on average 21 drinks per week or more;
   (iii) the human patient has no history of infection with human immunodeficiency virus (HIV);
   (iv) the human patient has no other infections such as viral and/or bacterial and/or mycotic infections requiring a systemic treatment;
   (v) the human patient has no active uncontrolled inflammatory disease, such as rheumatoid arthritis, systemic lupus erythematosus, Sjögren syndrome or severe extensive psoriasis, requiring ongoing treatment with anti-tumor necrosis factor (TNF) medication
   (vi) the human patient has no impaired cardiovascular function or clinically significant cardiovascular diseases;
   (vii) the human patient has no symptomatic pulmonary disease requiring continuous or intermittent oxygen supply;
   (viii) the human patient has no non-compensated or symptomatic liver cirrhosis which shows a Child-Pugh score of B or C;
   (ix) the human patient suffers from a locally advanced squamous cell carcinoma of the head and neck that is stage III, IVA or IVB as per American Joint Committee on Cancer (“AJCC”) TNM staging version 7.0 (2010) or 8.0 (2018). The TNM Staging System is based on the extent of the tumor (T), the extent of spread to the lymph nodes (N), and the presence of metastasis (M);
   (x) the human patient or the LA-SCCHN harbors one or more mutations of the p53 gene.

For instance, the treatment specified under items (a) to (o) and (q) in the previous paragraph may be incorporated as a first treatment regimen in the treatments including a first and second regimen as described hereinabove.

In some aspects in connection with embodiment A or embodiment B, the treatment with IAP antagonist and especially compound A may be used to sensitize tumor cells to CRT by promoting apoptosis (programmed cell death) and fostering antitumor immunity.

In some aspects in connection with embodiment A or embodiment B, the treatment with IAP antagonist and especially compound A may be used to optimize the effectiveness of chemoradiotherapy (CRT) when administered together.

Also provided herein is a method for treating a human patient having locally advanced squamous cell carcinoma comprising administering to the human patient in need thereof, an inhibitor of apoptosis protein (“IAP”) antagonist (e.g. Compound A or a pharmaceutically acceptable salt thereof), wherein the method consists of a regimen as described above for the first regimen of any of the aspects of either of embodiments A and B.

In any aspects of the methods described herein, for example in connection with embodiment A or embodiment B, when a particular toxicity or adverse event is encountered, any treatment modification corresponding to such toxicity or adverse event and listed as an “action” in Table 4 or alternatively in Tables 5 or 6, may be combined with any of the features of such methods. In any aspects of the methods described herein, for example in connection with embodiment A or embodiment B, when two or more particular toxicities or adverse events are encountered, any respective treatment modifications corresponding to such toxicities or adverse events and listed as “actions” in Table 4 or alternatively in Tables 5 or 6, may be combined with any of the features of such methods.

### III. Patients

The patients that can be treated according to embodiment A are described in the following.

In some aspects of the above methods, the human patient has a positive smoking history. In some aspects of the above methods, the human patient is a heavy consumer of alcohol.

In some aspects of the above methods, the human patient has not previously received treatment (e.g., surgery, chemotherapy, radiotherapy, immunotherapy) of the locally advanced squamous cell carcinoma. In some aspects of the above methods, the human patient has previously received treatment (e.g., surgery, chemotherapy, radiotherapy, immunotherapy) for a cancer other than a locally advanced squamous cell carcinoma (e.g., breast cancer, lymphoma). In some aspects of the above methods, the human patient has previously had surgical treatment for locally advanced squamous cell carcinoma, but not received another type of treatment (e.g., chemotherapy, radiotherapy, immunotherapy). In some aspects of the above methods, the human patient has previously received treatment (e.g., surgery, chemotherapy, radiotherapy, immunotherapy) for locally advanced squamous cell carcinoma located other than on the head or neck.

In some aspects of the above methods, the locally advanced squamous cell carcinoma is inoperable. In some aspects of the above methods, the locally advanced squamous cell carcinoma is Stage III, Stage IVA, or Stage IVB according to the American Joint Committee on Cancer 1998 staging system.

In some aspects of the above methods, the patient has a locally advanced squamous cell carcinoma of the head and neck region. In some aspects of the above methods, the locally advanced squamous cell carcinoma of the head and neck region is inoperable. In some aspects of the above methods, the locally advanced squamous cell carcinoma of the head and neck region is selected from the group consisting of oral cavity, oropharynx, larynx, and hypopharynx. In some aspects of the above methods, the locally advanced squamous cell carcinoma of the head and neck region is oropharyngeal cancer. In some aspects of the above methods, the oropharyngeal cancer is unrelated to human papillomavirus (HPV) infection (as determined by p16 immunohistochemistry (IHC)). In some aspects of the above methods, the oropharyngeal cancer is related to human papillomavirus (HPV) infection (as determined by p16 immunohistochemistry (IHC)). In some aspects of the above methods, the locally advanced squamous cell carcinoma of the head or neck is Stage III, Stage IVA, or Stage IVB according to the American Joint Committee on Cancer 1998 staging system.

The patients that can be treated according to embodiment B are described in the following.

In some aspects of the above methods, the human patient has a positive smoking history. In some aspects of the above methods, the human patient is a heavy consumer of alcohol. In some aspects of the above methods, the human patient has HPV-negative OPC. In some aspects of the above methods, the human patient has a positive smoking history, is a heavy consumer of alcohol, and/or has HPV-negative OPC.

In some aspects of the above methods, the human patient has not previously received treatment (e.g., surgery, chemotherapy, radiotherapy, immunotherapy) of the locally advanced squamous cell carcinoma. In some aspects of the above methods, the human patient has previously received treatment (e.g., surgery, chemotherapy, radiotherapy, immunotherapy) for a cancer other than a locally advanced squamous cell carcinoma (e.g., breast cancer, lymphoma). In some aspects of the above methods, the human patient has previously had surgical treatment for locally advanced squamous cell carcinoma, but not received another type of treatment (e.g., chemotherapy, radiotherapy, immunotherapy). In some aspects of the above methods, the human patient has previously received treatment (e.g., surgery, chemotherapy, radiotherapy, immunotherapy) for locally advanced squamous cell carcinoma located other than on the head or neck.

In some aspects of the above methods, the locally advanced squamous cell carcinoma is inoperable. In some aspects of the above methods, the locally advanced squamous cell carcinoma is Stage III, Stage IVA, or Stage IVB according to the American Joint Committee on Cancer 1998 staging system.

In some aspects of the above methods, the human patient has a locally advanced squamous cell carcinoma of the head and neck region. In some aspects of the above methods, the locally advanced squamous cell carcinoma of the head and neck region is inoperable. In some aspects of the above methods, the locally advanced squamous cell carcinoma of the head and neck region is selected from the group consisting of, oropharynx, larynx, and hypopharynx. In some aspects of the above methods, the locally advanced squamous cell carcinoma of the head and neck region is oropharyngeal cancer. In some aspects of the above methods, the oropharyngeal cancer is unrelated to human papillomavirus (HPV) infection (as determined by p16 immunohistochemistry (IHC)). In some aspects of the above methods, the oropharyngeal cancer is related to human papillomavirus (HPV) infection (as determined by p16 immunohistochemistry (IHC)). In some aspects of the above methods, the locally advanced squamous cell carcinoma of the head or neck is Stage III, Stage IVA, or Stage IVB according to the American Joint Committee on Cancer 1998 staging system.

In some aspects in connection with embodiment A or embodiment B, the human patient has a positive smoking history of 10 pack-years. In some aspects, the human patient is a heavy consumer of alcohol, consuming in average 21 drinks per week. In some aspects, the human patient has a positive smoking history of 10 pack-years and/or is a heavy consumer of alcohol consuming in average 21 drinks per week.

In some aspects in connection with embodiment A or embodiment B, the human patient has no history of infection with human immunodeficiency virus (HIV).

In some aspects in connection with embodiment A or embodiment B, the human patient has no other infections such as viral and/or bacterial and/or mycotic infection requiring systemic treatment.

In some aspects in connection with embodiment A or embodiment B, the human patient has no active uncontrolled inflammatory disease, such as rheumatoid arthritis, systemic lupus erythematosus, Sjögren syndrome or severe extensive psoriasis, requiring ongoing treatment with anti-tumor necrosis factor (TNF) medication.

In some aspects in connection with embodiment A or embodiment B, the human patient has no impaired cardiovascular function or clinically significant cardiovascular diseases, including any of the following:
a. Ongoing or history of uncontrolled or symptomatic ischemic myocardiopathy
b. Known left ventricular ejection fraction <50%, left ventricular hypertrophy, ventricular arrhythmias, bradycardia (heart rate <50 bpm)
c. History of myocardial infarction, or severe/unstable angina
d. New York Heart Association grade ≥3 congestive heart failure
e. Congenital long QT syndrome
f. Family history of long QT syndrome
g. Symptomatic pulmonary embolism
h. Ongoing or known history of transient ischemic attacks or stroke
i. QTc using Fridericia's formula (QTcF) interval >450 ms for males and >470 ms for females.

In some aspects in connection with embodiment A or embodiment B, the human patient has no symptomatic pulmonary disease requiring continuous or intermittent oxygen supply.

In some aspects in connection with embodiment A or embodiment B, the human patient has no non-compensated or symptomatic liver cirrhosis of Child-Pugh score B or C. In some aspects, the human patient has no non-compensated or symptomatic liver cirrhosis of Child-Pugh score B.

In some aspects in connection with the methods described herein, for example in connection with embodiment A or B, the human patient patient suffers from a locally advanced squamous cell carcinoma of the head and neck that is stage III, IVA or IVB as per American Joint Committee on Cancer (“AJCC”) TNM staging version 7.0 (2010) or 8.0 (2018). The TNM Staging System is based on the extent of the tumor (T), the extent of spread to the lymph nodes (N), and the presence of metastasis (M).

In some aspects in connection with embodiment A or B, the human patient or the LA-SCCHN harbors one or more mutations of the p53 gene.

### IV. Pharmaceutical Compositions

The pharmaceutical compositions of embodiment A are described in the following.

In some aspects of the above methods, Compound A is in free base form. In some aspects of the above methods, Compound A is a pharmaceutically acceptable salt. In some aspects of the above methods, Compound A is a pharmaceutically acceptable salt form selected from the group consisting of acetate, hydrochloride, citrate, lactate, fumarate, succinate, phosphate, maleate, sulfate, tartrate, benzoate, mesylate, malate, hydrobromide, tosylate, nitrate, N-acetylglycine, ascorbate, butanate, ethane-1,2-disulfonate, gentisate, glucoronate, glutarate, glycolate, isethionate, ketoglutarate, malonate, napadisylate, napsylate, nicotinate, pyroglutamate, pyruvate, sebacate, and succinate. In some aspects of the above methods, the pharmaceutically acceptable form is selected from the group consisting of sulfate, tosylate, gentisate, hydrochloride, napadisylate, napsylate, laurylsulfate, xinafoate, and pamoate.

In some aspects of the above methods, Compound A, or pharmaceutically acceptable salt thereof, is administered orally. In some aspects of the above methods, Compound A, or pharmaceutically acceptable salt thereof, is administered as a solid dosage form. In some aspects of the above methods, the solid dosage form is a capsule or tablet.

In some aspects of the above methods, the solid dosage form comprises an amount of Compound A, or pharmaceutically acceptable salt thereof, corresponding to about 20 mg, about 25 mg, about 30 mg, about 40 mg, about 50 mg, about 60 mg, about 70 mg, about 75 mg, about 80 mg, about 90 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, or about 500 mg of Compound A as its free base. The amount of Compound A, as a pharmaceutically acceptable salt, will be adjusted based on the weight of the salt form to be included in the solid dosage form.

In some aspects of the above methods, Compound A, or pharmaceutically acceptable salt thereof, is administered as a solution. In some aspects of the above methods, Compound A, or pharmaceutically acceptable salt thereof, is administered as an aqueous solution. In some aspects of the above methods, Compound A, or pharmaceutically acceptable salt thereof, is administered orally via a nasogastric tube, percutaneous endoscopic gastrostomy tube, or percutaneous endoscopic jejunostomy tube.

In some aspects of the above methods, the solution (e.g., aqueous solution) comprises Compound A, or pharmaceutically acceptable salt thereof, at a concentration of between about 10 mg/mL to about 50 mg/mL, about 10 mg/mL to about 40 mg/mL, or about 10 mg/mL to about 30 mg/mL based on Compound A as its free base. In some aspects of the above methods, the solution (e.g., aqueous solution) comprises Compound A, or pharmaceutically acceptable salt thereof, at a concentration of about 10 mg/mL, about 20 mg/mL, about 25 mg/mL, about 30 mg/mL, about 40 mg/mL, or about 50 mg/mL based on Compound A, as its free base. In some aspects of the above methods, the solution (e.g., aqueous solution) comprises Compound A, or pharmaceutically acceptable salt thereof, at a concentration of about 20 mg/mL based on Compound A, as its free base.

In some aspects of the above methods, Compound A, or pharmaceutically acceptable salt thereof, is administered as one dose one time per day. In some aspects of the above methods, Compound A, or pharmaceutically acceptable salt thereof, is divided into multiple doses that are administered one, two, three, or four times per day.

The pharmaceutical compositions of embodiment B are described in the following.

In some aspects of the above methods, Compound A is in free base form. In some aspects of the above methods, Compound A is a pharmaceutically acceptable salt. In some aspects of the above methods, Compound A is a pharmaceutically acceptable salt form selected from the group consisting of acetate, hydrochloride, citrate, lactate, fumarate, succinate, phosphate, maleate, sulfate, tartrate, benzoate, mesylate, malate, hydrobromide, tosylate, nitrate, N-acetylglycine, ascorbate, butanate, ethane-1,2-disulfonate, gentisate, glucoronate, glutarate, glycolate, isethionate, ketoglutarate, malonate, napadisylate, napsylate, nicotinate, pyroglutamate, pyruvate, sebacate, and succinate. In some aspects of the above methods, the pharmaceutically acceptable form is selected from the group consisting of sulfate, tosylate, gentisate, hydrochloride, napadisylate, napsylate, laurylsulfate, xinafoate, and pamoate.

In some aspects of the above methods, Compound A, or pharmaceutically acceptable salt thereof, is administered orally. In some aspects of the above methods, Compound A, or pharmaceutically acceptable salt thereof, is administered as a solid dosage form. In some aspects of the above methods, the solid dosage form is a capsule or tablet.

In some aspects of the above methods, the solid dosage form comprises an amount of Compound A, or pharmaceutically acceptable salt thereof, corresponding to about 20 mg, about 25 mg, about 30 mg, about 40 mg, about 50 mg, about 60 mg, about 70 mg, about 75 mg, about 80 mg, about 90 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, or about 500 mg of Compound A as its free base. The amount of Compound A, as a pharmaceutically acceptable salt, will be adjusted based on the weight of the salt form to be included in the solid dosage form.

In some aspects of the above methods, Compound A, or pharmaceutically acceptable salt thereof, is administered as a solution. In some aspects of the above methods, Compound A, or pharmaceutically acceptable salt thereof, is administered as an aqueous solution. In some aspects of the above methods, Compound A, or pharmaceutically acceptable salt thereof, is administered orally via a nasogastric tube, percutaneous endoscopic gastrostomy tube, or percutaneous endoscopic jejunostomy tube.

In some aspects of the above methods, the solution (e.g., aqueous solution) comprises Compound A, or pharmaceutically acceptable salt thereof, at a concentration of between about 10 mg/mL to about 50 mg/mL, about 10 mg/mL to about 40 mg/mL, or about 10 mg/mL to about 30 mg/mL based on Compound A as its free base. In some aspects of the above methods, the solution (e.g., aqueous solution) comprises Compound A, or pharmaceutically acceptable salt thereof, at a concentration of about 10 mg/mL, about 20 mg/mL, about 25 mg/mL, about 30 mg/mL, about 40 mg/mL, or about 50 mg/mL based on Compound A, as its free base. In some aspects of the above methods, the solution (e.g., aqueous solution) comprises Compound A, or pharmaceutically acceptable salt thereof, at a concentration of about 20 mg/mL based on Compound A, as its free base.

In some aspects of the above methods, Compound A, or pharmaceutically acceptable salt thereof, is administered as one dose one time per day. In some aspects of the above methods, Compound A, or pharmaceutically acceptable salt thereof, is divided into multiple doses that are administered one, two, three, or four times per day.

### V. Pharmaceutical Use and Kits

In some aspects of the invention, provided herein is use of an inhibitor of apoptosis protein (“IAP”) antagonist (e.g. Compound A or a pharmaceutically acceptable salt thereof) in manufacture of a medicament for treating locally advanced squamous cell carcinoma via any of the methods as described in the present invention (e.g. those as described in embodiments A and B).

In some aspects, the medicament is a solid dosage form. In some aspects, the solid dosage form is a capsule or tablet. In some aspects, the solid dosage form comprises an amount of Compound A or a pharmaceutically acceptable salt thereof corresponding to about 20 mg, about 25 mg, about 30 mg, about 40 mg, about 50 mg, about 60 mg, about 70 mg, about 75 mg, about 80 mg, about 90 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg or about 500 mg of Compound A as its free base. The amount of Compound A, as a pharmaceutically acceptable salt, will be adjusted based on the weight of the salt form to be included in the solid dosage form.

In some aspects, the medicament is a solution or can be formulated into a solution before use. In some aspects, the solution is an aqueous solution. In some aspects, the solution is administered orally via a nasogastric tube, percutaneous endoscopic gastrostomy tube or percutaneous endoscopic jejunostomy tube. In some aspects, the solution comprises Compound A or pharmaceutically acceptable salt thereof at a concentration of between about 10 mg/mL to about 50 mg/mL, about 10 mg/mL to about 40 mg/mL or about 10 mg/mL to about 30 mg/mL based on Compound A as its free base. In some aspects, the solution comprises Compound A or pharmaceutically acceptable salt thereof at a concentration of about 10 mg/mL, about 20 mg/mL, about 25 mg/mL, about 30 mg/mL, about 40 mg/mL or about 50 mg/mL based on Compound A as its free base. In some aspects, the solution comprises Compound A or pharmaceutically acceptable salt thereof at a concentration of about 20 mg/mL based on Compound A as its free base.

Also provided herein is a kit comprising:
(a) a first pharmaceutical composition comprising an inhibitor of apoptosis protein (“IAP”) antagonist (e.g. Compound A or a pharmaceutically acceptable salt thereof); and
(b) an insert instructing use of the first pharmaceutical composition for treating locally advanced squamous cell carcinoma via any of the methods as described in the present invention (e.g. those as described in embodiments A and B).

In some aspects, the first pharmaceutical composition is a solid dosage form. In some aspects, the solid dosage form is a capsule or tablet. In some aspects, the solid dosage form comprises an amount of Compound A or a pharmaceutically acceptable salt thereof corresponding to about 20 mg, about 25 mg, about 30 mg, about 40 mg, about 50 mg, about 60 mg, about 70 mg, about 75 mg, about 80 mg, about 90 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg or about 500 mg of Compound A as its free base. The amount of Compound A, as a pharmaceutically acceptable salt, will be adjusted based on the weight of the salt form to be included in the solid dosage form.

In some aspects, the first pharmaceutical composition is a solution or can be formulated into a solution before use. In some aspects, the solution is an aqueous solution. In some aspects, the solution is administered orally via a nasogastric tube, percutaneous endoscopic gastrostomy tube or percutaneous endoscopic jejunostomy tube. In some aspects, the solution comprises Compound A or pharmaceutically acceptable salt thereof at a concentration of between about 10 mg/mL to about 50 mg/mL, about 10 mg/mL to about 40 mg/mL or about 10 mg/mL to about 30 mg/mL based on Compound A as its free base. In some aspects, the solution comprises Compound A or pharmaceutically acceptable salt thereof at a concentration of about 10 mg/mL, about 20 mg/mL, about 25 mg/mL, about 30 mg/mL, about 40 mg/mL or about 50 mg/mL based on Compound A as its free base. In some aspects, the solution comprises Compound A or pharmaceutically acceptable salt thereof at a concentration of about 20 mg/mL based on Compound A as its free base.

In some aspects, the kit further comprises: (c) a second pharmaceutical composition comprising a platinum-based chemotherapeutic drug as described in the present invention (e.g. those as described in embodiments A and B). In some aspects, the insert further instructs use of the second pharmaceutical composition for treating locally advanced squamous cell carcinoma via any of the methods as described in the present invention (e.g. those as described in embodiments A and B).

Also provided herein is use of an inhibitor of apoptosis protein (“IAP”) antagonist (e.g. Compound A or a pharmaceutically acceptable salt thereof) in manufacture of a medicament for treating locally advanced squamous cell carcinoma, wherein the IAP antagonist is administered in combination with a platinum-based chemotherapeutic drug. Also provided herein is use of an IAP antagonist (e.g. Compound A or a pharmaceutically acceptable salt thereof) together with a platinum-based chemotherapeutic drug in manufacture of a kit for treating locally advanced squamous cell carcinoma, the kit comprising items (a), (b) and (c) as described above. Also provided herein is a combination of an IAP antagonist (e.g. Compound A or a pharmaceutically acceptable salt thereof) with a platinum-based chemotherapeutic drug for treating locally advanced squamous cell carcinoma.

In some aspects, the IAP antagonist (e.g. Compound A or a pharmaceutically acceptable salt thereof) is administered as described above for it in the first regimen of any of the aspects of either of embodiments A and B.

In some aspects, the platinum-based chemotherapeutic drug is selected from a group consisting of those platinum-based chemotherapeutic drugs as described in embodiments A and B. In some aspects, the platinum-based chemotherapeutic drug is cisplatin. In some aspects, the platinum-based chemotherapeutic drug is carboplatin. In some aspects, the platinum-based chemotherapeutic drug is administered as described above for it in the first regimen of any of the aspects of either of embodiments A and B.

In some aspects, the combination further comprises radiation. In some aspects, the radiation is intensity-modulated radiotherapy (“IMRT”). In some aspects, the radiation is administered as described above for it in the first regimen of any of the aspects of either of embodiments A and B.

In some aspects, the medicament of Compound A is a solid dosage form. In some aspects, the solid dosage form is a capsule or tablet. In some aspects, the solid dosage form comprises an amount of Compound A or a pharmaceutically acceptable salt thereof corresponding to about 20 mg, about 25 mg, about 30 mg, about 40 mg, about 50 mg, about 60 mg, about 70 mg, about 75 mg, about 80 mg, about 90 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg or about 500 mg of Compound A as its free base. The amount of Compound A, as a pharmaceutically acceptable salt, will be adjusted based on the weight of the salt form to be included in the solid dosage form. In some aspects, the medicament of Compound A is a solution or can be formulated into a solution before use. In some aspects, the solution is an aqueous solution. In some aspects, the solution is administered orally via a nasogastric tube, percutaneous endoscopic gastrostomy tube or percutaneous endoscopic jejunostomy tube. In some aspects, the solution comprises Compound A or pharmaceutically acceptable salt thereof at a concentration of between about 10 mg/mL to about 50 mg/mL, about 10 mg/mL to about 40 mg/mL or about 10 mg/mL to about 30 mg/mL based on Compound A as its free base. In some aspects, the solution comprises Compound A or pharmaceutically acceptable salt thereof at a concentration of about 10 mg/mL, about 20 mg/mL, about 25 mg/mL, about 30 mg/mL, about 40 mg/mL or about 50 mg/mL based on Compound A as its free base. In some aspects, the solution comprises Compound A or pharmaceutically acceptable salt thereof at a concentration of about 20 mg/mL based on Compound A as its free base.

### EXAMPLES

It is understood that the examples and aspects described herein are for illustrative purposes only and that various modifications or changes in light thereof will be suggested to persons skilled in the art and are to be included within the spirit and purview of this application.

### Example 1

The maximum tolerated dose (MTD) of Debio 1143 in combination with concurrent chemoradiotherapy (CRT) in subjects with locally advanced squamous cell cancer of the head and neck (LA-SCCHN) was determined.

Additionally, for each Debio 1143 dose level (DL) and overall, according to variant 1 of this example:
(a) the safety profile of Debio 1143 in combination with concurrent CRT was assessed;
(b) the antitumour activity of the recommended dose (RD) of Debio 1143 in combination with concurrent CRT in the study population was evaluated.
In a variant 2 of this example, the following items (c) to (f) were explored in addition to items (a) and (b) of variant 1:
(c) the pharmacokinetics (PK) of Debio 1143 and cisplatin when administered in combination with radiation therapy, and explore the potential for drug-drug interactions of Debio 1143 in combination with cisplatin was explored;
(d) the pharmacodynamic (PDy) biomarkers of Debio 1143 activity in combination with concurrent CRT were explored;
(e) the PK/PDy, PK/efficacy, PK/safety, PDy/efficacy and PDy/safety relationships and pharmacogenetic (PGx) factors that may affect Debio 1143 PK were explored; and
(f) the pharmacogenomic and tumour PGx factors were explored.

During the screening phase, subjects were evaluated and staged using the American Joint Committee on Cancer (“AJCC”) 1998 staging system. The subjects had a non-operated, previously untreated Stage III, Stage IVA, or Stage IVB LA-SCCHN (according to the American Joint Committee on Cancer 1998 staging system with T≥2, N0-3 M0).

Escalating doses of Debio 1143 were added to CRT. Debio 1143 was administered orally daily for 14 days every three weeks (on days 1-14, 22-35 and 43-56). The starting dose of Debio 1143 in this combination study was 100 mg/day over 14 days every 21 days. The total administered dose of Debio 1143 at the starting dose level was 1400 mg per cycle. Treatment with Debio 1143 was permanently discontinued if radiotherapy (RT) was prematurely discontinued. Debio 1143 treatment continued if cisplatin was discontinued.

Intravenous cisplatin was administered over 1 hour at a dose of 100 mg/m² (for 3 cycles—Day 2, Day 23 and Day 44). Cisplatin was given before irradiation. Hydration and antiemetics were delivered according to standards of care. Standard fraction radiotherapy to the primary tumour was delivered daily for 5 days per week to a total dose of 70 Gy in 2 Gy daily fractions over 7 weeks. Nodal areas not clinically involved by tumour received a total dose of 50 Gy.

Radiotherapy was interrupted for a maximum of 10 days, totally, to allow resolution/improvement of radio-chemo toxicities.

Most subjects in the study were very advanced stage, had a heavy smoking history and nearly 90% of OPCs were HPV-p16 negative status.

The maximum tolerated dose of Debio 1143 was 200 mg. Debio 1143 significantly improves efficacy in high risk LA-SCCHN subjects versus CRT+placebo. Debio 1143 also exhibited a predictable and manageable safety profile without substantial additional toxicity to standard CRT. Good overall compliance to the treatment with Debio 1143 was observed.

### Example 2

A double-blind randomized placebo-controlled Phase II study was conducted. In the study, 96 subjects with locally advanced squamous cell cancer of the head and neck were randomly assigned to a placebo+CRT arm or a Debio 1143+CRT arm. 96 patients were randomized 95 were treated. The subjects assigned to the Debio 1143 arm received 200 mg/day orally daily for 14 days every three weeks (on days 1-14, 22-35 and 43-56 (up to three cycles every three weeks)). All subjects received 100 mg/m² cisplatin on day 2 of each three-week cycle (up to three cycles) concomitantly with radiotherapy (RT). The subjects also received standard fractionation RT with 2 Gy/daily on five days/week for seven weeks up to 70 Gy delivered to the gross-tumor volume (GTV) and 50 Gy to eligible lymph nodes loco-regionally.

The primary endpoint to the study was locoregional control rate (LRC) at 18 months after CRT. Key secondary endpoints included PFS, duration of LRC, overall survival, odds ratio (OR) and CR at 3 and 6 months after CRT completion.

The main inclusion criteria were as follows:
Previously untreated, inoperable (unresectable) Stage III, Stage IVA, or Stage IVB as per AJCC TNM staging version 7.0 (2010)
LA-SCCHN of the oral cavity, hypopharynx, larynx, or oropharynx (HPV/p16 both negative or positive)
Heavy smoking history (>10 pack-years)
Negative HIV/HBV/HCV
Performance Status by Eastern Cooperative Oncology Group (ECOG)=0 or 1.

The main exclusion criteria were as follows:
Non-compensated liver cirrhosis (Child-Pugh class C)
>75 years
Non-cisplatin eligible.

The patient demographics are shown in Table 1.

TABLE 1

| | Debio 1143 + | | Placebo + | |
|---|---|---|---|---|
| | CRT (N = 48) | | CRT (N = 48) | |
| Age: median (range) | 57 | (39-70) | 59 | (46-74) |

| | | |
|---|---|---|
| Sex: % male/% female | 77.1/22.9 | 85.4/14.6 |
| ECOG (at baseline) % | 56.3/41.7 | 56.3/43.8 |
| for grade 0/1 | | |

Primary tumor localization

| | | | | |
|---|---|---|---|---|
| Hypropharynx | 7 | (14.6%) | 10 | (20.8%) |
| Larynx | 8 | (16.7%) | 2 | (4.2%) |
| Oral cavity | 2 | (4.2%) | 3 | (6.3%) |
| Oropharynx (OPC) | 31 | (64.6%) | 33 | (68.7%) |
| HPV/p-16 negative (% of OPC) | 28 | (90.3%) | 28 | (84.8%) |
| HPV/p-16 positive (% of OPC) | 3 | (9.7%) | 5 | (15.2%) |

TNM staging

| | | | | |
|---|---|---|---|---|
| Stage III | 7 | (14.6%) | 8 | (16.7%) |
| Stage IVA | 35 | (72.9%) | 32 | (66.7%) |
| Stage IVB | 6 | (12.5%) | 8 | (16.7%) |

Smoking history

| | | | | |
|---|---|---|---|---|
| Total pack - years: | 40 | (15-104) | 40 | (11-90) |
| median (range) | | | | |

Alcohol history

| | | | | |
|---|---|---|---|---|
| Current drinker | 26 | (54.2%) | 31 | (64.6%) |
| Average drinks per week: | 21 | (1-50) | 21 | (3-140) |
| median (range) | | | | |

Baseline characteristics between the two arms were well balanced. Over 80% of OPC are HPV/p16 negative. All subjects were heavy smokers and over 80% had Stage IV LA-SCCHN. The subjects were also high alcohol consumers.

As shown in Table 2, overall treatment exposure between the two arms are comparable. Also, both arms received identical cisplatin dose intensity and have comparable radiotherapy doses. The subjects exhibited good compliance to Debio 1143 (similar to those receiving placebo). The data provided in Table 2 are provided as median (range).

TABLE 2

| | Debio 1143 + | | Placebo + | |
|---|---|---|---|---|
| | CRT (N = 48) | | CRT (N = 47) | |
| Number of administrations | | | | |
| Planned = 42 | 39 | (1; 42) | 41 | (1; 42) |
| Exposure to Debio 1143 | | | | |
| or placebo mg | | | | |
| Total cumulative dose | 7425 | (200; 8400) | 8200 | (200; 8400) |
| Relative dose intensity ≥70% | 35 | (72.9%) | 38 | (80.9%) |
| Treatment delay days | 2 | (1; 7) | 2 | (1; 8) |
| CDDP | | | | |
| Cumulative dose in mg/m² | 288 | (99; 300) | 288 | (100; 300) |
| Relative dose intensity | 96.9% | | 96.9% | |
| (% of total planned dose) | | | | |
| RT | | | | |
| Patients receiving ≥70% | 44 | (91.7%) | 41 | (87.2%) |
| of planned dose | | | | |
| Patients receiving less | 5 | (10.4%) | 6 | (12.8%) |
| than 66 Gy | | | | |

26 of the subjects assigned to the Debio 1143+CRT arm completed the two-year follow-up (22 discontinued because of disease progression (10), withdrawn consent (2) adverse events (3), death (1), or other (4)). In comparison, only 14 subjects assigned to the placebo+CRT arm completed the two-year follow-up (34 discontinued because of disease progression (16), withdrawn consent (4), adverse events (4), deaths (8), or other (4).

As shown in Table 3, the primary endpoint, loco-regional control rate at 18 months after CRT, was met (an improvement of more than 20%).

TABLE 3

| | Debio | Placebo + | |
|---|---|---|---|
| | 1143 + CRT | CRT | Debio 1143 vs. |
| | N = 48 | N = 48 | Placebo + CRT |
| Locoregional Control-rate | 54.2 | 33.3 | Δ:20.8 |
| At 18 months (%) | (39.2; 68.6) | 20.4; 48.4) | (1.4; 40.2) |
| (95% Cl) | | | |
| Odds Ratio (95% Cl) | | | 2.69 |
| (Debio 1143 vs Placebo) | | | (1.13; 6.42) |
| p-value | | | 0.026 |

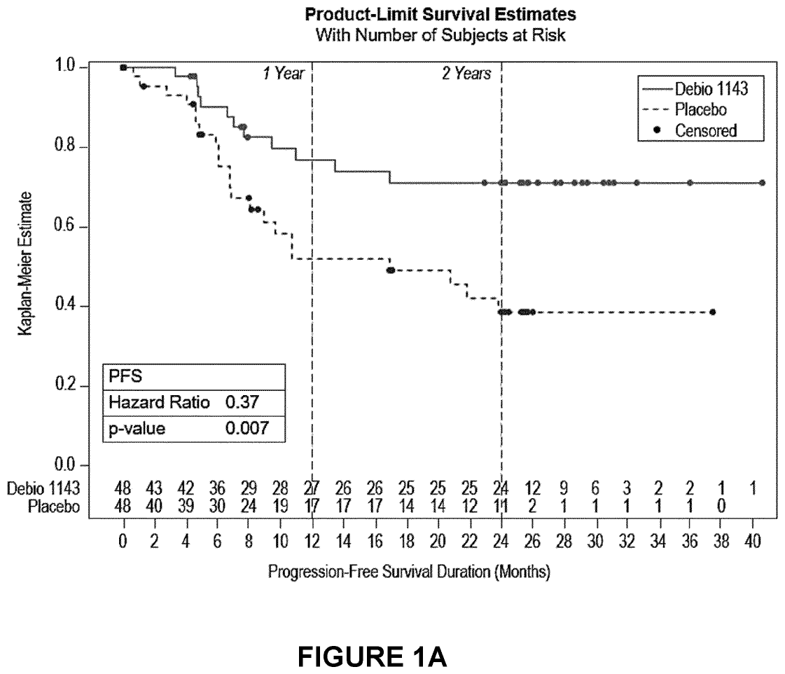

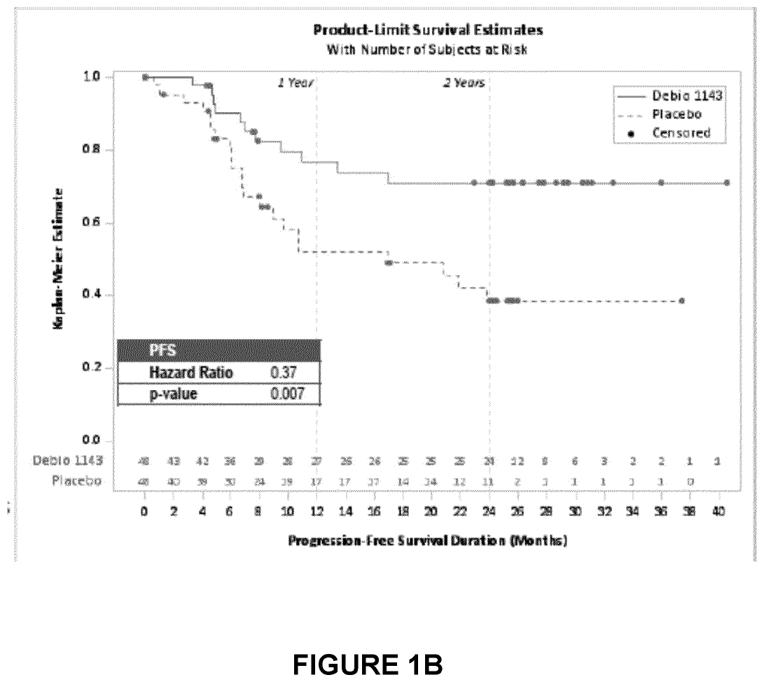

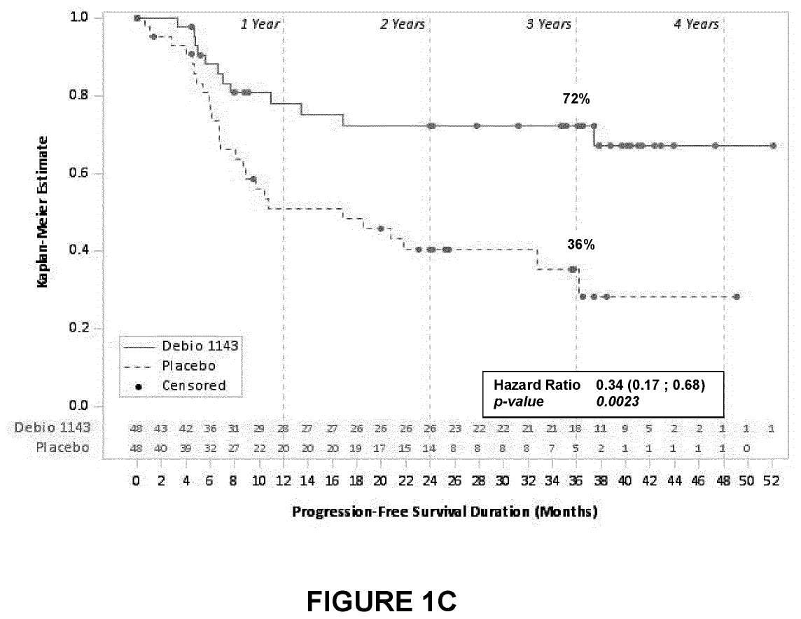

As shown in FIG. 1, highly significant progression-free survival improvement in the subjects administered Debio 1143 was also observed.

Debio 1143 exhibited similar safety as compared to placebo, except for Grade 3 dysphagia, mucositis, anemia (all of which were consistent with radiosensitizing (RS) effect). Grade 4 adverse effects were limited and similar. No treatment-related death were observed in Debio 1143-treated patients while two grade 5 events occurred in the placebo arm. Late toxicities (onset >60 days after CRT end) were very similar and not increased in Debio 1143 arm. Safety with Debio 1143 was predictable and manageable without increases in life-threatening toxicities nor late toxicities.

The results from the study were positive because the primary endpoint has been met (statistically significant improvement of 21% in LRC at 18 months after CRT (odds ratio (OR) 95%; p=0.026). A highly significant and clinically compelling PFS improvement (HR 0.37; p=0.007) was observed. A consistent positive trend towards improved OS (HR 0.65; p=0.243) was also observed, although median OS was not yet reached in either arm.

A combination of Debio 1143 with CRT in SCCHN showed encouraging results on the high-risk population (heavy smokers, high alcohol consumption, late-stage (Stage III, Stage IVA, or Stage IVB), and/or oropharynx that is HPV/p-16 negative)). Based on these results, further investigation in a Phase III study is warranted.

### Example 3

### Study Design and Participants

A multicentre, phase 2, double-blind, randomised, placebo-controlled study was conducted to evaluate the safety and efficacy of oral Debio 1143 at 200 mg/day on Days 1-14 of 21-Day cycles combined with concomitant cisplatin-based chemoradiotherapy in patients with LA-SCCHN. Male and female patients were eligible if they were aged 18 to 75 years with histologically confirmed LA-SCCHN and fulfilled the following criteria: previously untreated, stage III, IVa or IVb (according to the American Joint Committee on Cancer 2010 staging system [AJCC TNM v7.0 2010]) of one or more of the following sites: oral cavity, oropharynx, hypopharynx and larynx and with T 2-4, N0-3, M0 measurable disease according to Response Evaluation Criteria in Solid Tumours (RECIST), version 1⋅1. Patients were to have a smoking history of more than 10 pack-years, and no medical history of hepatitis B or hepatitis C. For patients with oropharyngeal cancer, tumour HPV-status was determined by the presence of p16 using immunohistochemistry. Other inclusion criteria included: Eastern Cooperative Oncology Group (ECOG) performance status of 0 or 1, no clinically significant cardiac disease and no clinically significant hearing impairment that would have contraindicated the use of chemotherapy with HD-cisplatin.

Exclusion criteria included nasopharyngeal, paranasal sinuses, nasal cavity tumours or thyroid cancers, squamous cell cancer involving cervical neck nodes but from unknown primary site, unknown lymph node status, metastatic disease, any prior or current treatment for invasive head and neck cancer of any kind (including, but not limited to, prior tyrosine kinase inhibitors, prior neoadjuvant therapy, prior surgical resection, or use of any investigational agent), weight loss of >10% during the previous month, non-compensated liver cirrhosis, gastro-intestinal disorders that could affect drug absorption, concurrent treatment with any other systemic anticancer therapy or concomitant treatment with any drug on the prohibited medication list, history of uncontrolled or symptomatic angina, arrhythmias, or congestive heart failure, history of another malignancy within the last 5 years with the exception of completely resected basal or squamous cell skin cancer or successfully treated in-situ carcinoma, history of non-invasive lesion or in-situ carcinoma, including in the head and neck region that was successfully treated with surgery, peripheral neuropathy (grade 2 or above), clinical hearing loss, history of allergic reactions to appropriate antiemetics, human immunodeficiency virus infection. The protocol was approved by the institutional review boards or ethics committees of all participating centres. The study was carried out in accordance with the protocol, the principles expressed in the Declaration of Helsinki, and applicable regulatory requirements. All patients provided written informed consent in advance of study-specific procedures.

### Randomisation and Masking

Randomisation (1:1) was performed by a stochastic minimisation technique according to the following stratification factors: node involvement (N0-N1 vs N2-N3) and primary tumour site (oropharynx vs others), as well as HPV-16 status (positive vs negative, as determined by p16 immunohistochemistry) within patients with an oropharyngeal primary tumour site. Randomisation codes were generated by an external supplier and the investigators and study team remain blinded until the final analysis.

### Procedures

Patients received either Debio 1143 or placebo (20 mg/mL, active pharmaceutical ingredient solution in single-dose glass vials containing 10 mL of solution (200 mg Debio 1143 per vial or placebo) orally or via a feeding tube according to nutritional status and/or swallowing capability) daily on days 1 to 14 of 21 day treatment cycles, for up to 3 cycles (taken fasted 1 hour before or 2 hours after a meal). Cisplatin 100 mg/m² was administered intravenously over 60 min before the irradiation fraction, once in every cycle for up to 3 cycles (on days 2, 23 and 44) and ≥30 min (but no more than 3 hours) after Debio 1143/placebo. Conventional fractionated intensity modulated radiotherapy (IMRT) was delivered to the gross tumour volume (GTV, [primary tumour and involved nodes]), to a total dose of 70 Gy in 2 Gy daily fractions, for 5 days per week over 7 weeks. Primary and neck nodal areas with no tumour involvement (elective/prophylactic irradiation areas) received a total dose of 50 Gy. In accordance with the European Society of Medical Oncology guidelines, patients received prophylactic treatment with granisetron or palonosetron, dexamethasone and aprepitant, prior to and after cisplatin to prevent renal damage and emesis.

Tumour assessments were performed at screening when patients were evaluated and staged using (AJCC TNM v7⋅0 (2010). A computerised tomography (CT) scan and/or magnetic resonance imaging (MRI) of the head and neck region, and chest CT and optional fluorodeoxyglucose-positron emission tomography (¹⁸FDG-PET) scan were also performed. During the study, investigator tumour response assessments were performed according to RECIST (v1⋅1) guidelines. ¹⁸FDG-PET scan imaging, while not mandatory, was permitted if there was doubt about whether residual disease existed, or to identify new lesions which were then to be confirmed by CT or biopsy/surgery. The end of treatment visit was 10 days after the final treatment (±3 days). A safety follow-up was scheduled for 30-40 days after the final treatment, and the first efficacy follow-up 11 weeks (1 week), after the end of treatment visit, followed by re-evaluation every 3 months until 2 years after randomisation. Patients who were still ongoing at that point were asked to enter the extended follow-up, which continued for all patients until the last patient has reached 3 years since randomisation. During the extended follow-up, the frequency of assessments was 3- to 6-monthly intervals. Patients continued evaluation of disease status and late-onset toxicity until disease progression or a switch to another systemic anticancer therapy.

Where an adverse event could reasonably be attributed to chemoradiotherapy, adjustments to chemoradiotherapy were attempted before adjusting the Debio 1143/placebo dose. In patients experiencing Debio1143-related toxicities requiring dose reduction, Debio 1143/placebo was reduced by 50 mg at each occurrence; a maximum of two dose reductions were permitted. If a further dose reduction was indicated, Debio 1143/placebo was discontinued; re-escalation was not permitted.

Medical history and demographics were collected at screening. Physical examination was conducted at screening, on day 1 of each cycle, at the end of study visit, at the safety follow-up visit and at each subsequent efficacy follow-up visit. Adverse events and co-medication were monitored throughout the study from signature of informed consent (28 days prior to the first dose) until 30 days after the last dose of study drug. Adverse events were graded according to Common Terminology Criteria for Adverse Events (NCI CTCAE) version 4⋅03. All information pertaining to unusual manifestations or adverse events was collected by the investigator at each visit. The investigator was required to notify the sponsor or delegate of any serious adverse event, irrespective of reason, within 24 hours of being informed of its occurrence. Quality control of contouring and dosimetry was performed by independent expert radiation oncologists. Late toxicities were defined in the protocol and monitored throughout the efficacy follow-up period until the end of study visit.

### Outcomes

The primary endpoint was the proportion of patients achieving locoregional control at 18 months from the end of chemoradiotherapy, defined as the documented absence of locoregional failure up to and including that time point. Locoregional failure was recorded by the investigator, either according to the RECIST criteria or based on the investigator's blinded clinical assessment and confirmed by biopsy. Key secondary endpoints were: progression-free survival, defined as the time from randomisation to death (from any cause) or disease progression; duration of locoregional control, defined as the time from the end of chemoradiotherapy to occurrence of locoregional relapse; time to distant relapse, defined as the time from the end of chemoradiotherapy to occurrence of distant relapse, and overall survival, defined as the time from randomisation to death from any cause. Other secondary endpoints included complete response rate six months after treatment, best overall response and response rate at three and six months after treatment.

### Results

Between 25 Jan. 2016 and 24 Apr. 2017, 96 patients were enrolled and randomly assigned to receive Debio 1143 plus chemoradiotherapy (n=48) or placebo plus chemoradiotherapy (n=48); these patients constituted the intention-to-treat population. Only one patient, from the placebo/chemoradiotherapy group, did not receive study treatment thus, there were 95 patients in the safety population. Baseline characteristics were well balanced between the two treatment groups; in the Debio 1143 group there were eight (17%) patients with primary tumours of the larynx, versus three (4%) in the placebo group, and three (6%) patients with HPV-positive oropharyngeal cancer, versus five (10%) in the placebo group, although disease stages were well balanced, as were tumour size and lymph node involvement. All patients had a heavy smoking history. Over half of the patients were current drinkers at baseline, with a high alcohol consumption (median of average drinks per week being 21).

The data cut off was 15 Jul. 2019. Treatment exposure for Debio 1143/placebo was comparable between groups; median total cumulative dose of cisplatin was 288 mg/m2 in both groups, and 42 of 48 (88%) patients received two or more cycles of cisplatin in the Debio 1143 group versus 39 of 47 (83%) in the placebo group. The median total cumulative dose of radiotherapy (IMRT) delivered to the gross tumour volume (GTV) was 70 Gy (Interquartile Range IQR; 70-70) in both groups and 51.8 Gy (IQR; 50-54.4 [Debio 1143 group]50-51.8 [placebo group]) to the elective lymph nodes regionally, in both groups. Seven of 48 (15%) patients discontinued Debio 1143, while six of 48 (13%) patients discontinued the placebo.

At the 18-month timepoint, the pre-specified primary endpoint of locoregional control rate was significantly higher in the Debio 1143 group. Locoregional control was achieved and ongoing in 26 of 48 (54%) patients in the Debio 1143 group (95% CI: 39-69) versus 16 of 48 (33%) patients in the placebo group (95% CI: 20-48), (odds ratio 2.69; 95% CI: 1.13-6.42, p=0.026). Kaplan-Meier estimates of locoregional control at 18 months after the end of chemoradiotherapy were 78% in the Debio 1143 group versus 67% in the placebo group. The median duration of locoregional control was not reached in either treatment group (hazard ratio [HR]0.53 [95% CI 0.22-1.30], p=0.165).

Progression-free survival was a key secondary endpoint, median progression-free survival for patients in the Debio 1143 group was not reached at data cut-off and was 16.9 months for the placebo group ([HR]0.37; 95% CI 0.18-0.76; p=0.007); the proportion of patients who were progression-free in the Debio 1143 group at 24 months was 72% (95% CI 56%-84%) compared with 41% (95% CI 0.25-0.55) in the placebo group (p=0.003). Consistent with PFS, there was a positive trend towards improved overall survival in the Debio 1143 group ([HR]0.65; 95% CI 0.32-1.33; p=0.243). Median overall survival was not reached in either group by 24 months. Additional blinded follow-up is ongoing.

First tumour evaluation occurred at approximately 11 weeks after chemoradiotherapy: a complete response in 17 of 48 (35%) patients was observed in both groups, partial response in 13 of 48 (27%) in the Debio 1143 group versus 15 of 48 (31%) in the placebo group. At the following tumour evaluation, six months post chemoradiotherapy, there was a complete response in 25 of 48 (52%) patients in the Debio 1143 group versus 18 of 48 (38%) in the placebo group, and partial response in seven of 48 (15%) in the Debio 1143 group versus five of 48 (10%) in the placebo group.

Overall the addition of Debio 1143 to chemoradiotherapy was well tolerated and consistent with the safety profile of chemoradiotherapy alone. All patients experienced at least one treatment related adverse event. Adverse events of at least grade 3 were reported in 41 of 48 (85%) patients in the Debio 1143 group and 41 of 47 (87%) patients in the placebo group. The most common events were dysphagia, mucosal inflammation, and anaemia, with grade 3 events being more frequent in the Debio 1143 group. In the Debio 1143 group nine (19%) patients experienced grade 4 events, none experienced grade 5 events. In the placebo group, grade 4 events were reported in 11 (23%) patients and grade 5 events occurred in two (4%) patients (1 multiple organ failure, 1 asphyxia). Serious treatment-emergent adverse events were recorded in 58 patients (30 [63%] in the Debio 1143 group versus 28 [60%] in the placebo group).

Treatment-emergent adverse events leading to dose reductions of Debio 1143 were reported in nine of 48 (19%) patients; the most common was alanine aminotransferase increase in five (10%) patients. Five (11%) patients had treatment-emergent adverse events leading to dose reduction of placebo, the most common was vomiting in two (4%) patients.

Debio 1143 treatment did not increase the frequency or severity of cisplatin-associated adverse events (renal insufficiency, febrile neutropenia, thrombocytopenia, peripheral sensory neuropathy or severe vomiting) with the exception of grade 1-2 tinnitus in 15 of 48 (31%) versus 10 of 47 (21%) in the placebo group. Grade 3 increases in aspartate aminotransferase and alanine aminotransferase were higher in the Debio 1143 group. However, no trend between transaminases increase and Debio 1143 exposure (in terms of area under the curve) was clearly identified. Of note, there were no grade 4 transaminase increases in the Debio 1143 group and no grade 3 bilirubin increases. There was a higher incidence of grade 3 anaemia and a higher incidence of grade 4 neutropenia in the Debio 1143 group. However, the frequencies of grade 3 to 4 febrile neutropenia reported as a treatment-emergent adverse event were similar between the treatment groups. Overall, late toxicity was balanced and grade 3 or higher late toxicity was relatively rare in both treatment groups. In the Debio 1143 group, 14 patients died versus 17 in the placebo group. No deaths were considered to be treatment related.

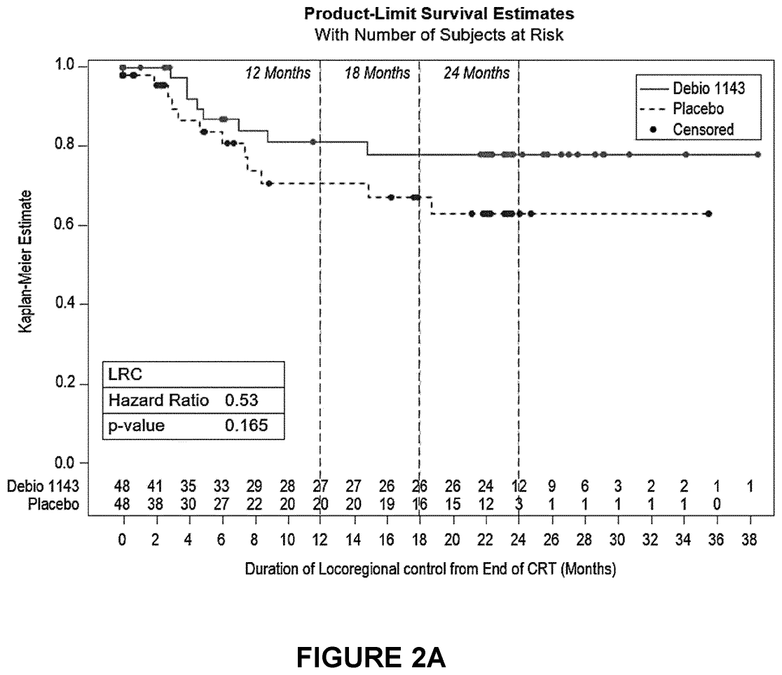

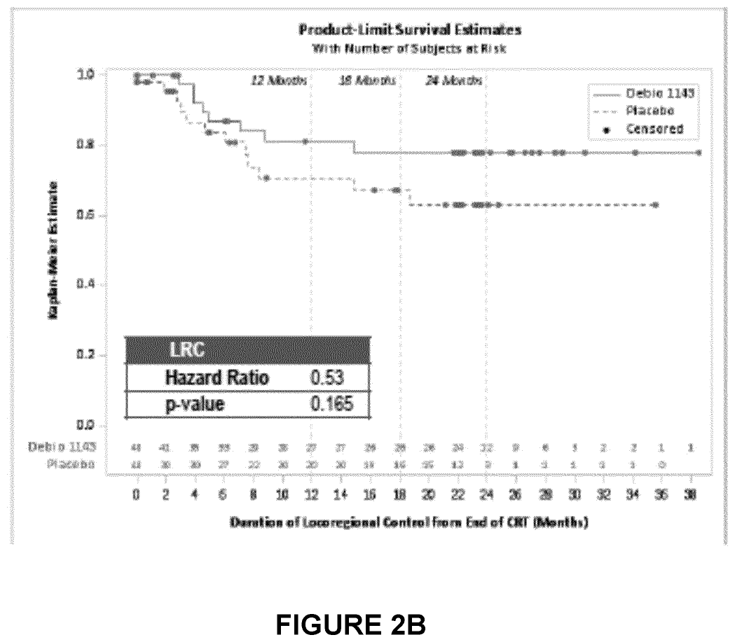

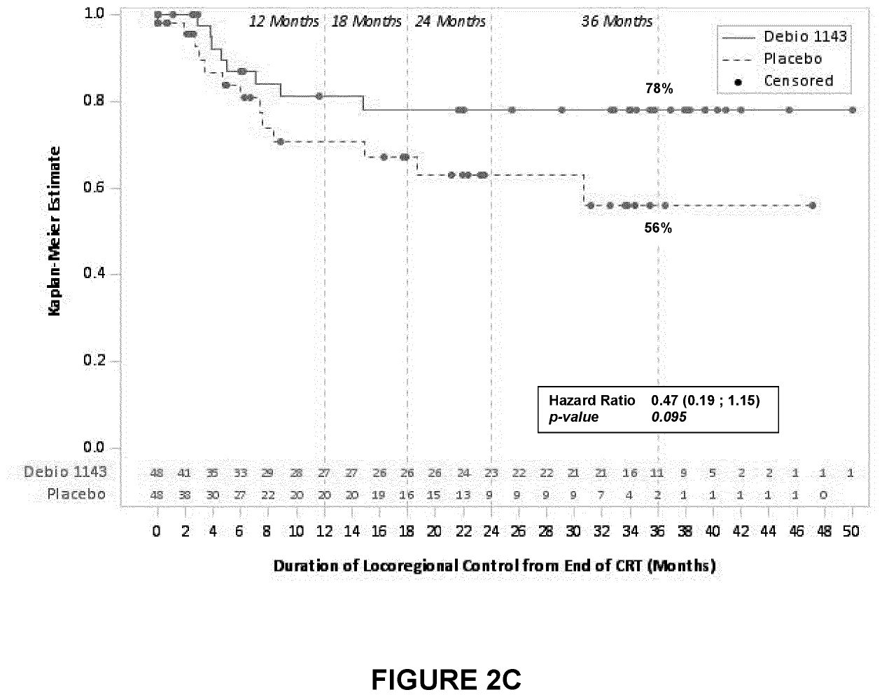

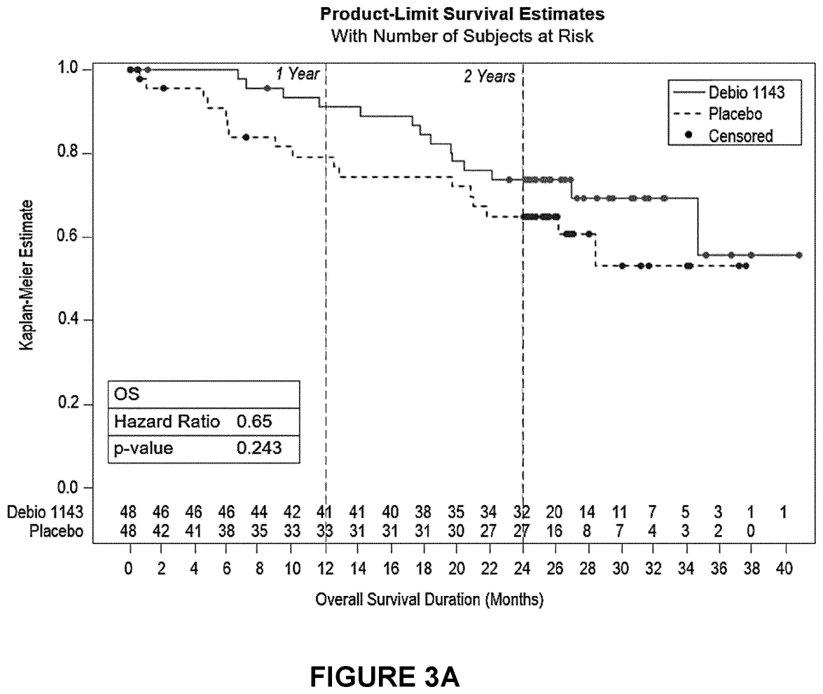

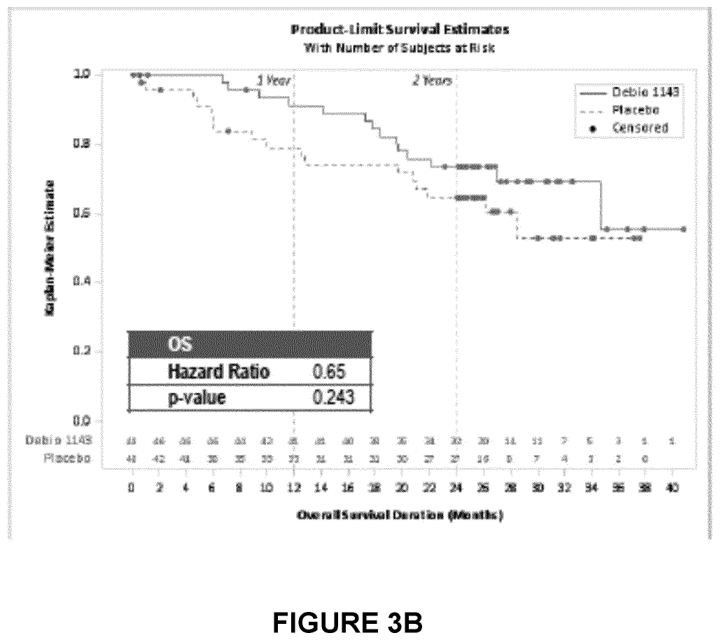

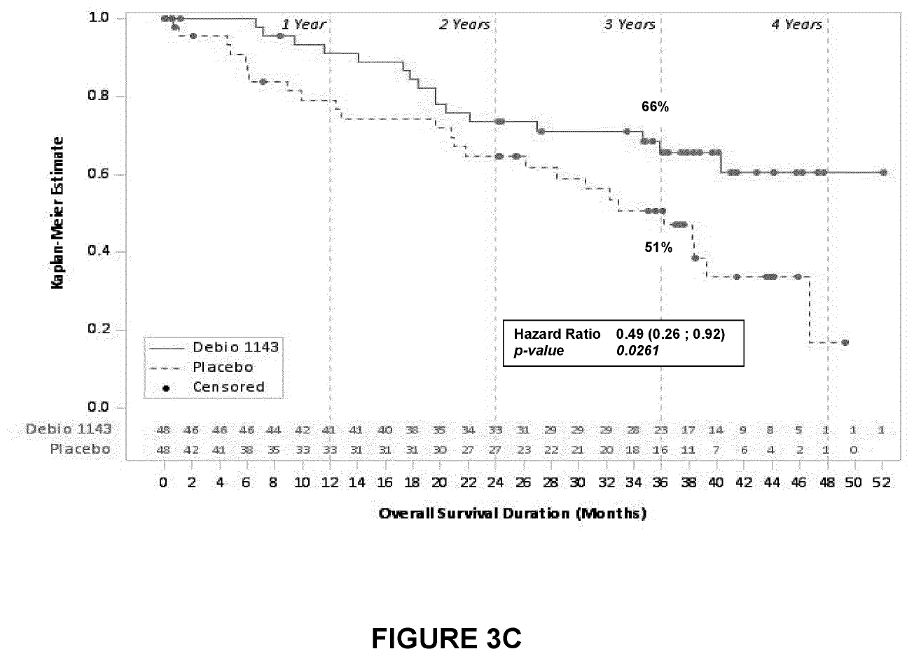

As of 21 Jul. 2020, the median follow-up was 33 months. Debio 1143 combined with CRT showed a statistically significant improvement in OS vs placebo by reducing the risk of mortality by 51% (HR=0.49, [95% CI: 0.26-0.92], p=0.0261). The 3 years OS rate was 66% (95% CI: 49-78) in the Debio 1143 arm versus 51% (95% CI: 34-65) in the placebo arm; the median OS was not reached with Debio 1143 vs. 36.1 months with placebo (95% CI: 21.8-46.7) (FIG. 3C). Statistically significant improvement in PFS was demonstrated, reducing the risk of disease progression or death by 66% (HR=0.34 [95% CI, 0.17-0.68], p=0.0023) and improving probability of PFS at 36 months to 72% in the Debio 1143 arm compared to 36% in the placebo arm (FIG. 1C). Kaplan-Meier estimates of locoregional control at 36 months after the end of chemoradiotherapy were 78% in the Debio 1143 group versus 56% in the placebo group. The median duration of locoregional control was not reached in either treatment group (hazard ratio [HR]0.47 [95% CI 0.19-1.15], p=0.095) (FIG. 2C).

The predictable and manageable safety profile observed with Debio 1143+CRT after 2 years remained unchanged after 3 years.

These results with extended follow-up confirm those reported at 24 months, showing in addition a statistically and clinically significant OS benefit by adding Debio 1143 to standard CRT.

### Example 4

A study to determine an efficacy evaluation of Debio 1143 versus matched placebo when added to standard platinum-based concomitant chemoradiotherapy in high risk LA-SSCHN subjects will be conducted. Approximately 700 adult male and female subjects with inoperable, previously untreated, high risk histologically confirmed LA-SCCHN (Stage III, Stage IVA, or Stage IVB, as determined by American Joint Committee on Cancer (AJCC) staging) will be included. Subjects with oropharynx primary site must either be HPV-negative by p16 immunohistochemistry or can be HPV-positive if they have a positive smoking history of 10 pack-years or more or the tumor is T4b and/or N3 and/or extracapsular node extension positive per AJCC staging.

Debio 1143 at 200 mg/day, once daily, on Days 1-14 every 3 weeks as an oral solution. If necessary, the oral solution will be administered via a nasogastric tube, percutaneous endoscopic gastrostomy, or percutaneous endoscopic jejunostomy tube. Matched placebo, once daily, on Days 1-14 every 3 weeks as an oral solution will be administered via a nasogastric tube, percutaneous endoscopic gastrostomy, or percutaneous endoscopic jejunostomy tube if necessary.

In variant 1, cisplatin (commercial formulation) will be administered starting on Day 2 Cycle 1, 1 hour after the Debio 1143 intake, administered at either high-dose (100 mg/m² every 3 weeks) or at low-dose (40 mg/m² weekly), as an intravenous (IV) infusion, as per institutional guidelines, with pre and post-infusion hydration±mannitol and/or diuretics as follows.
Low dose cisplatin regimen: The cisplatin is administered on Days 2, 9 or 10, or 16 or 17 of Cycles 1, 2, and 3 of the first regimen.
High dose cisplatin regimen: The cisplatin is administered on Day 2 of Cycles 1, 2, and 3 of the first regimen.

In variant 2, cisplatin (commercial formulation) will be administered starting on Day 1 Cycle 1, 1 hour after the Debio 1143 intake, administered at either high-dose (100 mg/m² every 3 weeks) or at low-dose (40 mg/m² weekly), as an intravenous (IV) infusion, as per institutional guidelines, with pre and post-infusion hydration±mannitol and/or diuretics.

In both variants, subjects with glomerular filtration rate (GFR) between 30-59 ml/min/1.73 m² body surface area (BSA), who are not eligible to receive cisplatin with concurrent radiotherapy (RT), may start with the equivalent carboplatin schedule. Carboplatin (commercial formulation) is administered at a high-dose (AUC=4.5-5, every 3 weeks) or at a low-dose (AUC=1.5-2 weekly), as an IV infusion over at least 30 minutes, per institutional guidelines.

Conventional fractionation RT, 2.0 Gy/fraction, 5 fractions per week, will be delivered to the primary tumor site and locoregionally involved areas over 7-9 weeks (up to a maximum of 70 Gy (in the above-mentioned variant 1) or a maximum of 72 Gy (in the above-mentioned variant 2)), by either intensity modulated radiotherapy (IMRT) or 3D conformal RT planning primary systematic target volume (STV). Nodal areas without clinical tumor involvement will receive a total dose of up to 50 Gy.

In both variants, Debio 1143/matched placebo treatment will be administered under fasting conditions (for at least 1 hour before administration and for at least 2 hours after) in combination with CRT, starting on the same day as platinum and radiotherapy initiation.

Subjects will receive Debio 1143/matched placebo and platinum treatment for the duration of the concomitant RT (7-9 weeks). At the end of the RT, platinum will be stopped and Debio 1143/placebo will be continued for two additional 3-week cycles as a single agent.

Debio 1143/placebo will be administered on Days 1 to 14 of 3-week cycles, with three cycles of Debio 1143/placebo with concomitant platinum during 7-9 weeks of RT, followed by two cycles of Debio 1143/placebo alone, with an End of Treatment (EOT) visit 2 weeks after the last Debio 1143/placebo dose on Day 14 of Cycle 5. The treatment period is thus considered to be up to 16 weeks. Treatment will be administered until occurrence of progressive disease (PD) or unacceptable toxicity.

### Example 5

A study of Debio 1143 for administration in combination with standard platinum-based concomitant chemoradiotherapy as upfront treatment in high-risk LA-SCCHN subjects may be conducted.

Approximately 600 subjects will be randomized into one of two-arms comparing the efficacy and safety of Debio 1143 versus matched placebo when administered in combination with platinum-based CRT in subjects with inoperable, previously untreated, high risk LA-SCCHN.

Eligible subjects will be randomized in a 1:1 ratio to receive Debio 1143+CRT (Arm A) or matched placebo+CRT (Arm B), with stratification by region, ethnicity, primary tumor site, lymph node involvement, and hemoglobin level.

The primary objective is to demonstrate superior efficacy of Debio 1143 versus matched placebo when added to CRT in high-risk LA-SCCHN subjects, as assessed by the investigator, with the primary endpoint of event-free survival (EFS), as assessed by the investigator. The key secondary endpoint is EFS, per blinded independent review committee (BIRC) and OS

The subjects will have inoperable, previously untreated, high-risk, histologically confirmed LA-SCCHN (Stage III, Stage IVA or Stage IVB, per AJCC) (e.g., oral cavity, hypopharynx, larynx, oropharynx). Subjects with oropharynx primary site must either be HPV-negative by p16 immunohistochemistry.

The subjects will be administered Debio 1143 oral solution at 200 mg/day, once daily, on Days 1-14 of a 3-week cycle, for up to 3 cycles, in combination with CRT. Debio 1143 will be administered from the same day as radiotherapy (Day 1). If necessary, the oral solution will be administered via a nasogastric tube, percutaneous endoscopic gastrostomy (PEG), or percutaneous endoscopic jejunostomy tube. The subjects randomized to the placebo arm will be administered a matched placebo oral solution similarly to those receiving Debio 1143.

Debio 1143 and placebo treatment will be administered under fasting conditions (for at least 1 hour before start of administration and for at least 2 hours after end of administration).

High-dose cisplatin (commercial formulation) (100 mg/m²) on Day 1 (variant 1) or Day 2 (variant 2) of a 3-week cycle, for up to 3 cycles, as an intravenous (IV) infusion, according to institutional guidelines, with pre and post-infusion hydration±mannitol and/or diuretics.

Subjects with glomerular filtration rate (GFR) between 30-59 ml/min/1.73 m² body surface area (BSA) after Cycle 1 who are not considered eligible to receive cisplatin with concurrent radiotherapy in subsequent cycles, may continue for Cycles 2 and 3 with the equivalent carboplatin schedule (AUC=4.5-5) on Day 1 (according to the above-mentioned variant 1) or Day 2 (according to the above-mentioned variant 2) of the 3-week cycle, as an IV infusion over at least 30 minutes, per institutional guidelines.

Platinum will be administered 30 minutes after Debio 1143/placebo and before radiotherapy.

Conventional fractionation RT, 2.0 Gy/fraction, 5 fractions per week (5/7 days), to the primary tumor site and locoregionally involved areas, will be delivered over 7-9 weeks (up to a maximum of 70 Gy), by either intensity modulated radiotherapy or 3D conformal radiotherapy planning primary systematic target volume. Nodal areas without clinical tumor involvement will receive a total dose of up to 50 Gy.

### Example 6

A pivotal study aiming to generate data to support regulatory filings of Debio 1143 for administration in combination with standard platinum-based concomitant chemoradiotherapy as upfront treatment in high-risk LA-SCCHN subjects will be conducted.

Approximately 650 subjects with unresectable, previously untreated, high-risk, histologically confirmed LA-SCCHN (stage III, IVA or IVB, per American Joint Committee on Cancer [AJCC]/TNM staging, 8th ed.) will be randomized in a 1:1 ratio to receive Debio 1143+CRT (Arm A) or matched placebo+CRT (Arm B) to compare the efficacy and safety of Debio 1143 versus matched placebo. Subjects with oropharynx (OPC) primary site must be HPV-negative by p16 immunohistochemistry.

The primary objective is to demonstrate superior efficacy of Debio 1143 versus matched placebo when added to CRT in high-risk LA-SCCHN subjects.

The subjects will be administered Debio 1143 oral solution at 200 mg/day, once daily, on Days 1-14 of a 3-week cycle (q3w), for up to 3 cycles, in combination with CRT for up to 3 cycles. Debio 1143 will be administered from the same day as radiotherapy (Day 1). After CRT end, Debio 1143 monotherapy at 200 mg/day Days 1-14 q3w will be continued for up to 3 additional cycles, in a double blinded fashion. If necessary, the oral solution will be administered via a nasogastric tube, percutaneous endoscopic gastrostomy (PEG), or percutaneous endoscopic jejunostomy tube. The subjects randomized to the placebo arm will be administered a matched placebo oral solution similarly to those receiving Debio 1143.

Debio 1143 should be administered orally early in the morning, following a fast of at least 2 hours after any meal. Patients should fast for at least 1 hour after dosing. Water is permitted freely.

High-dose cisplatin (commercial formulation) (100 mg/m²) on Day 2 q3w, for up to 3 cycles, as an intravenous (IV) infusion over at least 90 minutes, according to institutional guidelines, with pre and post-infusion hydration±mannitol and/or diuretics. Standard antiemetics will be administered as per institutional guidelines; 5-HT3 receptor antagonists such as granisetron or palanosetron are allowed, however ondansetron should be avoided.

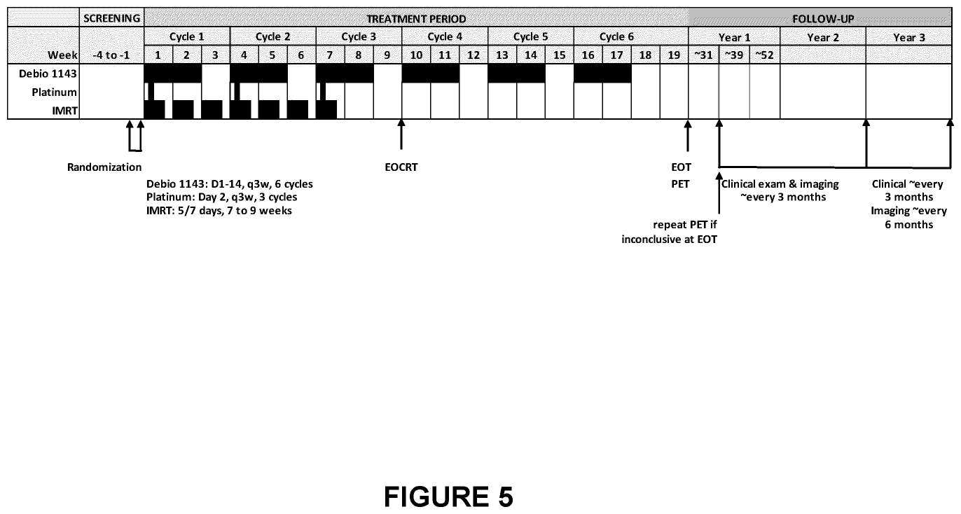

On days when RT and CT are to be administered (see FIG. 5), Debio 1143 or matched placebo should be administered between 3 h to 30 minutes before CT, and CT should be administered before RT.

On days when RT only is to be administered (see FIG. 5), Debio 1143 or matched placebo should be administered before RT.

If a dose administration of Debio 1143 is delayed and the reason for this delay is NOT toxicity, the patient may take the dose no later than 6 hours after normal intake time. If the delay exceeds 6 hours, the patient must wait for the next scheduled intake. Debio 1143 doses/matched placebo that were omitted a specific day should not under any circumstances be administered any other day in addition to the scheduled intake.

No additional dose should be re administered after vomiting; the patient must wait for the next scheduled intake.

After a treatment interruption NOT caused by an Adverse Event related to Debio 1143/matched placebo, the treatment can be resumed at the same dose only if the patient did not require more than 1 treatment interruption per cycle AND the interruption lasted maximum 3 consecutive days. In case of nausea, vomiting, and diarrhea, dose adjustments should be applied only after optimal supportive therapy.

### General Rules for Debio 1143/Placebo Administration are:

Only two sequential dose reductions of 2.5 ml per step (corresponding to 50 mg/day of Debio 1143 in arm A) will be permitted, down to a minimum dose of 5 ml/day (corresponding to 100 mg/day of Debio 1143 in arm A). If further dose reduction is required due to toxicity of at least grade 2, the Debio 1143/matched placebo administration will be permanently discontinued.

Once Debio 1143 or matched placebo dose has been reduced, patient will keep receiving the same dose until further dose reduction is required, or until permanent dose discontinuation due to toxicity occurs, or until the end of study treatment. No re-escalation of Debio 1143/matched placebo dose after dose reduction will be allowed during the study.

During the combination treatment period, if IMRT is interrupted, Debio 1143/matched placebo should be interrupted. If IMRT is restarted, Debio 1143 should be resumed at the next intake day of Debio 1143 that was originally scheduled. If IMRT is permanently discontinued, then Debio 1143/matched placebo administration should be maintained ONLY if the patient has received at least 70% of the total IMRT planned dose. Otherwise Debio 1143/matched placebo treatment should be discontinued, the EOT visit should be performed and the patient should stay in the study for follow-up as per protocol.

The number of treatment days with Debio 1143 intake should not exceed 42 days/period (combination treatment period and monotherapy period).

CT discontinuation will NOT result in Debio 1143/matched placebo discontinuation.

### Chemotherapy

All patients will receive high dose cisplatin (100 mg/m²) at least during the first cycle. Before each administration, creatine clearance should be >59 ml/min/1.73 m²; the patient should not present any neurotoxicity or ototoxicity of grade 2 or above.

High dose cisplatin (commercial formulation) will be administered on Day 2 of each cycle (each cycle is composed of 21 days) for 3 cycles (FIG. 5). The first administration should take place on Day 2 of the first cycle. In cycle 2 and 3, cisplatin can be administered on D2 or D3. Patients who will have received 3 cycles of cisplatin will be considered as having completed CT.

High dose cisplatin should be administered at a dose of 100 mg/m² but the dose should be capped at a maximum of 200 mg if the BSA is 2.00 m² or more. The intravenous (IV) infusion should be administered over at least 90 minutes with pre- and post-infusion hydration+/−mannitol and/or diuretics. The administration should occur at least 30 minutes after Debio 1143/matched placebo and before IMRT.

Cisplatin will require aggressive hydration. Any pre-existing dehydration should be corrected.

Guidance for switching to carboplatin in case a patient is not eligible to continue cisplatin treatment at Cycle 2 and 3 is provided in Table 4.

TABLE 4

| Toxicity type | Parameter | Action | Additional guidance |
|---|---|---|---|
| Hematological | Febrile neutropenia | 1. Interrupt treatment until | Granulocyte colony- |
| toxicity | | resolution of at least Grade 2. | stimulating factor (G-CSF) is |
| | | 2a. For patient receiving cisplatin | permitted after the first course |
| | | AND with | of treatment for patients who |
| | | GFR >60 ml/min/1.73 m², resume | should experience Grade 4 |
| | | cisplatin at 70 mg/m². Maximum | neutropenia (≤1.0 × 10⁹/L) or |
| | | dose is 140 mg. | who developed neutropenic |
| | | 2b. For patient receiving cisplatin | fever between cycles of |
| | | AND with | chemotherapy; G-CSF should |
| | | GFR <60 ml/min/1.73 m², switch to | be given according to |
| | | carboplatin at AUC = 4.5 or 5. | institution guidelines. |
| | | 2c. For patient receiving | The use of recombinant |
| | | carboplatin, resume at AUC = 4-3.5. | human erythropoietin (rHu |
| | | | EPO) is not allowed. |
| | | | In case of grade 3 toxicity |
| | | | further to dose reduction, a |
| | | | second dose reduction down to |
| | | | 50 mg/m² and capped at 100 |
| | | | mg/dose is allowed. |
| | | | For patients receiving |
| | | | carboplatin an equivalent dose |
| | | | reduction can be applied. |
| | Grade 4 | 1. Interrupt treatment until | In case of grade 3 toxicity |
| | thrombocytopenia | resolution of at least Grade 2. | further to dose reduction, a |
| | | 2a. For patient receiving cisplatin | second dose reduction down to |
| | | AND with | 50 mg/m² and capped at 100 |
| | | GFR >60 ml/min/1.73 m², resume | mg/dose is allowed. |
| | | cisplatin at 70 mg/m². Maximum | For patients receiving |
| | | dose is 140 mg. | carboplatin an equivalent dose |
| | | 2b. For patient receiving cisplatin | reduction can be applied. |
| | | AND with | |
| | | GFR <60 ml/min/1.73 m², switch to | |
| | | carboplatin at AUC = 4.5 or 5. | |
| | | 2c. For patient receiving | |
| | | carboplatin, resume at AUC = 4-3.5. | |
| | Grade 4 anemia | 1. Interrupt treatment until | |
| | | resolution of at least Grade 2. | |
| | | 2a. For patient receiving cisplatin | |
| | | AND with | |
| | | GFR >60 ml/min/1.73 m², resume | |
| | | cisplatin at 70 mg/m². Maximum | |
| | | dose is 140 mg. | |
| | | 2b. For patient receiving cisplatin | |
| | | AND with | |
| | | GFR <60 ml/min/1.73 m², switch to | |
| | | carboplatin at AUC = 4.5 or 5. | |
| | | 2c. For patient receiving | |
| | | carboplatin, resume at AUC = 4-3.5. | |
| Renal | Creatinine increase | For patient receiving cisplatin | In case of grade 3 toxicity |
| impairment | | AND with | further to dose reduction, a |
| | | GFR >60 ml/min/1.73 m², resume | second dose reduction down to |
| | | cisplatin at 70 mg/m². Maximum | 50 mg/m² and capped at 100 |
| | | dose is 140 mg. | mg/dose is allowed. |
| | | For patient receiving cisplatin | |
| | | AND with | |
| | | GFR <60 ml/min/1.73 m², switch to | |
| | | carboplatin at AUC = 4.5 or 5. | |
| | | For patient receiving carboplatin, | |
| | | resume at AUC = 4-3.5. | |
| | Glomerular filtration | Switch to carboplatin at AUC = 4.5 | In C2 and C3, carboplatin |
| | rate (GFR) <59 mL/ | or 5. Before each administration, | should be administered on D 2 |
| | mL/min/1.73 m² body | creatinine clearance should be | (D 23 and D 44 of study, |
| | surface area (BSA) | 30-59 ml/min/1.73 m²; the patient | respectively) or D 3 (D 24 and |
| | | should not present any | D 45 of study, respectively), as |
| | | neurotoxicity or ototoxicity of | an IV infusion over at least 30 |
| | | grade 2 or above. | minutes, as per institutional |
| | | | guidelines. |
| | | | Carboplatin should be |
| | | | administered at least 30 |
| | | | minutes after Debio 1143/ |
| | | | matched placebo and should |
| | | | be given before IMRT. |
| Neurotoxicity | Peripheral neuropathy | Reduce dose to 70 mg/m². | Dose adjustments for |
| | Grade 2 | | neurotoxicity will be based on |
| | Peripheral neuropathy | Discontinue cisplatin. | signs and symptoms on the |
| | Grade ≥3 | | day of treatment. |
| | Tinnitus | If it resolves between cycles, | |
| | | continue treatment at 100% dose. | |
| | | If it persists, stop the treatment. | |
| | Hearing impairment | Switch to carboplatin at AUC = 4.5 | In cycle 2 and 3, carboplatin |
| | | or 5 | should be administered on D 2 |
| | | | or D 3, as an IV infusion over |
| | | | at least 30 minutes, as per |
| | | | institutional guidelines, with |
| | | | concomitant IMRT. |
| | | | Carboplatin should be |
| | | | administered at least 30 |
| | | | minutes after Debio 1143/ |
| | | | matched placebo and should |
| | | | be given before radiotherapy. |
| Cardiac toxicity | Symptomatic | Treatment should be permanently | |
| | arrhythmia, | discontinued but the patient | |
| | atrioventricular block | should remain in the study for | |
| | (except first degree) or | follow-up as per protocol. | |
| | documented ischemic | | |
| | event | | |
| Liver | | No dose adjustments are | |
| impairment | | necessary. | |
| Nausea or | | 1. Interrupt treatment until | In C2 and C3, carboplatin |
| vomiting with | | resolution of at least Grade 2. | should be administered on D 2 |
| optimal | | 2a. For patient receiving cisplatin | (D 23 and D 44 of study, |
| prophylaxis | | AND with | respectively) or D 3 (D 24 and |
| | | GFR >60 ml/min/1.73 m², resume | D 45 of study, respectively), as |
| | | cisplatin at 70 mg/m². Maximum | an IV infusion over at least 30 |
| | | dose is 140 mg. | minutes, as per institutional |
| | | 2b. For patient receiving cisplatin | guidelines. |
| | | AND with | Carboplatin should be |
| | | GFR <60 ml/min/1.73 m², switch to | administered at least 30 |
| | | carboplatin at AUC = 4.5 or 5. | minutes after Debio 1143/ |
| | | 2c. For patient receiving | matched placebo and should |
| | | carboplatin, resume at AUC = 4-3.5. | be given before IMRT. |
| | | Before each administration, | |
| | | creatinine clearance should be | |
| | | 30-59 ml/min/1.73 m²; the patient | |
| | | should not present any | |
| | | neurotoxicity or ototoxicity of | |
| | | grade 2 or above. | |

Carboplatin dosing guidance is as follows. No pre- or post-treatment hydration or forced diuresis is required for carboplatin administration.

$$Calvert Equation$$

$$Carboplatin Dose ( mg ) = Target area under the curve ( AUC mg \cdot min / mL ) \times {( {GFR}^{*} + 25 )}^{*} GFR estimated by calculated creatinine clearance \\ \\ using Cockcroft - Gault Equation ( see below ) .$$

$$Cockcroft - Gault Equation$$

$$CrCl ( male ; mL / min ) = ( 140 - age ) \times ( weight in kg )$$

$$72 \times serum creatinine ( mg / dL )$$

$$CrCl ( female ; mL / min ) = 0.85 \times CrCl ( male )$$

### Maximum Carboplatin Dose Calculation

The FDA has recommended that physicians consider capping the dose of carboplatin for desired exposure (AUC) to avoid potential toxicity due to overdosing. The maximum dose is based on a GFR estimate that is capped at 125 mL/min for patients with normal renal function.

Based on the Calvert formula described in the carboplatin label, the maximum doses can be calculated as:

$$Maximum Carboplatin Dose ( mg ) = Target AUC ( mg \cdot min / mL ) \times ( 125 mL / min + 25 )$$

For a target AUC=6, the maximum dose is 6×150=900 mg
For a target AUC=5, the maximum dose is 5×150=750 mg
For a target AUC=4, the maximum dose is 4×150=600 mg

### Additional Considerations

Overweight or obese patients (BMI ?25 kg/m2): Consider using an adjusted body weight.

$$Adjusted body weight ( kg ) = ideal body weight ( IBW ) + 0.4 \times ( total body weight [ TBW ] - IBW )$$

Patients with abnormally low serum creatinine (Cr), including elderly or cachectic patients: Consider using a minimum Cr of 0.7 mg/dL to avoid overestimation of CrCl.

Measured CrCl: Consider using ethylene diamine tetraacetic acid (EDTA) or a 24-hour urine to measure CrCl (not a serum creatinine-based mathematical equation) when dosing at an AUC greater than 6 or when using an un-capped CrCl.

Pre and post 5-HT3 receptor antagonists, such as antagonisetron or palanosetron are allowed if foreseen in institutional guidelines, but ondansetron should be avoided.

### Dose Modification, Interruption and Discontinuation of Chemotherapy

Expected treatment-related toxic effects are mainly hematological (neutropenia, thrombocytopenia, anemia), neurologic (peripheral neuropathy) and renal (renal dysfunction, renal failure, creatinine elevations).

CT dose adjustments at the start of a cycle should be based on the nadir of hematologic counts or maximum non-hematologic toxicity from the previous treatment cycle. Treatment may be delayed to allow sufficient time for recovery.

A repeated course of cisplatin should not be given until the:
Creatinine clearance ≥50 mL/min
BUN <25 mg/100 mL
Platelets >100×109/L
Neutrophils >1.0×109/L

### Radiotherapy

From Cycle 1 to 3, standard fractionation IMRT (only IMRT is allowed) is to be started on D1 of each cycle after administration of Debio 1143 and CT. Radiotherapy should be delivered over 7 weeks:
to the gross-tumor volume (GTV): 5 fractions per week (5/7 days), 1 fraction per day, 2.0 Gy/fraction, up to a total of 70 Gy is to be delivered
as elective irradiation to locoregional areas: 5 fractions per week (5/7 days), 1 fraction per day, 2.0 Gy/fraction up to 50 Gy.

In case IMRT is put on hold for administrative reasons, the total dose planned can be delivered up to week 9.

Patients need to meet the following criteria to start IMRT on D1C2 and D1C3: ?1000/mm3 (per microliter) ANC (absolute neutrophil count), ≥75000/mm3 (per microliter) platelets, Hb ≥8.0 g/dL, albumin ≥1.8 g/dL, ALT/AST ≤×5 ULN, Total bilirubin ≤×2 ULN.

Planned radiation therapy interruptions are not allowed.

Interruption of IMRT to allow resolution/improvement of radio-chemo toxicities should not exceed 21 treatment days in total.

No more than 2 missing doses should be permitted in one week. If treatment interruptions cannot be avoided, the reasons should be documented.

Interrupted fractions should be replaced as follows:

If two doses are missed in one week, add a second fraction on a day of the same week, provided that a minimum of 6-hour interval between the two fractions is allowed.

### OR

Administer the interrupted fractions at the end of treatment

In any case, no more than 6 fractions will be given in any particular week.

If RT is interrupted for more than 21 treatment days, the EOT visit should be performed. Patient will permanently discontinue Debio 1143 but will remain in the study for follow up, as per protocol.

The study consists of 3 periods:
1. Screening: D-42 to D-1 (6 weeks+1-week window prior to D-42).
2. Treatment: D1 through D140 (9 weeks+9 weeks+1 week)
   a) Combination therapy: 3×3-week cycles of Debio 1143/placebo+concomitant CRT.
   b) Monotherapy: 3×3-week cycles of Debio 1143/placebo monotherapy.
3. Follow-up: Subjects will be followed-up until death, until the last on-study subject reaches his/her 60-months post-randomization visit or discontinues from study, whichever occurs first.

A schedule of the administrations is provided as FIG. 5. In the protocol depicted in FIG. 5, a human patient is administered Compound A on Days 1-14 and no compound A on Days 15-21 of a 21-day cycle. The human patient is also concurrently administered a platinum-based chemotherapeutic drug (e.g., cisplatin or carboplatin) on only Day 2, as well as radiotherapy on Days 1-5, 8-12, and 15-19 of the 21 day cycle. The 21-day cycle is repeated two additional times (for radiotherapy to be administered for 9 weeks). If radiotherapy is to be provided for only seven or eight weeks, then the last two or one week, respectively, will be omitted during the third cycle Then, three 21-day cycles will follow in which the human patient is administered Compound A for Days 1-14 and no Compound A for Days 15-21 in each 21-day cycle.

In a variant 2 of this Example 6, the study is conducted as described above, and in addition, dose modifications of Debio 1143/matched placebo and/or platinum-based chemotherapy after an Adverse Event (AE) related to study treatment(s) are handled as per Table 5 below during combination treatment period (cycles 1 to 3) and as per Table 6 below during monotherapy (cycles 4 to 6). In this variant 2, Table 4 does not apply regarding cisplatin dose modifications. In this variant 2, the general rules for Debio 1143/placebo administration as described above apply only to the extent that they are in alignment with Tables 5 and 6.

TABLE 5
Debio 1143/matched placebo and platinum-based chemotherapy dose modifications after an
AE related to study treatment(s), during combination treatment period (cycles 1 to 3)

| | Grade (as per | | |
|---|---|---|---|
| | Common Terminology | | |
| | Criteria for Adverse | | |
| | Events [CTCAE] | Action: Treatment modification | |
| Adverse Event | V5.0) or cut-off | Debio 1143 or matched placebo | Cisplatin/Carboplatin |

Blood and lymphatic system disorders

| | | | |
|---|---|---|---|
| Febrile | G3 or G4 | G4: | 1^{st} occurrence |
| neutropenia | | Omit dose of Debio 1143/matched | Hold further chemotherapy until resolution |
| | | placebo until resolution to <G1 | to <G1 fever and ≤G2 neutropenia. |
| | | fever and ≤G 2 neutropenia | If recovered to <G1 fever and ≤G 2 |
| | | (mandatory). | neutropenia, resume treatment at the next |
| | | Resume treatment at the same | lower dose level (−25% from prior dose) |
| | | dose level. | at the next scheduled chemotherapy |
| | | G3: | administration. |
| | | Maintain Debio 1143/1143 matched | For subjects on carboplatin or switching to |
| | | placebo at same dose level at the | carboplatin: resume treatment at AUC = 4. |
| | | Investigator's/Medical Doctor's | Recurrence |
| | | discretion. | Consider permanent discontinuation of |
| | | Otherwise, omit dose of Debio | cisplatin/carboplatin at Investigator's/ |
| | | 1143/matched placebo until | Medical Doctor's discretion. |
| | | resolution to <G1 fever and ≤G2 | Otherwise, if resolved to <G1 fever and ≤G2 |
| | | neutropenia. | neutropenia, resume cisplatin treatment at |
| | | Resume treatment at the same | the next lower dose level (−25% from prior |
| | | dose level. | dose) or consider switching to carboplatin |
| | | | treatment at AUC = 4 (if not already done) |
| | | | at the next scheduled chemotherapy |
| | | | administration. |
| Neutrophil | G4 | Omit dose of Debio 1143/matched | 1^{st} occurrence |
| count decrease | | placebo dose until resolution | Hold further chemotherapy until resolution |
| | | to ≤G2 (mandatory). | to ≤G2. |
| | | Resume treatment at the same | If resolved to ≤G2, resume treatment at the |
| | | dose level. | next lower dose level (−25% from prior dose) |
| | | | at the next scheduled chemotherapy |
| | | | administration. |
| | | | For subjects on carboplatin or switching to |
| | | | carboplatin: resume treatment at AUC = 4. |
| | | | Recurrence |
| | | | Consider permanent discontinuation of |
| | | | cisplatin/carboplatin at Investigator's/ |
| | | | Medical Doctor's discretion. |
| | | | Otherwise: Hold further chemotherapy until |
| | | | resolution to ≤G2, If resolved to ≤G2, resume |
| | | | cisplatin treatment at the next lower dose |
| | | | level (−25% from prior dose) OR consider to |
| | | | switching to carboplatin treatment at AUC = 4 |
| | | | (if not already done) at the next scheduled |
| | | | chemotherapy administration. |
| | | | Note: Use of granulocyte colony-stimulating |
| | | | factor (G-CSF) prophylaxis is recommended if |
| | | | chemotherapy is continued. |
| | G3 | Maintain Debio 1143/matched | Hold further chemotherapy until resolution |
| | | placebo at same dose level at the | to ≤G2. |
| | | Investigator's/Medical Doctor's | If resolved to ≤G2, resume treatment at the |
| | | discretion. Monitor closely. | same dose at the next scheduled chemotherapy |
| | | Otherwise, hold treatment until | administration. |
| | | resolution to ≤G2. Resume | Note: Use of granulocyte colony-stimulating |
| | | treatment at the same dose level. | factor (G-CSF) prophylaxis is recommended if |
| | | | chemotherapy is continued. |
| Thrombocytopenia | G4 | Omit dose of Debio 1143/matched | 1^{st} occurrence |
| | | placebo dose until resolution | Hold further cisplatin administration until |
| | | to ≤G2 (mandatory). | resolution to ≤G2. |
| | | Resume treatment at the same | If resolved to ≤G2, resume treatment at the |
| | | dose level. | next lower dose level (−25% from prior dose) |
| | | | at the next scheduled chemotherapy |
| | | | administration. |
| | | | For subjects on carboplatin or switching |
| | | | to carboplatin: hold administration until |
| | | | platelet count ≥100.000 cells/mm³ and resume |
| | | | treatment at AUC = 4. |
| | | | Recurrence |
| | | | Hold further cisplatin administration until |
| | | | resolution to ≤G2. |
| | | | If resolved to ≤G2 resume treatment at the |
| | | | next lower dose level (−25% from prior dose) |
| | | | at the next scheduled chemotherapy |
| | | | administration. |
| | | | For subjects on carboplatin or switching |
| | | | to carboplatin: hold administration until |
| | | | platelet count ≥100.000 cells/mm³ and resume |
| | | | treatment at AUC = 4. |
| | | | Consider permanent discontinuation of |
| | | | cisplatin/carboplatin treatment at |
| | | | Investigator's/Medical Doctor's discretion. |
| | | | Note: Consider Platelets transfusion as |
| | | | clinically indicated. |
| | G3 | Maintain Debio 1143/matched | Hold further cisplatin administration until |
| | | placebo at same dose level at | resolution to ≤G2. |
| | | the Investigator's/Medical | If resolved to ≤G2, resume treatment at the |
| | | Doctor's discretion. Monitor | same dose at the next scheduled chemotherapy |
| | | closely. | administration as per Investigator's/Medical |
| | | Otherwise, hold treatment until | Doctor's discretion. Otherwise, resume |
| | | resolution to ≤G2. Resume | treatment at the next lower dose level (−25% |
| | | treatment at the same dose level. | from prior dose) at the next scheduled |
| | | | chemotherapy administration. |
| | | | For subjects on carboplatin or switching |
| | | | to carboplatin: hold administration until |
| | | | platelet count ≥100.000 cells/mm³ and resume |
| | | | treatment at AUC = 4. |
| Anemia | G4 | Omit dose of Debio 1143/matched | 1^{st} occurrence |
| | | placebo until resolution to ≤G2 | Hold further chemotherapy until resolution |
| | | (mandatory). | to ≤G2. |
| | | Resume treatment at the next | If resolved to ≤G2, resume treatment at the |
| | | lower dose level. | next lower dose level (−25% from prior dose) |
| | | | at the next scheduled chemotherapy |
| | | | administration. |
| | | | For subjects on carboplatin or switching to |
| | | | carboplatin: resume treatment at AUC = 4. |
| | | | Recurrence |
| | | | Hold further chemotherapy until resolution |
| | | | to ≤G2. |
| | | | Resume treatment at the next lower dose |
| | | | level (−25% from prior dose) at the next |
| | | | scheduled chemotherapy administration. |
| | | | Consider permanent discontinuation of |
| | | | cisplatin/carboplatin therapy at |
| | | | Investigator's/Medical Doctor's discretion. |
| | | | Note: Consider PRBC transfusion as |
| | | | clinically indicated. |
| | | | The use of recombinant human erythropoietin |
| | | | (rHu EPO) is not allowed. |
| | G3 | Maintain Debio 1143 at same dose | 1^{st} occurrence |
| | | level at the Investigator's/ | Hold further chemotherapy until resolution |
| | | Medical Doctor's discretion. | to ≤G2. |
| | | Otherwise, hold treatment | If resolved to ≤G2, resume treatment at |
| | | until resolution to ≤G2. Resume | the same dose level at the next scheduled |
| | | treatment at the same dose level. | chemotherapy administration. |
| | | | For subjects on carboplatin or switching to |
| | | | carboplatin: resume treatment at AUC = −5. |
| | | | Recurrence |
| | | | Hold further chemotherapy until resolution |
| | | | to ≤G2. |
| | | | If resolved to ≤G2, consider resuming |
| | | | treatment at the next lower dose level |
| | | | (−25% from prior dose) at the next |
| | | | scheduled chemotherapy administration, at |
| | | | Investigator's/Medical Doctor's discretion. |
| | | | For subjects on carboplatin or switching to |
| | | | carboplatin: consider resuming treatment at |
| | | | AUC = 4, at Investigator's/Medical Doctor's |
| | | | discretion. |
| | | | Note: Consider PRBC transfusion as |
| | | | clinically indicated. |
| | | | The use of recombinant human erythropoietin |
| | | | (rHu EPO) is not allowed. |

Investigations

| Amylase and/or | G4 | 1^{st} occurrence | 1^{st} occurrence |
|---|---|---|---|
| lipase increase | | Omit dose Debio 1143/matched | Hold further chemotherapy until resolution |
| | | placebo until resolution to ≤G2. | to ≤G2. |
| | | If resolved in ≤7 days: resume | If resolved to ≤G2, resume treatment at the |
| | | treatment at the next lower | next lower dose level (−25% from prior dose) |
| | | dose level. | at the next scheduled chemotherapy |
| | | If resolved in >7 days: | administration. |
| | | permanently discontinue from | For subjects on carboplatin or switching to |
| | | study treatment (mandatory). | carboplatin: resume treatment at AUC = 4. |
| | | Any recurrence | Recurrence |
| | | Permanent discontinuation of Debio | Permanent discontinuation of |
| | | 1143/matched placebo (mandatory). | cisplatin/carboplatin. |
| | G3 symptomatic | 1^{st} occurrence | Action based on Investigator's/Medical |
| | amylase and/or | Omit dose of Debio 1143/matched | Doctor's judgment. |
| | lipase* | placebo until resolution to ≤G2. | |
| | | If resolved in ≤14 days: resume | |
| | | treatment at the next lower dose | |
| | | level. | |
| | | If resolved in >14 days, | |
| | | permanently, discontinue from | |
| | | Debio 1143/matched placebo. | |
| | | Any recurrence | |
| | | Permanent discontinuation of Debio | |
| | | 1143/matched placebo (mandatory). | |
| | G3 asymptomatic | 1^{st} occurrence | 1^{st} occurrence |
| | amylase/lipase* | Maintain dose of Debio | Action based on Investigator's/Medical |
| | | 1143/matched placebo at | Doctor's judgment. |
| | | Investigator's/Medical Doctor's | Any recurrence |
| | | discretion and monitor closely. | Permanent discontinuation of |
| | | Otherwise, omit dose of Debio | cisplatin/carboplatin. |
| | | 1143/matched placebo until | |
| | | resolution to ≤G2. | |
| | | If resolved in ≤7 days: resume | |
| | | treatment at the same dose level. | |
| | | If resolved in >7 days: resume | |
| | | treatment at the next lower | |
| | | dose level. | |
| | | Any recurrence | |
| | | Permanent discontinuation of | |
| | | Debio 1143/matched placebo. | |
| | *Note: A CT-scan or other imaging study to assess the pancreas, liver, | | |
| | and biliary tract must be performed within 1 week of the first occurrence | | |
| | of any CTCAE ≥G3 amylase and/or lipase, or as clinically indicated. | | |
| Liver toxicity: | G4 | Permanent discontinuation of Debio | Permanent discontinuation of |
| AST and/or ALT | | 1143/matched placebo (mandatory). | cisplatin/carboplatin. |
| | G3 | 1^{st} occurrence | Action based on Investigator's/Medical |
| | | Omit dose of Debio 1143/matched | Doctor's judgment. |
| | | placebo until resolution to ≤G1. | |
| | | If resolved in ≤14 days, resume | |
| | | treatment at the same dose level. | |
| | | If resolved in >14 days, resume | |
| | | treatment at the next lower dose | |
| | | level. | |
| | | Any recurrence | |
| | | Omit dose of Debio 1143/matched | |
| | | placebo until resolution to ≤G1. | |
| | | Resume treatment at the next | |
| | | lower dose level. | |
| Hy's Law criteria | AST increased ≥G2, | Omit dose of Debio 1143/matched | Action based on Investigator's/Medical |
| for DILI | and/or | placebo. | Doctor's judgment. |
| | ALT increased ≥G2, | Assess any potential cause of | |
| | and | liver toxicity: | |
| | Total bilirubin | If any potential cause other | |
| | increased >2 × ULN | than DILI is identified and | |
| | and | removed: Resume treatment at | |
| | ALP increased ≤2 × | the same dose level only after | |
| | ULN | resolution to ≤G1 for ALT/AST | |
| | | AND Total bilirubin ≤2 × ULN. | |
| | | Monitor closely. | |
| | | If re-occurrence after treatment | |
| | | re-introduction, then permanently | |
| | | discontinue Debio 1143/matched | |
| | | placebo (mandatory). | |
| | | If no other cause has been | |
| | | identified, permanently | |
| | | discontinue Debio 1143/matched | |
| | | placebo (mandatory). | |
| Torsade de pointes; | G4 | Permanent discontinuation of Debio | Permanent discontinuation of |
| polymorphic | | 1143/matched placebo (mandatory). | cisplatin/carboplatin. |
| ventricular | | | |
| tachycardia; | | | |
| signs/symptoms of | | | |
| serious arrhythmia | | | |
| QTcF-prolongation: | G3 | If the QTcF prolongation is | 1^{st} occurrence |
| Note: A QT interval | QTcF >500 ms or | confirmed by either criterion | Action based on Investigator's/Medical |
| is confirmed as | QT interval | (i.e., QTcF interval >500 ms | Doctor's judgment. |
| prolonged if ≥2 out | prolonged >60 ms | or >60 ms increase from baseline): | 2^{nd} occurrence |
| of 3 ECG traces show | from baseline | Omit dose of Debio 1143/matched | Consider permanent discontinuation of |
| a QTcF interval | | placebo until resolution to within | cisplatin/carboplatin at Investigator's/ |
| prolongation. | | 30 ms from baseline or QTcF <480 ms. | Medical Doctor's discretion. |
| | | Consult the medical monitor prior | |
| | | to administering further treatment | |
| | | doses. | |
| | | If the QTcF interval does not | |
| | | return to within 30 ms of baseline | |
| | | or <480 ms within 14 days, | |
| | | permanently discontinue Debio | |
| | | 1143/matched placebo. | |
| | | If the prolongation is confirmed | |
| | | by both criteria (i.e., QTcF | |
| | | interval >500 ms and >60 ms | |
| | | increase from baseline): | |
| | | Permanently discontinue Debio | |
| | | 1143/matched placebo. | |

Nervous system disorders

| | | | |
|---|---|---|---|
| Neurotoxicity: | G3 or G4 | Maintain dose of Debio 1143/ | 1^{st} occurrence |
| peripheral | | matched placebo at the | Permanent discontinuation of cisplatin and |
| neuropathy | | Investigator's/Medical Doctor's | switch to carboplatin AUC = 5 at the next |
| | | discretion and monitor closely. | chemotherapy administration. |
| | | | For subjects already on carboplatin: resume |
| | | | treatment at AUC = 4. |
| | | | Recurrence |
| | | | Consider permanent discontinuation of |
| | | | carboplatin at Investigator's/Medical |
| | | | Doctor's discretion. |
| | G2 | Maintain dose Debio 1143/matched | 1^{st} occurrence |
| | | placebo at the Investigator's/ | Hold further chemotherapy until resolution |
| | | Medical Doctor's discretion. | to ≤G1. |
| | | | If resolved to ≤G1, resume treatment at the |
| | | | next lower dose level (−25% from prior dose) |
| | | | at the next scheduled chemotherapy |
| | | | administration. |
| | | | OR switch to carboplatin AUC = 5. |
| | | | For subjects already on carboplatin: resume |
| | | | treatment at AUC = 4. |
| | | | Recurrence |
| | | | Consider permanent discontinuation of |
| | | | cisplatin/carboplatin at Investigator's/ |
| | | | Medical Doctor's discretion. |

Renal impairment

| | | | |
|---|---|---|---|
| eGFR | 30-59 ml/min/1.73 m2 | Maintain dose Debio 1143/matched | 1^{st} occurrence |
| (using the CKD-EPI | | placebo. | Switch to carboplatin AUC = 5 at the next |
| creatinine formula) | | | scheduled chemotherapy administration. |
| | | | Or |
| | | | For subject continuing on cisplatin: lower |
| | | | the dose to 50 mg/m² at the next scheduled |
| | | | chemotherapy administration as per |
| | | | Investigator's/Medical Doctor's discretion. |
| | | | Recurrence |
| | | | Consider permanent discontinuation of |
| | | | cisplatin/carboplatin or consider switching |
| | | | to carboplatin treatment at AUC = 5 (if not |
| | | | already done) at the next scheduled |
| | | | chemotherapy administration. |
| | <30 ml/min/1.73 m2 | Omit of dose of Debio 1143/matched | Permanent discontinuation of cisplatin. |
| | | placebo dose until eGFR to ≥30 | Consider switching to carboplatin AUC = 5 |
| | | ml/min/1.73 m². | at the next chemotherapy administration. |
| | | Resume treatment at the same dose | |
| | | level. | |

Gastrointestinal disorders

| | | | |
|---|---|---|---|
| Nausea and/or | G4 | Permanent discontinuation of Debio | Permanent discontinuation of |
| Vomiting | | 1143/matched placebo (mandatory). | cisplatin/carboplatin (mandatory). |
| | G3 persisting for | Omit of dose of Debio 1143/matched | 1^{st} occurrence |
| | 5 days despite | placebo until resolution to ≤G2. | Hold chemotherapy until resolution to ≤G2. |
| | adequate | Resume treatment at the same dose | If resolved to ≤G2, resume treatment at the |
| | supportive therapy | level. | same dose level at the next scheduled |
| | | | chemotherapy administration OR switch to |
| | | | carboplatin AUC = 4. |
| | | | For subjects already on carboplatin: resume |
| | | | treatment at AUC = 4. |
| | | | Recurrence |
| | | | For subject receiving cisplatin: switch |
| | | | to carboplatin AUC = 4. |
| | | | OR consider permanent discontinuation of |
| | | | cisplatin/carboplatin at Investigator's/ |
| | | | Medical Doctor's discretion. |
| Diarrhea | G4 | Permanent discontinuation of Debio | Permanent discontinuation of |
| | | 1143/matched placebo (mandatory). | cisplatin/carboplatin. |
| | G3 | Omit of dose of Debio 1143/matched | 1^{st} occurrence |
| | persisting for 5 | placebo until resolution to ≤G2. | Hold chemotherapy until resolution to ≤G2. |
| | days despite | Resume treatment at the same dose | Resume treatment at the same dose level at the |
| | adequate | level. | next scheduled chemotherapy administration |
| | supportive therapy | | OR switch to carboplatin AUC = 4. |
| | | | For subjects on carboplatin or switching to |
| | | | carboplatin: resume treatment at AUC = 4. |
| | | | Recurrence |
| | | | For subject receiving cisplatin: switch to |
| | | | carboplatin AUC = 4. |
| | | | OR consider permanent discontinuation of |
| | | | cisplatin/carboplatin at Investigator's/ |
| | | | Medical Doctor's discretion. |
| Mucositis/Dysphagia | G4 | Omit dose of Debio 1143/matched | Hold chemotherapy until resolution to ≤G3. |
| | | placebo dose until resolution | If resolved to ≤G3, resume treatment at the |
| | | to ≤G3. | next lower dose level (−25% from prior dose) |
| | | If resolved to ≤G3, resume | at the next scheduled chemotherapy |
| | | treatment at the same dose level | administration at Investigator's/Medical |
| | | at the Investigator's/Medical | Doctor's discretion. |
| | | Doctor's discretion. Otherwise, | |
| | | resume treatment at the next | |
| | | lower dose level. | |

Note: A percutaneous endoscopic gastrostomy or nasogastric
tube should be placed according to potential weight loss
and swallowing impairment (if not already in place).

Ear and labyrinth disorders

| | | | |
|---|---|---|---|
| Tinnitus | ≥G2 | Maintain dose of Debio | Hold chemotherapy until resolution to ≤G1. |
| | | 1143/matched placebo. | For subject receiving cisplatin: |
| | | | If recovered to ≤G1, lower the dose to 50 |
| | | | mg/m² at the next scheduled chemotherapy |
| | | | administration as per Investigator's/Medical |
| | | | Doctor's discretion or switch to carboplatin: |
| | | | resume treatment at AUC = 5. |
| | | | If tinnitus persists, discontinue cisplatin |
| | | | treatment (mandatory) and switch to |
| | | | carboplatin: resume treatment at AUC = 4. |
| Hearing impaired | ≥G2 | Maintain dose of Debio | 1^{st} occurrence |
| | | 1143/matched placebo. | Permanent discontinuation of cisplatin and |
| | | | switch to carboplatin AUC = 5 at the next |
| | | | chemotherapy administration. |
| | | | For subjects on carboplatin or switching to |
| | | | carboplatin: resume treatment at AUC = 4. |
| | | | Recurrence |
| | | | Consider permanent discontinuation of |
| | | | carboplatin at Investigator's/Medical |
| | | | Doctor's discretion. |
| | | | For subjects on carboplatin: resume |
| | | | treatment at AUC = 4. |

All other adverse events (AEs) (suspected to be related
to study treatment including Debio 1143/matched placebo)

| | | | |
|---|---|---|---|
| Other AEs | G4 | Permanent discontinuation of Debio | Action based on Investigator's/Medical |
| | | 1143/matched placebo (mandatory). | Doctor's judgment. |
| | | A subject with a G4 AE may only | |
| | | resume treatment at the lower dose | |
| | | level if in the opinion of the | |
| | | Investigator/Medical Doctor and | |
| | | after discussion with the Sponsor | |
| | | and documentation, the event is | |
| | | not life-threatening, and the | |
| | | subject can be managed and | |
| | | monitored for recurrence of AE. | |
| | G3 | Omit dose of Debio 1143/matched | Action based on Investigator's/Medical |
| | | placebo dose until resolution | Doctor's judgment. |
| | | to ≤G2. | |
| | | If resolved in ≤7 days, resume | |
| | | treatment at the same dose level | |
| | | at the Investigator's/Medical | |
| | | Doctor's discretion. | |
| | | If resolved in >7 days, resume | |
| | | treatment at the next lower dose | |
| | | level. | |

Abbreviations: AE: adverse event; ALP: alkaline phosphatase; ALT: alanine aminotransferase; AST: aspartate aminotransferase; AUC: area under the curve; C: cycle; DILI: drug-induced liver injury; eGFR: estimated glomerular filtration rate; G: grade; EPO: erythropoietin; PRBC: packed red blood cells; QTcF: corrected QT interval by Fredericia; ULN: upper limit of normal.

TABLE 6
Debio 1143/matched placebo dose modifications after an AE related to study
treatment(s), during monotherapy treatment period (Cycles 4 to 6)

| | | |
|---|---|---|
| | Grade (as per CTCAE | |
| Adverse Event | V5.0) or cut-off | Action: Treatment modification |

Debio 1143 or matched placebo
Investigations

| Amylase and/or | G4 amylase and/or lipase | 1^{st} occurrence |
|---|---|---|
| lipase | | Omit dose of Debio 1143/matched placebo |
| | | until resolution to ≤G2. |
| | | If resolved in ≤7 days: resume treatment |
| | | at the next lower dose level. |
| | | If resolved in >7 days: permanently discontinue |
| | | Debio 1143/matched placebo (mandatory). |
| | | Any recurrence |
| | | Permanent discontinuation of Debio 1143/matched |
| | | placebo (mandatory). |
| | G3 symptomatic amylase/lipase | 1^{st} occurrence |
| | | Omit dose of Debio 1143/matched placebo |
| | | until resolution to ≤G2. |
| | | If resolved in ≤14 days: resume treatment |
| | | at the next lower dose level. |
| | | If resolved in >14 days, permanently, |
| | | discontinue Debio 1143/matched placebo. |
| | | Any recurrence |
| | | Permanent discontinuation of Debio 1143/matched |
| | | placebo (mandatory). |
| | G3 asymptomatic amylase/lipase | 1^{st} occurrence |
| | | Maintain dose of Debio 1143/matched placebo |
| | | at the Investigator's/Medical Doctor's |
| | | discretion and monitor closely. |
| | | Otherwise, omit dose of Debio 1143/matched |
| | | placebo until resolution to ≤G2. |
| | | If resolved in ≤7 days: resume treatment |
| | | at the same dose level. |
| | | If resolved in >7 days: resume treatment |
| | | at the next lower dose level. |
| | | Any recurrence |
| | | Permanent discontinuation of Debio 1143/matched |
| | | placebo. |
| | Note: A CT-scan or other imaging study to assess the pancreas, liver, | |
| | and gallbladder must be performed within 1 week of the first occurrence | |
| | of any CTCAE ≥G3 amylase and/or lipase, or as clinically indicated. | |
| AST and/or ALT | G4 | Permanent discontinuation of Debio 1143/matched |
| | | placebo (mandatory). |
| | G3 | 1^{st} occurrence |
| | | Omit dose of Debio 1143/matched placebo |
| | | until resolution to ≤G1. |
| | | If resolved in ≤14 days, resume treatment |
| | | at the same dose level. |
| | | If resolved in >14 days, resume treatment |
| | | at the next lower dose level. |
| | | Any recurrence |
| | | Omit dose of Debio 1143/matched placebo |
| | | until resolution ≤G1. |
| | | Resume treatment at the next lower dose level. |
| Hy's Law criteria | AST increased ≥G2, | Omit dose of Debio 1143/matched placebo. |
| for DILI | and/or | Assess any potential cause of liver toxicity: |
| | ALT increased ≥G2, | If any potential cause other than DILI is |
| | and | identified and removed: Resume treatment |
| | Total bilirubin | at the same dose level only after resolution |
| | increased >2 × ULN | to ≤G1 for ALT/AST AND Total bilirubin ≤2 × |
| | and | ULN. Monitor closely. |
| | ALP increased ≤2 × ULN | If re-occurrence after treatment |
| | | re-introduction then permanently discontinue |
| | | Debio 1143/matched placebo (mandatory). |
| | | If no other cause has been identified, |
| | | permanently Debio 1143/matched placebo |
| | | (mandatory). |
| Torsade de pointes; | G4 | Discontinue Debio 1143/matched placebo |
| polymorphic | | permanently |
| ventricular | | |
| tachycardia; | | |
| signs/symptoms of | | |
| serious arrhythmia | | |
| QTcF prolongation | G3 | 1^{st} occurrence |
| Note: A QT interval | QTcF >500 ms or QT interval | Omit dose of Debio 1143/matched placebo |
| is confirmed as | prolonged >60 ms | until QTcF <480 ms. |
| prolonged if ≥2 out | | Stop any concomitant medication known to |
| of 3 ECG traces | | potentially cause QTc prolongation. |
| show a QTcF interval | | If no concomitant medication known to |
| prolongation. | | potentially cause QTc prolongation: consider |
| | | resuming treatment at the next lower dose level. |
| | | 2^{nd} occurrence |
| | | Permanent discontinuation of Debio 1143/matched |
| | | placebo (mandatory). |

[symbol]

Gastrointestinal disorders

| | | |
|---|---|---|
| Nausea/Vomiting, | G3 refractory to supportive | Omit dose of Debio 1143/matched placebo until |
| despite adequate | care | resolution ≤G2. |
| standard prophylaxis | | Resume treatment at the next lower dose level. |

All other AEs (suspected to be related to Debio 1143/matched placebo)

| | | |
|---|---|---|
| Other AEs | G4 | Permanent discontinuation of Debio 1143/matched |
| | | placebo (mandatory). |
| | | A subject with a G4 AE may only resume treatment |
| | | at the lower dose level if in the opinion of the |
| | | Investigator and after discussion with the |
| | | Sponsor and documentation, the event is not |
| | | life-threatening, and the subject can be managed |
| | | and monitored for recurrence of AE. |
| | G3 | Omit dose of Debio 1143/matched placebo dose |
| | | until resolution to ≤G2. |
| | | If resolved in ≤7 days, resume treatment at the |
| | | same dose level at the Investigator's discretion. |
| | | If resolved in >7 days, resume treatment at the |
| | | next lower dose level. |

Abbreviations: AE: adverse event; ALP: alkaline phosphatase; ALT: alanine aminotransferase; AST: aspartate aminotransferase; AUC: area under the curve; C: cycle; DILI: drug-induced liver injury; G: grade; QTcF: corrected QT interval by Fredericia; ULN: upper limit of normal.

It is to be appreciated that the Detailed Description section, and not the Summary and Abstract sections, is intended to be used to interpret the claims. The Summary and Abstract sections sets forth one or more, but not all, exemplary aspects of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the invention that others can, by applying knowledge within the skill of the art, readily modify and/or adapt for various applications such specific aspects, without undue experimentation, without departing from the general concept of the present invention. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary aspects, but should be defined only in accordance with the following claims and their equivalents.

1. A method for treating a human patient having locally advanced squamous cell carcinoma comprising administering to the human patient, in need thereof, one or more inhibitors of apoptosis protein (“IAP”) antagonists, wherein the method comprises:
(a) a first regimen comprising concomitant administration of the one or more IAP antagonists, a platinum-based chemotherapeutic drug, and radiation; and
(b) a second regimen comprising administration of the one or more IAP antagonists.

2. The method of claim 1, wherein the first regimen comprises at least one 21-day cycle in which the one or more IAP antagonists is administered for 14 consecutive days followed by seven days in which no IAP antagonist is administered.

3. The method of claim 2, wherein the first regimen comprises three consecutive 21-day cycles.

4. The method of claim 3, wherein the platinum-based chemotherapeutic drug of the first regimen is administered on the first day of each 21-day cycle.

5. The method of claim 3, wherein the platinum-based chemotherapeutic drug of the first regimen is administered on the second day of each 21-day cycle.

6. The method of claim 3, wherein radiation is administered on the first five consecutive days of each 21-day cycle.

7. The method of claim 1, wherein the second regimen comprises at least one 21-day cycle in which at least one IAP antagonist is administered for 14 consecutive days followed by seven days in which no IAP antagonist is administered.

8. The method of claim 7, wherein the second regimen comprises two or three consecutive 21-day cycles.

9. (canceled)

10. The method of claim 1, wherein no platinum-based chemotherapeutic drug or radiation is administered in the second regimen.

11-14. (canceled)

15. The method of claim 1, wherein the human patient is administered at least one IAP antagonist that is Compound A, or pharmaceutically acceptable salt thereof, in an amount corresponding to about 100 mg to about 500 mg of Compound A as its free base, daily.

16.-37. (canceled)

38. The method of claim 1, wherein the platinum-based chemotherapeutic drug of the first regimen is one or more selected from the group consisting of cisplatin, carboplatin, oxaliplatin, and nedaplatin.

39.-53. (canceled)

54. The method of claim 1, wherein the locally advanced squamous cell carcinoma is of the head and neck region.

55.-58. (canceled)

59. The method of claim 1, wherein the locally advanced squamous cell carcinoma is Stage III, Stage IVA, or Stage IVB according to the American Joint Committee on Cancer 1998 staging system or wherein the locally advanced squamous cell carcinoma is Stage III, Stage IVA, or Stage IVB according to the American Joint Committee on Cancer 2010 or 2018 staging system.

60.-112. (canceled)

113. A method for treating a human patient having locally advanced squamous cell carcinoma comprising administering to the human patient, in need thereof, Compound A, or pharmaceutically acceptable salt thereof, wherein the method comprises:
(a) a first regimen comprising three 21-day cycles that comprise administering Compound A, or pharmaceutically acceptable salt thereof, on Days 1-14 of each cycle, cisplatin on Day 2 of each cycle, and radiation on Days 1-5, 8-12, and 15-19 of each cycle, wherein cisplatin may be replaced by carboplatin after the first cycle; and
(b) a second regimen comprising three 21-day cycles that comprise administering Compound A, or pharmaceutically acceptable salt thereof, on Days 1-14 of each cycle.

114. The method of claim 113, wherein the human patient is administered Compound A, or pharmaceutically acceptable salt thereof, in an amount corresponding to about 100 mg to about 500 mg of Compound A as its free base, daily.

115.-145. (canceled)

146. The method of claim 113, wherein the locally advanced squamous cell carcinoma is of the head and neck region.

147.-234. (canceled)

235. A method for treating a human patient having locally advanced squamous cell carcinoma, wherein the method comprises administering to the patient Compound A, or pharmaceutically acceptable salt thereof, cisplatin or carboplatin, and IMRT for at least one cycle of 21 days.

236.-251. (canceled)

252. A kit comprising: (a) a first pharmaceutical composition comprising an inhibitor of apoptosis protein (“IAP”) antagonist (e.g. Compound A or a pharmaceutically acceptable salt thereof); and (b) an insert instructing use of the first pharmaceutical composition for treating locally advanced squamous cell carcinoma via the method of claim 1.
